(19) United States
(12) Patent Application Publication
(10) Pub. No.: US 2024/0239829 A1
(43) Pub. Date: Jul. 18, 2024

(54) APPLICATIONS OF ANALOGS OR DERIVATIVES OF SIALIC ACIDS

(71) Applicant: B & H Biotechnologies, LLC, Willowbrook, IL (US)

(72) Inventor: Huiru WANG, Willowbrook, IL (US)

(73) Assignee: B & H Biotechnologies, LLC, Willowbrook, IL (US)

(21) Appl. No.: 18/565,044

(22) PCT Filed: Apr. 15, 2022
(86) PCT No.: PCT/CN2022/087136

§ 371 Date: Nov. 28, 2023

(30) Foreign Application Priority Data
May 31, 2021 (WO) PCT/CN2021/097348

(51) Int. Cl. C07H 13/04; A61K 31/7012; A61K 47/12; A61K 47/26; A61P 19/02; A61P 31/14; A61P 31/16

(52) U.S. Cl. CPC C07H 13/04; A61K 31/7012; A61K 47/12; A61K 47/26; A61P 19/02; A61P 31/14; A61P 31/16

(57) ABSTRACT

Provided are compositions or products comprising analogs or derivatives of N-acetylneuraminic acid with a particular pH range. The compositions or products are effective for treating or preventing highly pathogenic viral infections such as COVID-19 infection, the serious adverse reactions of vaccines, and infection-relating autoimmune diseases including COVID-19 long haulers. More specifically, the present disclosure relates to the methods of preparing the compositions or the products. Products can be implemented as a therapeutic product, a nutritional supplement, a food, a feed, a food additive, a feed additive, a rehydration salt, or a rehydration solution.

### CROSS-REFERENCE TO RELATED APPLICATIONS

This application claims the priority benefit of International Application No. PCT/CN2021/097348, filed May 31, 2021, which is incorporated herein by reference in its entirety.

### FIELD

The present disclosure is related generally to the fields of biological and medical technology, specifically related to compositions and products comprising the analogs or derivatives of sialic acid, and the use of the compositions or the products for preventing and/or treating saccharate related diseases in particular respiratory and gastrointestinal viral infections, adverse reactions of vaccines, and infection-relating autoimmune diseases. More specifically, the present disclosure relates to the methods of preparing the compositions or the products containing the compositions.

### BACKGROUND

Saccharides are found widely distributed in human or animal tissues, especially in glycoproteins and gangliosides. Cell surface glycans function as signaling molecules, recognition molecules and adhesion molecules (Sharon and Lis, 1993; Ofek et al., 2003). Many cell surface proteins are modified by the addition of saccharides, a process termed protein glycosylation. Nearly all types of malignant cells and many types of diseased tissues demonstrate alterations in their glycosylation patterns (Blomme et al., 2009; Danussi et al., 2009; Patsos et al., 2009). In recent years, therapeutic products targeting saccharide targets have attracted wide attention. How specific cell surface proteins are modified by the glycosylation of saccharides, and how patterns of glycosylation change affect a physiological or a pathological process are all problems that are just beginning to be explored (for a review, see Moremen, K. W., et al. (2012) Nat. Rev. Mol. Cell Biol. 13(7):448-62; Roland Schauer & Johannis P. Kamerling. Exploration of the sialic acid world. Elsevier, 2018, 12.1).

An infectious disease is a clinically evident disease of humans or animals. A highly pathogenic virus can cause a worldwide pandemic which threats people's health and economy globally. The clinic condition of some patients infected with highly pathogenic respiratory viruses gets worse after one week or ten days of the infection, progressed to an acute respiratory distress syndrome (ARDS) or acute respiratory diseases (ARD) even died at about couple of weeks after the infection. The clinic characteristic has been observed with the 1918 influenza pandemic, 2009H1N1 pandemic, avian H1N5 and H7N9 infection, the infection of the severe acute respiratory syndrome (SARS) virus and the middle east respiratory syndrome (MERS) virus, and the Coronavirus Disease 2019 (COVID-19) virus, the SARS-CoV-2 virus. It has been reported that the over-reacting immune responses as well as the accompany cytokine storm paly a critical role for the systematic multiple organ damages and death of the infections. Currently, there are no effective medicines for the treatment of a serious infection caused by a highly pathogenic respiratory virus.

Vaccines are the most effective approach to prevent infectious diseases. However, vaccines are not perfect as they may cause serious adverse reactions even death. For example, the swine influenza vaccine in 1976 might be related to about 500 cases of Guillain-Barre syndrome (GBS) and 25 deaths that the vaccine had to be called off (US CDC, VAERS). The 2009 monovalent H1N1 (swine) influenza vaccine might have induced 636 serious health events, including 103 cases of GBS and 51 deaths in the United States (US CDC, VAERS). The vaccination of the COVID-19 virus might have induced 2,509 deaths in the United States among people who received a COVID-19 vaccine (0.0017%) during Dec. 14, 2020, through Mar. 29, 2021 (US CDC, VAERS). Thus far, there are not any medicines for preventing and treating the serious adverse reactions of influenza vaccines or other vaccines due to the unclear pathogenic mechanisms.

Therefore, there remains a need for novel effective therapeutics useful in the treatment, and prevention of infectious diseases particularly for a pandemic caused by highly pathogenic viruses. In particular, there remains a need for effective medicines for the prevention and treatment of the over-reacting immune responses and the cytokine storm. Also, there remains a need for therapeutics in the improvement of vaccine's safety for a better control of infectious diseases particularly for a pandemic caused by highly pathogenic viruses.

Sialic acid is a generic term for the N- or O-substituted derivatives of neuraminic acid, a nine-carbon monosaccharide. Certain sialic acids such as N-Acetylneuraminic acid as a component of viral receptors are related to viral attachment of influenza viruses or coronavirus. (James Stevens et al. 2006. Science, Vol 312; Xinchuan Huang, et al. 2015. J Virology, Vol 89). Sialic acid has a negative charge on its surface, it is not easy to enter cells and is metabolized rapidly in vivo. Therefore it is difficult for sialic acid alone to be used as a clinic therapeutic.

An analog or derivative of N-Acetylneuraminic acid is N-Acetylneuraminic acid methyl ester which is widely used in the fields of medical therapeutics, agricultural drugs and chemical industry. It is easier for N-Acetylneuraminic acid methyl ester to enter cells compared with N-Acetylneuraminic acid. PCT/US2009/039810 (Glycan-based molecular mimicry array and the uses thereof) described compositions comprising N-acetylneuraminic acid and N-Acetylneuraminic acid methyl ester, for the treatment and prevention of infectious diseases; PCT/US2014/25918 (Biological therapeutics for infectious or inflammatory diseases or conditions) described the compositions comprising biological therapeutics such as immunoglobulin products, serum or plasma and N-acetylneuraminic acid or N-Acetylneuraminic acid methyl ester or both, for the treatment and prevention of the infections caused by viral pathogens. PCT/US2014/25926 (Compositions and products for infectious or inflammatory diseases or conditions) described compositions comprising N-acetylneuraminic acid and N-acetylneuraminic acid methyl ester for the treatment and prevention of infectious diseases, or gastrointestinal and respiratory disorders, or respiratory disorders.

However, the inventor further found that N-acetylneuraminic acid methyl ester was unstable and it is difficult to achieve a sustained efficacy when N-acetylneuraminic acid methyl ester was used alone as a therapeutic.

All references cited herein, including patent applications, patent publications, and UniProtKB/Swiss-Prot Accession numbers are herein incorporated by reference in their entirety, as if each individual reference were specifically and individually indicated to be incorporated by reference.

### BRIEF SUMMARY

To meet the demand for effective therapeutics useful in the treatment, and prevention of infectious diseases particularly for a pandemic caused by highly pathogenic viruses, and to improve the stability and efficacy of the N-acetylneuraminic acid methyl ester, disclosed herein are the improved formulations or the products or compositions for the treatment and/or prevention of saccharide related diseases.

The inventor found that the N-acetylneuraminic acid methyl ester is more stable under the pH conditions of 3.0-6.8, preferably 3.5-6.0, preferably 4.0-5.5, most preferably 4.5-5.0, as shown in the Exemplification, FIGS. 1-2 and FIG. 5. This improvement made it possible for N-acetylneuraminic acid methyl ester to be used independently as a therapeutic for the treatment and/or prevention of saccharide related diseases, as shown in the Exemplification and FIGS. 6-12. In addition, the inventor further found that the stability of N-acetylneuraminic acid methyl ester was better when it was in a composition comprising N-acetylneuraminic acid under certain ratio range (1-5:1) of the two compounds. Further, the efficacy of the composition was better than the previous compositions disclosed in the previous applications, as shown in the Exemplification and FIGS. 6-12.

Therefore, in one aspect, the present disclosure provides the compositions or the products comprising N-acetylneuraminic acid methyl ester. In some embodiments, the pH of the compositions or the products when dissolved (e.g., in an aqueous solution or buffer) is between 3.0-6.8. In further embodiments, the pH of the compositions or the products when dissolved is between 3.5-6.0, preferably 4.0-5.5, most preferably 4.5-5.0. In some embodiments, the N-Acetylneuraminic acid methyl ester comprises the formula of C₁₂H₂₁NO₉, or the molecular structure of:

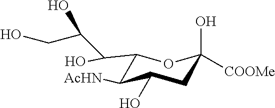

In another aspect, the present application discloses the compositions or the products comprising N-acetylneuraminic acid methyl ester and at least one of the additives of the present disclosure. In some embodiments, the additives of the present disclosure include but not limited to N-acetylneuraminic acid, sodium citrate or sodium acetate, and/or any other applicable articles known in the ordinary arts. In certain embodiments, the compositions or the products comprise N-acetylneuraminic acid methyl ester and N-acetylneuraminic acid. In some embodiments, the ratios of N-acetylneuraminic acid methyl ester to N-acetylneuraminic acid are 1-5:1, preferably 1.25-4:1, most preferably 2-4:1. In some embodiments, the therapeutic products comprise N-acetylneuraminic acid methyl ester, N-acetylneuraminic acid, and sodium citrate or sodium acetate. In some embodiments, the ratios of N-acetylneuraminic acid methyl ester to N-acetylneuraminic acid to sodium citrate or sodium acetate are 1-5:1:0.25-1, preferably 1.25-4:1:0.25-1, most preferably 2-4:1:0.25-1. In some embodiments, when dissolved, the pH of the compositions or the products of any of the preceding embodiments is between 3.0-6.8, preferably 3.5-6.0, preferably 4.0-5.5, most preferably 4.5-5.0.

In another aspect, provided herein are the compositions or the products or the therapeutic medicines comprising N-acetylneuraminic acid methyl ester and methionine. In certain embodiments, the compositions or the products or the therapeutic medicines comprise N-acetylneuraminic acid and methionine. In certain embodiments, the compositions or the products or the therapeutic medicines comprise N-acetylneuraminic acid, N-acetylneuraminic acid methyl ester and methionine. In certain embodiments, the compositions or the products or the therapeutic medicines comprise N-acetylneuraminic acid, N-acetylneuraminic acid methyl ester, methionine and sodium citrate or sodium acetate. In certain embodiments, the pH of the compositions or the products or the therapeutic medicines when dissolved is between 3.0-6.8. In further embodiments, the pH of the compositions or the products or the therapeutic medicines when dissolved is between 3.5-6.0, preferably 4.0-5.5, most preferably 4.5-5.0. In further embodiments, the ratios of N-acetylneuraminic acid methyl ester or N-acetylneuraminic acid to methionine are 0.2-1:1, preferably 1:1. In further embodiments, the ratios of N-acetylneuraminic acid methyl ester to N-acetylneuraminic acid to methionine are 0.2-1:0.2-1:1-2 (N-acetylneuraminic acid methyl ester:N-acetylneuraminic acid:methionine), preferably 1:1:2. In further embodiments, the ratios of N-acetylneuraminic acid methyl ester to N-acetylneuraminic acid to methionine to sodium citrate or sodium acetate of the compositions or the products or the therapeutic medicines are 0.2-1:0.2-1:1-2:0.25-1 (N-acetylneuraminic acid methyl ester:N-acetylneuraminic acid:methionine:sodium citrate or sodium acetate), preferably 1:1:2:1.

Another aspect of the present application is to discloses the compositions or the products comprise at least one of the analogs or derivatives of N-acetylneuraminic acid. In some embodiments, the analogs or derivatives of N-acetylneuraminic acid comprise the general chemical structure of

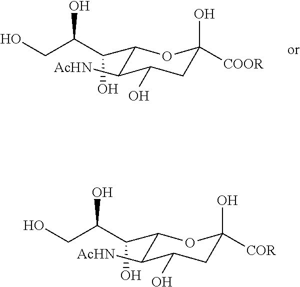

In some embodiments, R is a hydroxyl, hydrogen, alkoxy, alkyl, cycloalkyl, sodium (Na), substituted alkyl, substituted cycloalkyl, aryl, or substituted aryl, ether, HN, H₂N, NHAc, thioester, S—CH₂—CH₃, disulfide ester, S—CH₃, disulfide methyl, methionine, methionine-zinc or phenol or phenol derivatives. In some embodiments, when dissolved the pH of the compositions or the products comprising at least one of the analogs or derivatives of N-acetylneuraminic acid is between 3.0-6.8, preferably 3.5-6.0, preferably 4.0-5.5, most preferably 4.5-5.0.

In some aspects, the compositions or the products of any of the preceding embodiments are used for the treatment and/or prevention of saccharide related diseases. In some embodiments, the saccharide related diseases include but not limited to infectious diseases, infection-relating diseases, complications and sequela of infections, COVID-19 long haulers, cytokine storm and cytokine release syndrome (CRS), adverse reactions of vaccines or therapeutic antibodies, inflammation, inflammatory respiratory diseases, inflammatory gastrointestinal diseases, autoimmune diseases, allergy and cancers; preferably a saccharide related disease caused by a pathogenic pathogen or vaccines relating to the pathogen. The saccharide related diseases also include but not limited to abortion, postpartum labor, still birth of pregnant females, neonatal death and neonatal sudden death, caused by an infection or by a vaccine.

In some embodiments, the saccharide related diseases are caused by an infectious pathogen or vaccines relating to the pathogen. In some embodiments, the saccharide related diseases are caused by pathogenic antibodies inducible by an infectious pathogen or the vaccines relating to the pathogen. In some embodiments, the saccharide related diseases are caused by bacteria or vaccines relating to the bacteria. In some embodiments, the saccharide related diseases are caused by pathogenic antibodies inducible by bacteria or the vaccines relating to the bacteria. In some embodiments, the saccharide related diseases are caused by a virus or vaccines relating to the virus. In some embodiments, the saccharide related diseases are caused by pathogenic antibodies inducible by a virus or the vaccines relating to the virus. In some embodiments, the saccharide related diseases are caused by a virus or vaccines relating to the virus. In some embodiments, the saccharide related diseases are caused by pathogenic antibodies inducible by a virus or the vaccines relating to the virus. In some embodiments, the saccharide related diseases are caused by a respiratory virus, or vaccines relating to the respiratory virus. In some embodiments, the saccharide related diseases are caused by pathogenic antibodies inducible by the respiratory virus or the vaccines relating to the respiratory virus. In some embodiments, the saccharide related diseases are caused by an enterovirus, or vaccines relating to the enterovirus. In some embodiments, the saccharide related diseases are caused by pathogenic antibodies inducible by an enterovirus or the vaccines relating to the enterovirus. In some embodiments, the saccharide related diseases are caused by pathogenic antibodies inducible by the enterovirus or the vaccines relating to the enterovirus.

In some embodiments, the respiratory viruses include but not limited to influenza viruses, respiratory enterovirus, adenovirus, coronavirus, rhinovirus, respiratory syncytial virus or B virus. In some embodiments, the respiratory viruses include but not limited to influenza viruses, comprising type A, type B and type C influenza viruses. In some embodiments, the influenza A viruses include but not limited to H1N1, H3N2, H5N1, H7N9, H7N8, or H9N2 virus, and any variants or newly emerging strains of the influenza viruses. In some embodiments, the coronaviruses include but not limited to the SARS-CoV-2 virus, SARS-CoV viruses, MERS-CoV viruses, avian infectious bronchitis virus (IBV), avian coronavirus, and any variants or newly emerging strains of the viruses. In some embodiments, the enteroviruses include but not limited to rotavirus, reovirus, Coxsackie virus, Echoviruses, Enteroviruses, Polioviruses, norovirus, coronavirus, Norwalk virus, cytomegalovirus (CMV), herpes simplex virus, hepatitis virus, enteric cytopathic human orphan (ECHO) virus, porcine enterovirus (PEV), transmissible gastroenteritis virus (TGEV), foot and mouth disease (HFMD), human enterovirus 71, and porcine epidemic diarrhea virus (PEDV).

In some embodiments, the saccharide related diseases include but not limited to complications and sequela induced during or after infections of the infectious pathogens described in any of the preceding embodiments. In some embodiments, the sequela includes COVID-19 long haulers. In some embodiments, the complication and sequela include but not limited to inflammation and injuries of respiratory system, digestive system, cardiovascular system, liver, brain or neural system, kidney and other organs. In some embodiments, the complication induced by the respiratory viruses include but not limited to the acute respiratory distress syndrome (ARDS) or acute respiratory diseases (ARD), cytokine storm and cytokine release syndrome (CRS). In some embodiments, the complication and sequela include but not limited to abortion, postpartum labor, still birth of pregnant females, and neonatal death and neonatal sudden death, caused by an infection, or by a vaccine, or by a pathogenic antibody.

In certain aspects, the present application is to disclose the forms of the compositions or the products of any of the preceding embodiments. In some embodiments, the compositions or the products are in a form of a pulvis, a tablet, a capsule (each including timed release and sustained release formulations), a pill, a powder mixture, a granule, an elixir, a tincture, a solution, a suspension, a syrup or a emulsion, a nasal drop or spray, an injectable, an infusion, or a form conjugated to a nano-particle, or other using forms well known to those of ordinary skill in the relevant arts.

In some embodiments, the compositions or the products of any of the preceding embodiments are used as therapeutic products, dietary supplement products, food, feed, food additives, feed additives, therapeutic products, rehydration salt or rehydration solution, and any other applicable uses.

In another aspect, the present invention discloses the methods of using the compositions or the products of any of the preceding embodiments for the treatment and/or prevention of saccharide related diseases, comprising administering an effective amount of at least one of the compositions or the products of any of the preceding embodiments to a patient or an individual suffering or developing a saccharide related disease described in any of the preceding embodiments.

In some embodiments, the effective dosages of the compositions or the products for the uses as mentioned in the preceding embodiments are from about 0.001 mg/kg to about 100 mg/kg of a sialic acid (e.g. N-acetylneuraminic acid), or an analog or derivative of a sialic acid (e.g. N-acetylneuraminic acid methyl ester), or an additive (e.g. sodium citrate or sodium acetate).

In some embodiments, the administrating routes of a therapeutic product include but not limited to subcutaneous, topical with or without occlusion, oral, intramuscular, intravenous (both bolus and infusion), intraperitoneal, intracavity, or transdermal, inhalant, or other using forms well known to those of ordinary skill in the pharmaceutical arts.

In some embodiments, the saccharide related diseases include but not limited to any of the preceding embodiments. In some embodiments, the saccharide related diseases include infectious diseases, infection-relating diseases, complications and sequela of infections, COVID-19 long haulers, cytokine storm and cytokine release syndrome (CRS), adverse reactions of vaccines or therapeutic antibodies, inflammation, inflammatory respiratory diseases, inflammatory gastrointestinal diseases, autoimmune diseases, allergy and cancers. In some embodiments, a saccharide related disease is caused by a pathogen or vaccines relating to the pathogen. In some embodiments, a saccharide related disease is caused by pathogenic antibodies inducible by pathogens or vaccines relating to the pathogens. In some embodiments, the saccharide related diseases also include but not limited to abortions, postpartum labors, still births of pregnant females, neonatal death and neonatal sudden death, caused by an infection, or by a vaccine, or by a pathogenic antibody.

In some embodiments, the patients include but not limited to humans or animals. In some embodiments, the humans include but not limited to males and females, newborns, 1-12 months old infants, 1-18 years old children, adults, old people, pregnant and feeding females. In some embodiments, the animals include but not limited to livestocks including but not limited to cows, pigs, horses, sheep or goats, llamas, cattle, donkeys; poultry including but not limited to chickens, ducks, gooses, turkeys and pigeons; companion animals including but not limited to dogs, cats, rodent pets and avian pets. More specifically, the livestocks include but not limited to males and females, adult animals, newborn animals, infant animals, and other young age animals, pregnant and feeding female animals.

In another aspect, the present invention discloses the methods of making the compositions or the products for the treatment and/or prevention of saccharide related diseases of any of the preceding embodiment, comprise preparing a suitable amount of an analog or a derivative of a sialic acid alone or the analog of a sialic acid in conjunction with at least one of the other major components of the present disclosure. The major components of the present disclosure include but not limited to: 1) a derivatives or an analogs of a sialic acid (e.g.

N-Acetylneuraminic acid methyl ester) 2) a sialic acids including but not limited to N-acetylneuraminic acid, 2-Keto-3-deoxynononic acid, N-Acetylglucosamine, N-Acetylgalactosamine, N-Acetylmannosamine, and N-Glycolylneur-aminic acid; 3) another saccharides including but not limited to fructose, glucose, mannose, fucose, xylose, galactose, lactose; 4) a saccharide modifying molecules including but not limited to sulfur-containing amino acids (e.g. methionine and methionine-zinc complex); and 5) nutritional or pharmaceutically acceptable salts include but not limited to sodium chloride, potassium chloride, sodium citrate, sodium acetate, or the oral rehydration salts recommended by WHO. In some embodiments that may be combined with any of the preceding embodiments, the pH of the compositions or the products or the therapeutic medicines is between 3.0-6.8, preferably 3.5-6.0, preferably 4.0-5.5, most preferably 4.5-5.0.

The current invention with in vitro and in vivo supporting data further discloses a new mechanisms of pathogenesis (MOP) of an highly pathogenic respiratory viral infection. The MOP include: 1) a highly pathogenic respiratory viruse such as the COVID-19 virus or the avian influenza virus causes the initial, primary damage (e.g. removing sialic acid from cell surface) of its target organ (e.g. lung); 2) certain antibodies (e.g. anti-SARS-CoV-2 spike antibody) induced by the virus can bind to the damaged and the inflammatory cells (with missing sialic acid) of the target organ (e.g. lung) (FIG. 13) and other organs (e.g. heart, brain and kidney) (FIG. 14 and FIG. 17), mislead the immune response to attack the self cells or tissues and induces further damage (secondary damage); 3) the secondary damage can persistently add further damage to the primary damage and cause serious conditions (e.g. ARDS) even death as the antibodies elevete and reach the peak levels from week two to week three or four. 4) the overreacting immune responses (e.g. cytokine storm) misleaded by the pathogenic antibodies can be persistent and accumilated after viral clearance whenever the antibody exist.

The primary damage can be limited, short and decreased as the virus being cleared (such as a regular influenza infection). That means the virus itself is not enough to cause a serious condition such as ARDS, or cytokine stor, or death. However, the secondary damage caused by the pathogenic antibodies can be longer and broader because antibodies persist much longer than viruses and can bind nonspecifically to other inflammatory tissues besides lung. The new MOP can explain why most patients with serious respiratory viral infections such as COVID-19 or avian influenza infection died after one week especially at 2-4 weeks, matching the period of antibody peak levels. The new MOP of an highly pathogenic viral infection can also explan the serious adverse reactions observed with the vaccines of respiratory viruses such as the COVID-19 virus and the influenza viruses. Similarly, certain pathogenic antibodies inducible by other infectious pathogens or other vaccines may also cause serious adverse reactions or autoimmune diseases through the similar pathogenic mechanism, and cancers if the inflammatory cellular proliferation loses control.

The current invention not only discloses the new MOP which is responsible for the death of a highly pathogenic viral infection (e.g. COVID-19 infection) or the serious adverse reaction of the vaccines related to the pathogen (e.g. COVID-19 vaccines), but also provides a therapeutic for the prevention and treatment of the diseases.

Therefore, in one embodiment, the present invention discloses a unique function of N-acetylneuraminic acid methyl ester. As shown in the Exemplification and FIG. 3D, N-acetylneuraminic acid methyl ester is helpful for the repairment of missed sialic acid on the damaged lung epithelium A549 cells. The structure recovery of the damaged cells can reduce the self-attack of immune system to the damaged or inflammatory cells and prevent the damage of lung and other organs (Mechanism of Action, MOA-1). Moreover, the replacement of N-acetylneuraminic acid by N-acetylneuraminic acid methyl ester could induce a structural or chemical modification of the viral receptor that significantly decreased the binding affinity of the SARS-CoV-2 viral receptor binding domain (MOA-2) (FIG. 4).

Based on the MOA-2, N-acetylneuraminic acid methyl ester can prevent the COVID-19 infection by blocking viral entry into host cells, and treat the infection by blocking viral spreading into new cells. More importantly, the structure repairment of the damaged cells by N-acetylneuraminic acid methyl ester can reduce the self-attack of immune responses and prevent the systemic damage of lung and other organs (MOA-1). Because sialic acid is a receptor for not only coronavirus but also other viruses (e.g. influenza viruses or rotavirus) the receptor modification and blocking entry, and the structural repairment by N-acetylneuraminic acid methyl ester should be widely effective for the prevention and treatment of other infections of other viruses using sialic acids as receptors (e.g. influenza viruses or rotavirus).

As a support, the data of in vivo study of influenza viral infections indicated that compositions comprising N-acetylneuraminic acid methyl ester could effectively inhibit an over-reacting immune response by significantly reducing the damage of lung and other organs, decrease the death rate, and reducing cytokine release, as shown in the Exemplification and FIGS. 5-9, and can be widely effective for other infectious diseases preferably caused by highly pathogenic viruses (e.g. COVID-19) and their antibodies as described in the current disclosure.

As another support, the data of in vivo study also indicated that the compositions or the products comprising N-acetylneuraminic acid methyl ester are effective for the prevention and treatment of rheumatoid arthritis, as shown in the Exemplification and FIG. 10; and can be widely effective for other autoimmune diseases preferably caused by pathogenic antibodies and/or missing sialic acid as described in the current disclosure.

As further support, the pethogenic antibodies can bind to the unmaturedunmatured fetal cells or tissues (FIG. 16) and cause abortions, postpartum labors, still births of pregnant females, and neonatal deaths and neonatal sudden deaths, as shown in the Exemplification and FIGS. 11-14.

As further support, the pathogenic antibodies can bind to the human inflammatory disease tissues or cancer tissues of respiratory, cardiovesvular, urinary, and digestive system (FIG. 17A-B), and cause serious infections preferably highly pathogenic viral infections (e.g. COVID-19 infection), serious adverse reactions of vaccines (e.g. COVID-19 vaccines), serious complications of infections (e.g. ARDS), infection-relating inflammation and autoimmune diseases, and infection-relating cancers which can occur if an inflammatory cellular proliferation stimulated by an pathogenic antibody repeatedly persists for long time and loses control.

Thus the compositions or the products comprising N-acetylneuraminic acid methyl ester of the current disclosure are widely effective for the treatment and prevention of the diseases or conditions caused by pathogenic antibodies as described in any of the preceding embodiments. The pothogenic antibodies can be induced by a pothogen partivularly a highly pathogenic virus (e.g. the SARS-CoV-2 virus), a vaccine partivularly a vaccine of a highly pathogenic virus (e.g. a COVID-19 vaccine), or a therapeutic antibody.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIGS. 1A-1D show the stability of N-acetylneuraminic acid (NANA; FIG. 1A) or N-acetylneuraminic acid methyl ester (NANA-Me; FIG. 1A) at different pH conditions, and the composition of NANA and NANA-Me with the ratio of 1:1 (FIGS. 1C & 1D).

FIGS. 2A-2C show the remaining levels of NANA-Me of the compositions consisted of NANA and NANA-Me with different ratios at the conditions of pH 6.0-7.0 (FIGS. 2A & 2B), or pH 7.3 (FIG. 2C).

FIGS. 3A-3D show the levels of sialic acid on the A549 cells treated with the N-acetylneuraminic acid (NANA) (FIG. 3A) or N-acetylneuraminic acid methyl ester (NANA-Me) (FIG. 3B) at various concentrations, and the compositions consisted of 50 μg/ml of NANA-Me in combination with NANA at different ratios (FIG. 3C). FIG. 3D shows the levels of sialic acid on the A549 cells with or without the treatment of sialidase and a composition consisted of NANA-Me and NANA (BH-103, pH 4.5).

FIGS. 4A-4F show the levels of sialic acid on the NB4 cells with or without the treatment of the formulation of BH-103.3 (shown as BH-103) (FIG. 4A) or with sialidase and BH-103.3 (FIG. 4C); FIG. 4B shows the levels of the receptor binding domain (RBD) of the spike (S) protein of the SARS-CoV-2 virus bound to NB4 cells with or without BH-103.3 treatment; FIG. 4D shows the S-RBD-binding NB4 cells with or without the treatment of sialidase and BH-103.3. FIG. 4E shows the S-RBD-binding HEK-293 cells with or without BH-103.3 treatment FIG. 4F shows the levels of S-RBD bound to HEK-293 cells with or without BH-103.3 treatment FIGS. 5A & 5B show a mouse model of H1N1 influenza viral infection. FIG. 5A shows the survival rates of mice treated with the compositions consisted of N-acetylneuraminic acid methyl ester (NANA-Me) at pH 1.5 or pH 7.0 and NANA-Me plus NANA (combo) at pH 5.0 and 30 mg/kg, or at pH 6.0 and 15 mg/kg. FIG. 5B shows the survival rates of mice treated with the compositions consisted of NANA-Me at pH 3.5 and 15 mg/kg or at pH 4.5 and 15 mg/kg, or NANA-Me plus NANA (combo) at pH 3.6 and 15 mg/kg.

FIGS. 6A & 6B show a mouse model of H1N1 influenza viral infection. FIG. 6A shows the survival rates (top) and body weight (bottom) of mice treated with Tamilu or the formulation of BH-103.1 (pH 4.5) at 4 hours post viral challenge and 30 mg/kg. FIG. 6B shows the survival rates (top) and body weight (bottom) of mice treated with Tamilu or the formulation of BH-103.1 (pH 4.5) at 24 hours post viral challenge and 15 mg/kg.

FIG. 7 shows the histological changes of lungs and intestines from the mice of the same model of H1N1 influenza infection as shown in FIGS. 6A & 6B.

FIGS. 8A & 8B show the levels of IL-1β, TNF-β and IL-6 cytokines in mouse sera collected at day 6 post infection (FIG. 8A), and the tissue lysates of mouse lungs collected at day 14 (FIG. 8B) from the mice of the same model of H1N1 influenza infection as shown in FIGS. 6A & 6B FIGS. 9A & 9B show a mouse model of H3N2 influenza viral infection. The survival rates (FIG. 9A), healthy score (FIG. 9B) and body weight (FIG. 9C) of mice treated with Tamiflu or the formulation of BH-103.2 (pH 4.5) (BH-103 in FIG. 9) via either orally (PO) or intraperitoneal injection (IP) at 8 hours post viral challenge and each 30 mg/kg.

FIGS. 10A-10C show a collagen-induced arthritis (CIA) model of rats. FIG. 10A shows representative gross images taken at day 5 (after 2 dosing); FIG. 10B shows the paw swollen volume; and FIG. 10C shows the body weight.

FIGS. 11A-11C show a timed-pregnant mouse model and the procedure of injection of anti-coronavirus antibodies into the timed-pregnant mice (FIG. 11A); the representative images of mouse pups delivered to the dames (FIG. 11B); the sick and death rates of newborn mouse pups caused by the pathogenic anti-coronavirus antibodies, and the therapeutic effect of the formulation of BH-103.3 (BH-103 in FIG. 11) (FIG. 11C).

FIG. 12 shows the representative images of the histological changes of lungs (top 2 rows), kidneys, brains and hearts (bottom row) from the newborn mouse pups delivered to the dames injected with antibodies specific to the spike one protein of either SARS-CoV-2 (Anti-COVID-19 S1 in FIG. 12) or SARS-CoV (Anti-SARS S in FIG. 12) virus, and control antibodies including human IgG and human monoclonal antibody (MAb) of Cr3022-b6; or the dames treated in combination with BH-103.3 at the same time of the antibody injection of anti-coronavirus antibodies.

FIGS. 13A & 13B show the detection of anti-coronavirus spike antibodies bound in vivo at the inflammatory areas of the multiple organs of the mouse newborns delivered to the dames with antibody injection at E15 and E18.

FIG. 14 shows the cytokine levels of MCP-1 and IL-4 in mouse sera from the newborn mouse pups delivered to the dames with antibody injection alone of the anti-coronavirus antibodies or the dames treated in combination with BH-103.3 (BH-103 in FIG. 14) at the same time of the antibody injection of anti-coronavirus antibodies.

FIGS. 15A-15E show the binding of anti-coronavirus spike antibodies and anti-influenza viral antibodies to the healthy or damaged human lung epithelium A549 cells with missed sialic acid on the cell surface.

FIG. 16 shows the binding of an human monoclonal anti-COVID-19 S1 antibody of Regn10987 to various human fetal tissues.

FIGS. 17A & 17B show the binding of an human monoclonal anti-COVID-19 S1 antibody of Regn10987 to various human diseased tissues of respiratory, cardiovesvular and urinary system (FIG. 17A), and digestive system (FIG. 17B).

FIGS. 18A-18C show a chicken embryo model of the H9N2 infection. FIG. 18A shows positive infection rates of chicken embryos; FIG. 18B shows the average viral titers at 48 hours post infection; and FIG. 18C shows the viral titers of each embryo at 24 and 48 hours post infection.

FIGS. 19A-19C show a chicken model of avian coronavirus, avian infectious bronchitis virus (IBV) infection.

FIG. 19A shows the survival rates, FIG. 19B shows the body weight and FIG. 19C shows the viral loads of chicks treated with vehicle (saline) or the formulation of BH-103.6 (pH 4.5) at either about 2.5 days previous (30 mg/kg, IP) or 4 hours post viral challenge (50 mg/kg, IP).

### DETAILED DESCRIPTION

The present disclosure provides formulations or products or compositions comprising analogs or derivatives of N-acetylneuraminic acid with a particular pH range. Multiple such formulations or products or compositions are demonstrated herein to treat one or more symptoms of a saccharide related disease in a variety of in vivo models. In particularly, these formulations or products or compositions were found to have increased stability and efficacy as compared to previous formulations or compositions, e.g., in mitigation of inflammation and organ damages as well as decrease of death rate in highly pathogenic respiratory viral infections such as COVID-19 infection, or other diseases related to other viral infections. In addition, these formulations or products or compositions were demonstrated to be effective to the serious adverse reactions of the vaccines of the highly pathogenic respiratory viruses such as the vaccines of the SARS-CoV-2 virus (the cause of COVID-19), representing a range of different disease types.

### General Techniques

The techniques described or referenced herein are well understood and employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); A Laboratory Manual, and Animal Cell Culture (R. I. Freshney, ed. (1987)); Methods in Molecular Biology, Humana Press; or following the manufacturer's instructions.

### I. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

As used herein, the term “saccharide” refers to a monosaccharide, an oligosaccharide or a polysaccharide. Monosaccharides include but not limited to fructose, glucose, mannose, fucose, xylose, galactose, lactose, N-acetylneuraminic acid, N-acetyl-galactosamine, N-acetylglucosamine, and sialic acids. An oligosaccharide is a saccharide polymer containing multiple sugar monomers linked by glycosidic linkages of component sugars.

Proteins are modified by the addition of saccharides, a process termed “protein glycosylation”. Glycoproteins or proteosaccharides refer to proteins linked with saccharides and may typically contain, for example, O- or N-glycosidic linkages of monosaccharides to compatible amino acid side chains in proteins or to lipid moieties. As used herein, the terms “glycan” and “glycosylmoiety” may be used interchangeably to refer to a saccharide alone or a sugar as the saccharide component of a glycoprotein. Two types of glycosylation are known in the art: N-linked glycosylation to the amide nitrogen of asparagine side chains and O-linked glycosylation to the hydroxy oxygen of serine and threonine side chains. Other saccharides include but not limited to O-GlcNAc, GAG Chain, glycosaminosaccharides, and glycosphinglipid. O- and N-linked saccharides are very common in eukaryotes but may also be found, although less commonly, in prokaryotes.

While many proteins are known to be glycosylated, glycoproteins are often found on the exterior surface of cells (i.e., extracellular) or secreted. Because of this, glycoproteins are highly accessible to external agents (e.g., exogenous compounds administered to a patient). For example, components that specifically recognize certain glycoproteins (e.g., antibodies or lectins) are able to bind, to an intact organism, to cells that express these glycoproteins on their cell surface. Components that specifically recognize certain glycoproteins are also able to bind secreted saccharides or glycoproteins, for example those that may be found freely in certain tissue samples (including, without limitation, in blood or serum).

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. An individual is successfully “treated”, for example, if one or more symptoms associated with cancer are mitigated or eliminated.

As used herein, the term “prevention” includes providing prophylaxis with respect to occurrence or recurrence of a disease in an individual. An individual may be predisposed to, susceptible to a type of cancer, or at risk of developing a type of cancer, but has not yet been diagnosed with the disease.

An “effective amount” refers to at least an amount effective, at dosages and for periods of time necessary, to achieve the desired therapeutic or prophylactic result. An effective amount can be provided in one or more administrations.

A “therapeutically effective amount” is at least the minimum concentration required to effect a measurable improvement of a particular disorder (e.g., cancer). A therapeutically effective amount herein may vary according to factors such as the disease state, age, sex, and weight of the patient, and the ability of the monoclonal antibody to elicit a desired response in the individual. A therapeutically effective amount is also one in which any toxic or detrimental effects of the monoclonal antibody are outweighed by the therapeutically beneficial effects. A “pophylactically effective amount” refers to an amount effective, at the dosages and for periods of time necessary, to achieve the desired prophylactic result. Typically but not necessarily, since a prophylactic dose is used in subjects prior to or at an earlier stage of disease, a prophylactically effective amount may be less than a therapeutically effective amount.

As used herein, administration “in conjunction” with another compound or composition includes simultaneous administration and/or administration at different times. Administration in conjunction also encompasses administration as a co-formulation or administration as separate compositions, including at different dosing frequencies or intervals, and using the same route of administration or different routes of administration.

An “individual” for purposes of treatment or prevention refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. In some embodiments, the individual is human. In some embodiments, the individual is a non-human animal.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; low molecular weight (less than about 10 residues) polypeptide; proteins, such as serum albumin, gelatin, or immunoglobulins; amino acids such as glycine, glutamine, asparagine, arginine or lysine; carbohydrates including glucose, mannose, or dextrin; and/or nonionic surfactants such as TWEEN™, polyethylene glycol (PEG), and PLURONICS™.

“Pharmaceutically acceptable” buffers and salts include those derived from both acid and base addition salts of the above indicated acids and bases. Specific buffers and/or salts include histidine, succinate and acetate.

“Dehydration” is defined as the excessive loss of body fluid. “Rehydration” is defined as the correction of a dehydrated state by the replenishment of electrolytes through oral rehydration therapy or fluid replacement by intravenous therapy. “Intravenous rehydration” refers to the replenishment of electrolytes by intravenous therapy.

“ORT” refers to oral rehydration therapy. “ORS” refers to oral rehydration solution or salt.

### II. Components of Compositions or Products

### Major Components

The major component of the compositions or products of the present disclosure include but not limited to the analogs or derivatives sialic acid, other saccharides, saccharide modifying molecules.

Sialic acid A sialic acid (Sia) is a generic term for the N- or O-substituted derivatives of neuraminic acid, a nine-carbon monosaccharide. It is also the name for the most common member of this group, N-acetylneuraminic acid (Neu5Ac or NANA) and 2-Keto-3-deoxynononic acid (Kdn). Other members of sialic acids include but not limited to N-Acetylglucosamine (GlcNAc), N-Acetylgalactosamine (GalNAc), N-Acetylmannosamine (ManNAc), and N-Glycolylneur-aminic acid (Neu5Gc). The amino group bears either an acetyl or a glycolyl group as described below. N-acetylneuraminic acid can be used as an acidic reagent to achieve a desired pH value of a composition or a product solution. Sialic acids are found widely distributed in human or animal tissues, especially in glycoproteins and gangliosides. N-acetylneuraminic acid can be either isolated from natural materials or artificially synthesized with following characteristics.

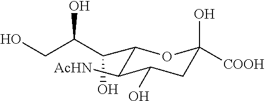 [SMILES: CC(=O)NC1C(O)CC(O)(C(=O)O)OC1[C@H](O)[C@H](O)CO]

Molecular formula: C₁₁H₁₉NO₉ molecular weight: 309.3.

### Analogs or Derivatives of Sialic Acids

A major component of the compositions or the compositions or the products of the present disclosure comprises any molecules having the general chemical structure of

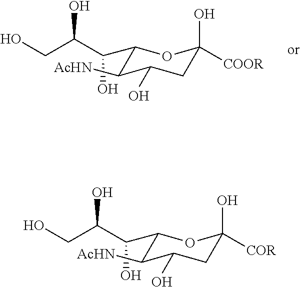

wherein R is a hydroxyl, hydrogen, alkoxy, alkyl, cycloalkyl, sodium (Na), substituted alkyl, substituted cycloalkyl, aryl, or substituted aryl, ether, HN, H₂N, NHAc, thioester, S—CH₂—CH₃, disulfide ester, S—CH₃, disulfide methyl, methionine, methionine-zinc or phenol or phenol derivatives. In some embodiments, when dissolved the pH of the compositions or the products comprising at least one of the analogs or derivatives of N-acetylneuraminic acid is between 3.0-6.8, preferably 3.5-6.0, preferably 4.0-5.5, most preferably 4.5-5.0.

Another major component of the compositions or compositions or products of the present disclosure comprises any other applicable analogs or derivatives of sialic acids with the R at the position of other eight carbons of the above structure; and/or any other relevant or similar molecules of sialic acids, or any other forms of sialic acids identified as the active ingredient.

The hydroxyl substituents of sialic acids may vary considerably: acetyl, lactyl, methyl, sulfate and phosphate groups have been found. The other hydroxyl substituents of sialic acids include but not limited to crotonyl-, succinyl-, propionyl-, butyryl- and sulfur-groups. One example of an analog of N-Acetylneuraminic acid is N-Acetylneuraminic acid methyl ester as shown below.

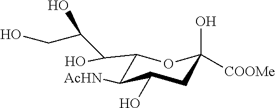

Molecular weight: 323.3, formula: C₁₂H₂₁NO₉.

In a formulation or a product or a composition, an analog or a derivative of sialic acids including N-Acetylneuraminic acid methyl ester can be included alone or in conjunction or in combination with other components of the present disclosure. The pH of the compositions or the products or the compositions when dissolved (e.g., in an aqueous solution or buffer) is between 3.0-6.8, preferably 3.5-6.0, preferably 4.0-5.5, most preferably 4.5-5.0.

The amounts or concentrations of an analog or a derivative of sialic acids including N-Acetylneuraminic acid methyl ester in a formulation or a product or a composition of the present disclosure is from about 0.01 mg/ml to about 900 mg/ml or 0.01 mg/g to about 900 mg/g.

Other saccharides Another major component of the compositions or products of the present disclosure comprises other saccharides beside sialic acids. The term of the other saccharides of the present disclosure refers to a monosaccharide, an oligosaccharide or a polysaccharide. Monosaccharides include but not limited to fructose, glucose, mannose, fucose, xylose, galactose, lactose. An oligosaccharide is a saccharide polymer containing a small number (typically three to ten) of component sugars, also known as simple sugars.

The other saccharides (e.g. a galactose or a lactose) of the present disclosure include but not limited to glucosamine, galactosamine, mannosamine, O-GcNAc, GAG Chain, glycosaminosaccharides, and glycosphinglipid.

In a formulation or a product or a composition, the other saccharides (e.g. a galactose or a lactose or a glucosamine) of the present disclosure can be included alone or in combination with a sialic acid or an analog or derivative of a sialic acid or other components of the present disclosure. The pH of the compositions or the products or the compositions when dissolved is between 3.0-6.8, preferably 3.5-6.0, preferably 4.0-5.5, most preferably 4.5-5.0.

The amounts or concentrations of other saccharides in a formulation or a product or a composition of the present disclosure is from about 0.01 mg/ml to about 900 mg/ml or 0.01 mg/g to about 900 mg/g.

### Saccharide Modification Molecules

Another major component of the compositions or products of the present disclosure comprises saccharide modification molecules. As used herein, saccharide modification molecules refer to molecules containing acetyl-, lactyl-, methyl-, phosphate-, crotonyl-, succinyl-, propionyl-, butyryl- and sulfur-groups as donors for the modification of sialic acids or other saccharides. Other molecules capable of modifying sialic acids or other saccharides in other forms are also included without limitation.

In a formulation or a product or a composition, a saccharide modification molecule of the present disclosure can be included alone or in combination with a sialic acid or an analog or derivative of a sialic acid or other components of the present disclosure. The pH of the compositions or the products or the compositions when dissolved is between 3.0-6.8, preferably 3.5-6.0, preferably 4.0-5.5, most preferably 4.5-5.0.

The amounts or concentrations of a saccharide modification molecule in a composition or a product of the present disclosure is from about 0.01 mg/ml to about 900 mg/ml or 0.01 mg/g to about 900 mg/g.

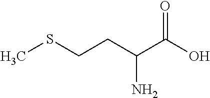

Molecular formula: C₅H₁₁NOₛS, molecular weight: 149.21.

In a formulation or a product or a composition of the present disclosure, methionine can be included alone or in combination with a sialic acid or an analog or derivative of a sialic acid or other components of the present disclosure. The pH of the compositions or the products or the compositions when dissolved is between 3.0-6.8, preferably 3.5-6.0, preferably 4.0-5.5, most preferably 4.5-5.0.

Nutritional or Pharmaceutically Acceptable Additives and/or Salts

Nutritional or pharmaceutically acceptable additives and/or salts include but not limited to minerals, vitamins, sodium chloride, potassium chloride, sodium citrate, sodium acetate, sodium bicarbonate (NaHCO₃), or any other additives or salts known in the ordinary art.

One example is the oral rehydration salts recommended by WHO comprising 3.5 grams of sodium chloride, 1.5 grams of potassium chloride, 2.9 grams of sodium citrate, and 20 grams of glucose, in one liter of water. The amount of potassium citrate or glucose can be adjusted (e.g. reduced). N-acetylneuraminic acid can be used as an acidic reagent to achieve a desired pH value of an ORS.

### Other Therapeutics

Other therapeutics include existing or new therapeutics (known or unknown). Existing or new therapeutics (known or unknown) include but not limited to products consisted of chemicals (e.g. antibiotics), biologicals (e.g. antibodies, proteins and blood products), plants or herbs, and etc. without limitation. Examples of existing or new therapeutics include but not limited to antibiotics or other anti-infective (e.g. interferon or antibodies), anti-inflammation, anti-allergy, anti-autoimmune diseases, anti-oncological diseases, anti-gastrointestinal diseases, anti-respiratory diseases, anti-cardiovascular diseases, anti-neurological diseases, anti-urological diseases, anti-reproductive diseases, anti-endocrine diseases, and any other known or unknown therapeutics without limitation.

### Optional Components

### Plant Isolates

One optional component of the compositions or products of the present disclosure comprises plant isolates. As used herein, a plant isolate refers to any ingredients or molecules isolated from a plant. In a formulation or a product or a composition of the present disclosure, a plant isolate can be included in combination with a sialic acid or an analog or derivative of a sialic acid or other components of the present disclosure. The amounts or concentrations of a plant isolate in a composition or a product of the present disclosure is from about 0.01 mg/ml to about 900 mg/ml or 0.01 mg/g to about 900 mg/g.

### Inorganic Ions

Another optional component of the compositions or the compositions or the products of the present disclosure comprises the inorganic ions. As used herein, inorganic ions include mineral nutrients that include but not limited to elements boron, copper, manganese, zinc, molybdenum, sulphur, iron, calcium, potassium, nitrate, phosphate, chloride, etc., without limitation. In a formulation or a product or a composition of the present disclosure, an inorganic ion can be included in combination with a sialic acid or an analog or derivative of a sialic acid or other components of the present disclosure. The amounts or concentrations of an inorganic ion in a composition or a product of the present disclosure is from about 0.001 mg/ml to about 500 mg/ml or 0.001 mg/g to about 500 mg/g.

### Herbs and Traditional Chinese Herbs

Another optional component of the compositions or the products of the present disclosure comprises herbs. As used herein, an herb refers to a plant that is valued for qualities such as medicinal properties, flavor, scent, or the like. In the present disclosure, traditional Chinese herbs include but not limited to all herbs listed in Bencao Gangmu (simplified Chinese: [symbol] ; pinyin: Běnc\check{a}oGāngmù), also known as Compendium of Materia Medica, which is Chinese materia medica work written by Li Shizhen in Ming Dynasty. It is a work epitomizing materia medica ( [symbol] ) in Ming Dynasty. It lists all the plants, animals, minerals, and other objects that were believed to have medicinal properties.

In a formulation or a product or a composition of the present disclosure, a herb can be included in combination with a sialic acid or an analog or derivative of a sialic acid or other components of the present disclosure. The amounts or concentrations of a herb in a composition or a product of the present disclosure is from about 0.01 mg/ml to about 900 mg/ml or 0.01 mg/g to about 900 mg/g.

### Others

Other optional components include but not limited to sugar, dextrin, starch, salt, gelatin and any other necessary components or materials known in the art.

### III. Compositions or Products

In one aspect, the present invention discloses compositions or products comprise an analog or derivative of a sialic acid alone, or an analog or derivative of a sialic acid plus at least one of the other major or optional components as described in any of the preceding embodiments of the present disclosure.

In one embodiment, provided herein are the compositions or the products comprising N-acetylneuraminic acid methyl ester. In certain embodiments, the pH of the compositions or the products when dissolved is between 3.0-6.8. In further embodiments, the pH of the compositions or the products when dissolved is between 3.5-6.0, preferably 4.0-5.5, most preferably 4.5-5.0. In certain embodiments, the amounts or concentrations of N-Acetylneuraminic acid methyl ester in a formulation or a product or a composition of the present disclosure is from about 0.01 mg/ml to about 900 mg/ml or 0.01 mg/g to about 900 mg/g.

In another embodiment, provided herein are the compositions or the products or the therapeutic medicines comprising N-acetylneuraminic acid methyl ester and N-acetylneuraminic acid. In certain embodiments, the compositions or the products or the therapeutic medicines comprise N-acetylneuraminic acid methyl ester, N-acetylneuraminic acid, and sodium citrate or sodium acetate. In certain embodiments, the pH of the compositions or the products or the therapeutic medicines when dissolved is between 3.0-6.8. In further embodiments, the pH of the compositions or the products or the therapeutic medicines when dissolved is between 3.5-6.0, preferably 4.0-5.5, most preferably 4.5-5.0. In further embodiments, the ratios of N-acetylneuraminic acid methyl ester to N-acetylneuraminic acid of the compositions or the products or the therapeutic medicines are 1-5:1, preferably 1.25:1, most preferably 2-4:1. In further embodiments, the ratios of N-acetylneuraminic acid methyl ester to N-acetylneuraminic acid to sodium citrate or sodium acetate of the compositions or the products or the therapeutic medicines are 1-5:1:0.25-1 (N-acetylneuraminic acid methyl ester:N-acetylneuraminic acid:sodium citrate or sodium acetate), preferably 1.25-4:1:0.25-1, most preferably 2-4:1:0.25-1.

In another aspect, provided herein are the compositions or the products or the therapeutic medicines comprising N-acetylneuraminic acid methyl ester and methionine. In certain embodiments, the compositions or the products or the therapeutic medicines comprise N-acetylneuraminic acid and methionine. In certain embodiments, the compositions or the products or the therapeutic medicines comprise N-acetylneuraminic acid, N-acetylneuraminic acid methyl ester and methionine. In certain embodiments, the compositions or the products or the therapeutic medicines comprise N-acetylneuraminic acid, N-acetylneuraminic acid methyl ester, methionine and sodium citrate or sodium acetate. In certain embodiments, the pH of the compositions or the products or the therapeutic medicines when dissolved is between 3.0-6.8. In further embodiments, the pH of the compositions or the products or the therapeutic medicines when dissolved is between 3.5-6.0, preferably 4.0-5.5, most preferably 4.5-5.0.

In further embodiments, the ratios of N-acetylneuraminic acid methyl ester or N-acetylneuraminic acid to methionine are 0.2-1:1, preferably 1:1. In further embodiments, the ratios of N-acetylneuraminic acid methyl ester to N-acetylneuraminic acid to methionine are 0.2-1:0.2-1:1-2 (N-acetylneuraminic acid methyl ester:N-acetylneuraminic acid:methionine), preferably 1:1:2. In further embodiments, the ratios of N-acetylneuraminic acid methyl ester to N-acetylneuraminic acid to methionine to sodium citrate or sodium acetate of the compositions or the products or the therapeutic medicines are 0.2-1:0.2-1:1-2:0.25-1 (N-acetylneuraminic acid methyl ester:N-acetylneuraminic acid:methionine:sodium citrate or sodium acetate), preferably 1:1:2:1.

In further aspects, the compositions or the products are in a form of a tablet, a capsule (each including timed release and sustained release formulations), a pill, a powder mixture, a granule, an elixir, a tincture, a solution, a suspension, a syrup or a emulsion, a nasal drop or spray, an injectable, an infusion, a rehydration solution (oral or intravenous) or a rehydration salt, or a form conjugated to a nano-particle, or other using forms well known to those of ordinary skill in the relevant arts.

### IV. Dualistic Roles of Antibodies and the Possible Mechanisms of Action

Based on the traditional concept, the antibodies induced by an infectious pathogen or by a vaccine are protective to a host because they can neutralize the pathogen and prevent or treat the infectious disease. However, the roles of such antibodies can be dualistic. Without wishing to be bound to theory, it is thought that some of the antibodies can cross react to certain cells, tissues or organs of a host, triggers self-attack immune reactions such as antibody-dependent cytotoxicity (ADCC), or complement dependent cytotoxicity (CDC), or defects in signal transduction pathways, and cause damages or disorders of the tissues and organs. For example, anti-viral antibodies can bind to host tissues and organs, irritate and cause disorders of the tissues and organs (e.g. autoimmune diseases as described in PCT/US2009/039810 and PCT/US2014/25918).

Further, compared to control mouse pups, administration of high dose of the anti-rotavirus antibodies to mouse pups before or after rotavirus infection caused deaths or severer infection of mouse pups as described in PCT/US2009/039810. In a mouse model of influenza infection, administration of high dose of the anti-20009H1N1 (swine) antibodies before viral infection caused deaths or severer infection of mice compared to control mice infected with virus alone, as described in PCT/US2014/25918.

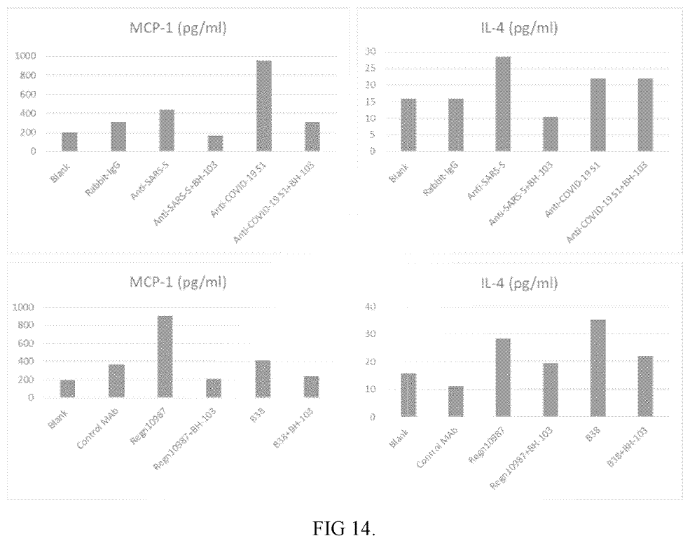

Sialic acids are predominant components at the out surface of cell membranes and mainly act as biological masks or receptors (Roland Schauer & Johannis P. Kamerling. Exploration of the sialic acid world. Elsevier, 2018, 12.1). Cells or tissues with sialic acid are recognized as “self”. After loss of sialic acid the cellular structures become “non-self” (R. Schauer & J. P. Kamerling. 2018) which can activate immune responses. During an infection of a virus using sialic acid as an attachment molecule, the sialic acid on the infected cells (e.g. lung epithelium cells) could be removed or destroyed by viruses carrying sialidase (e.g. influenza viruses) or receptor destroy enzyme (RDE, e.g. coronavirus). The current invention further discloses that certain antibodies against the spike protein of SARS-CoV-2 virus and SARS virus could significantly bind to the damaged lung epithelium cells and kidney embryonic cells with missed sialic acid on the cell surface, as shown in the Exemplification and FIG. 14.

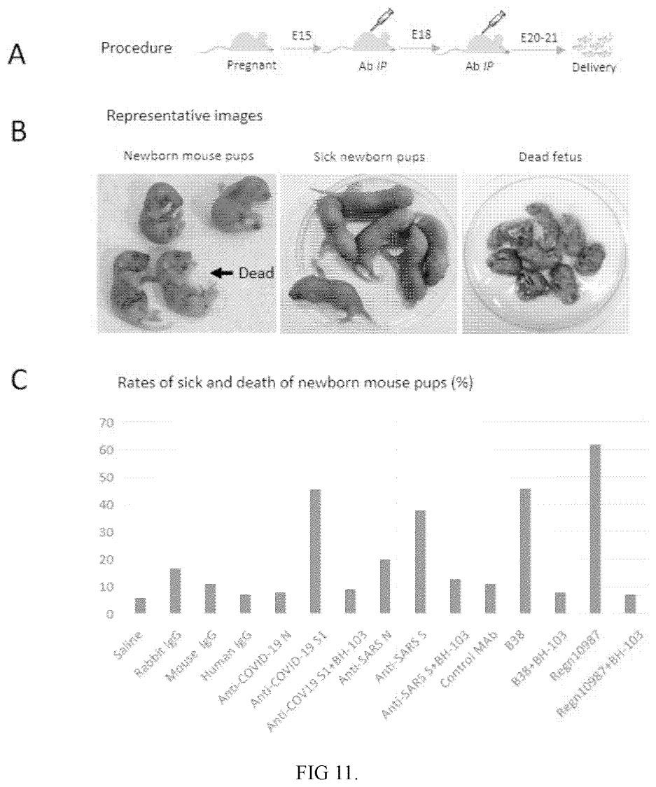

The antibody binding could mislead the immune response to attack self and induce the damage of multiple systems. For example, injection of high dose of the anti-rotavirus antibodies to pregnant mice induced deaths and bile duct epithelium proliferation (inflammation) of mouse pups born to the dames (PCT/US2009/039810); injection of human anti-influenza viral sera to pregnant mice induced fetal and neonatal deaths of mouse pups born to the dames (PCT/US2014/25918). Injection of the antibodies against the spike protein of SARS or SARS-CoV-2 virus (which causes the COVID-19 infection) to pregnant mice induced fetal and neonatal deaths of mouse pups born to the dames as described in Exemplification, FIGS. 11-13 and Table 1.

The in vitro and in vivo data support a new mechanisms of pathogenesis (MOP) of an highly pathogenic respiratory viral infection. The MOP include: 1) an highly pathogenic respiratory viruse such as the SARS-CoV-2 virus or the avian influenza virus causes the initial, primary damage (e.g. local inflammation and cellular damage) of its target organ (e.g. lung); 2) certain antibodies (e.g. anti-SARS-CoV-2 spike antibody) induced by the virus can bind to the damaged and the inflammatory cells of the target organ and other organs (e.g. heart, brain and kidney), mislead the immune response to attack the self cells or tissues and induces further damage (secondary damage); 3) the secondary damage can persistently add further damage to the primary damage and cause serious conditions (e.g. ARDS) even death as the antibodies elevate and reach the peak levels from week two to week three or four. 4) the overreacting immune responses (e.g. cytokine storm) misleaded by the pathogenic antibodies can be persistent and accumilated after viral clearance whenever the antibody exist.

The primary damage can be limited, short and decreased as the virus being cleared (such as a regular influenza infection). That means the virus itself is not enough to cause a serious condition such as ARDS or death. However, the secondary damage caused by the pathogenic antibodies can be longer and broader because antibodies persist much longer than viruses and can bind nonspecifically to other inflammatory tissues besides lung. The new MOP can explain why most patients with serious respiratory viral infections such as COVID-19 or avian influenza infection died after one week especially at 2-4 weeks, matching the period of antibody peak levels.

The new MOP of an highly pathogenic viral infection can also explain the serious adverse reactions observed with the vaccines of respiratory viruses such as the COVID-19 vaccines and the influenza vaccines. Similarly, certain pathogenic antibodies inducible by other infectious pathogens or other vaccines may also cause serious adverse reactions or autoimmune diseases through the similar pathogenic mechanism, even cancers if the inflammatory cellular proliferation stimulated by pathogenic antibodies loses control (e.g. cancers with HIV infected patients).

It should be noted that the majority (70% or more) of the anti-viral antibodies induced by either a virus or a vaccine is protective since the pathogenic antibodies take less than 30% according to the inventor's study.

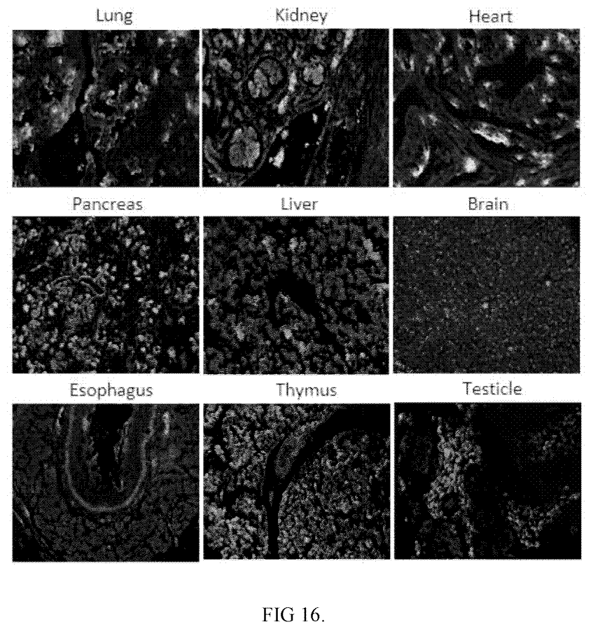

In the present disclosure, the term “pathogenic or dualistic antibodies” refers to any antibodies capable of causing pathogenic reactions and damages or disorders of the cells, tissues and organs of a host. The pathogenic antibodies can be induced during an infection (e.g. an influenza infection or a coronavirus infection) or a vaccination (e.g. an influenza or a coronavirus vaccination), or passively introduced (e.g. a therapeutic antibody). The diseases or conditions caused by pathogenic antibodies of the present disclosure include but not limited to infectious diseases, infection-relating diseases, complications and sequela of infections, COVID-19 long haulers, cytokine storm and cytokine release syndrome (CRS), adverse reactions of vaccines or therapeutic antibodies, inflammation, inflammatory respiratory diseases, inflammatory gastrointestinal diseases, infection-relating autoimmune diseases, allergy and infection-relating cancers, and any other disorders (known or unknown) inducible by pathogenic antibodies. In addition, pathogenic antibodies can bind to the unmatured fetal cells or tissues (FIG. 16) and cause abortions, postpartum labors, still births of pregnant females, and neonatal deaths and neonatal sudden deaths, as shown in the Exemplification and FIGS. 11-14.

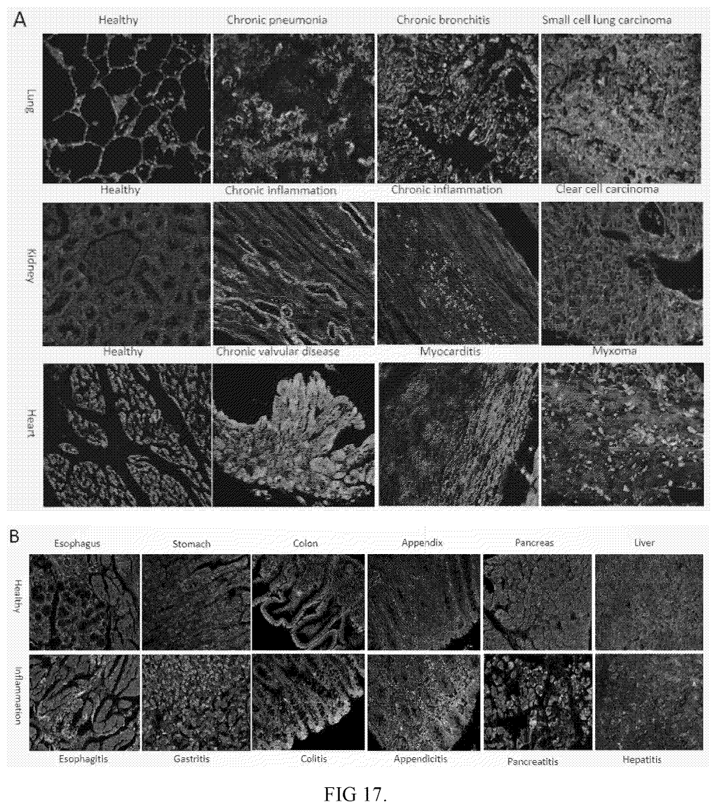

Cells or tissues vulnerable to pathogenic antibodies include but not limited to damaged cells with missing sialic acid, inflammatory cells or tissues, actively proliferating cells and tumor cells, etc. During an infection, pathogenic antibodies induced by a pathogen can bind to the vulnerable cells or tissues and rapidly activate immune responses to attack the antibody-bound cells or tissues. This MOP can explain why patients with chronic inflammatory diseases are more vulnerable to a highly pathogenic infection. Binding of an anti-SARS-CoV-2 spike antibody to human fetal tissues and various human disease tissues are shown in the Exemplification, FIG. 16 and FIG. 17.

### The Possible Mechanisms of Action

Many microbes bind to mammalian tissues by recognizing specific saccharide ligands. Thus saccharides and saccharide mimetics can be used to block the initial attachment of microbes to cell surface or block their release thus prevent and/or suppress infection (anti-adhesive). Because one of these organisms (e.g. rotavirus) naturally gains access through the gut, the saccharide-based drugs can be delivered directly without the requirement of being distributed systemically. Examples of such applications currently under study include milk oligosaccharides that are believed to be natural antagonists of gastrointestinal infection in infants; and polymers that will block the binding of viruses (Ajit Varli et al., Essentials of Glycobiology, Third Edition. Cold Spring Harbor Laboratory Press, 2017).

Certain sialic acids such as N-Acetylneuraminic acid as a component of viral receptors are related to viral attachment of influenza viruses or coronavirus. (James Stevens et al. 2006. Science, Vol 312; Xinchuan Huang, et al. 2015. J Virology, Vol 89). Sialic acid has a negative charge on its surface, it is not easy to enter cells and participate the sugar chain synthesis process, and is metabolized rapidly in vivo. Therefore it is difficult for sialic acid alone to be used as a clinic therapeutic. An analog or derivative of N-Acetylneuraminic acid, N-Acetylneuraminic acid methyl ester is easier to enter cells compared with N-Acetylneuraminic acid.

In addition, chemical modification of sialic acids can strongly influence all of their properties, in particular ligand functions. The hydroxyl groups present in both monosaccharides and oligosaccharides can be chemically modified without affecting the glycosidic linkages. Methylation is used in the structural analysis of glycans. Natural products containing partially methylated glycans are known and a number of methyltransferases have been identified (Ajit Varli et al., Essentials of Glycobiology, Third Edition. Cold Spring Harbor Laboratory Press, 2017: Chapter 2). For example, O-methylation can hinder or even prevent hydrolysis of the glycosidic bond by sialidase. Other substitution of the hydroxyl groups of sialic acids arises from use of the appropriate donors. For example, S-adenosylmethionine for methylated sialic acid or 5′-phosphosulfate for sulfated molecules (Ajit Varli et al., Essentials of Glycobiology, Third Edition. Cold Spring Harbor Laboratory Press, 2017: Chapter 15).

One embodiment of the present invention is using methyl- and sulfur-containing molecules as donors for methylation and sulfidation of sialic acids or other saccharides. For example, N-acetylneuraminic acid methyl ester may act as a donor of methyl organ. Methionine contains —S—CH₃ thus may act as a donor sulfur and methyl to modify a pathogen binding site (a sialic acid or a saccharide) into methylated and sulfated forms. Such chemical modification of a sialic acid or a saccharide may attenuate even prevent pathogen binding and block the pathogen's entry into the host cells.

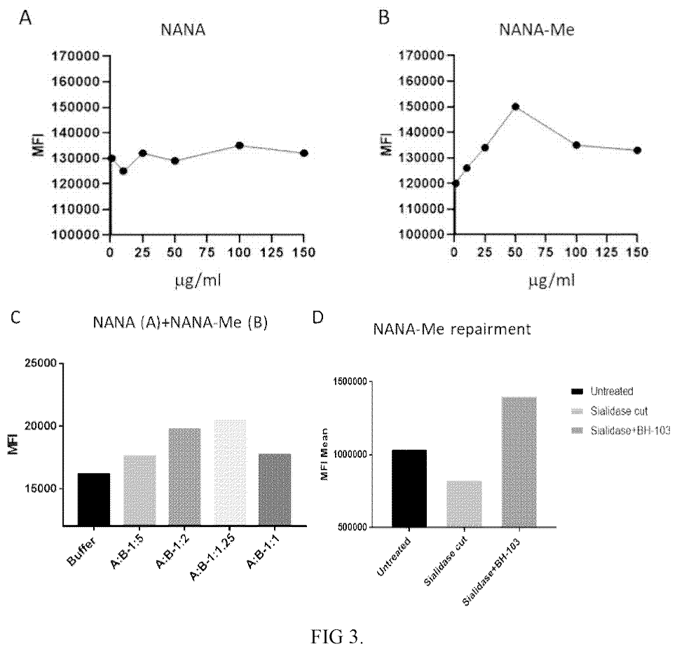

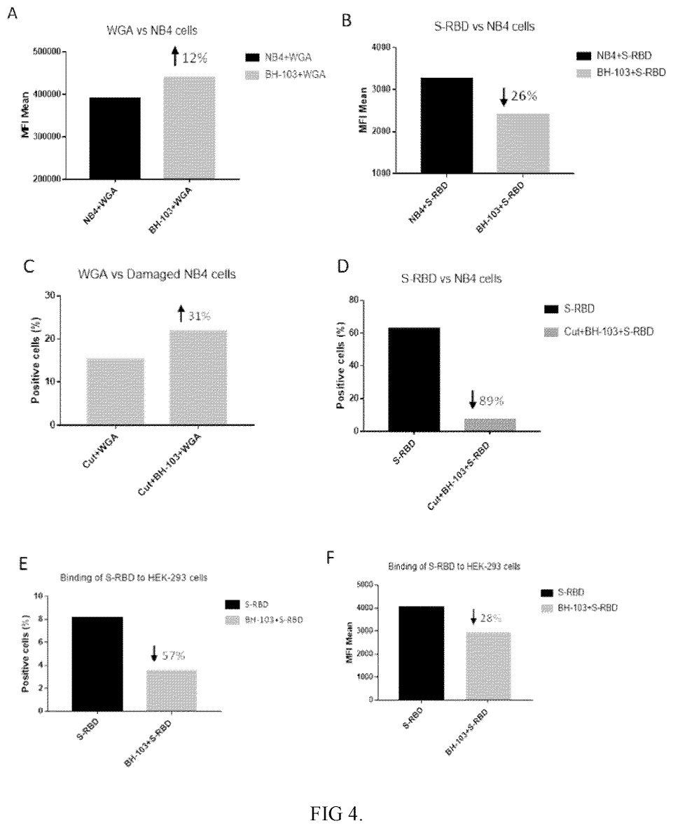

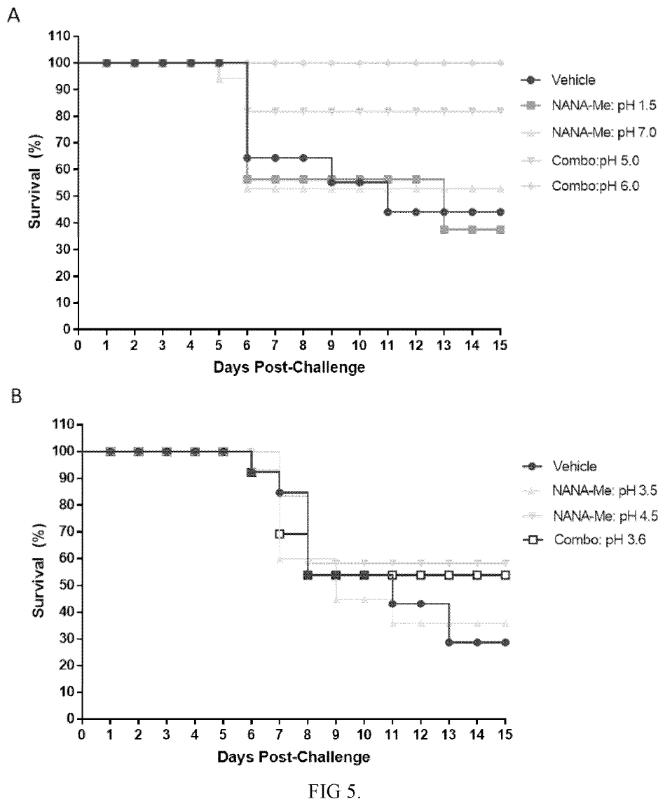

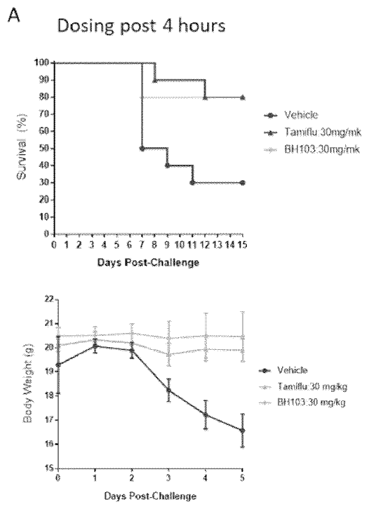

Therefore, one embodiment of the present invention is to disclose the unique functions of N-acetylneuraminic acid methyl ester. As shown in the Exemplification and FIG. 3D, N-acetylneuraminic acid methyl ester is helpful for the repairment of missing sialic acid on the damaged lung epithelium cells. The structure recovery of the damaged cells can reduce the self-attack of immune system to the damaged or inflammatory cells and prevent the damage of lung and other organs (Mechanism of Action, MOA-1). Moreover, the replacement of N-acetylneuraminic acid by N-acetylneuraminic acid methyl ester could induce a structural or chemical modification of the viral receptor that significantly decreased the binding affinity of the SARS-CoV-2 viral receptor binding domain (MOA-2) (FIG. 4).

Based on the MOA-2, N-acetylneuraminic acid methyl ester can prevent the COVID-19 infection by blocking viral entry into host cells, and treat the infection by blocking viral entry into new cells. More importantly, the structure repairment of the damaged cells by N-acetylneuraminic acid methyl ester can reduce the self-attack of immune responses and prevent the systemic damage of lung and other organs (MOA-1). Because sialic acid is a receptor for not only coronavirus but also other viruses (e.g. influenza viruses or rotavirus) the receptor modification and blocking entry, and the structural repairment by N-acetylneuraminic acid methyl ester should be widely effective for the prevention and treatment of other infections of other viruses using sialic acids as receptors (e.g. influenza viruses or rotavirus).

As a support, the data of in vivo study of influenza viral infections indicated that compositions comprising N-acetylneuraminic acid methyl ester or N-acetylneuraminic acid methyl ester and N-acetylneuraminic acid, could effectively inhibit an over-reacting immune response by significantly reducing the damage of lung and other organs, decrease the death rate, and reducing cytokine release, as shown in the Exemplification, FIGS. 5-9. Thus N-acetylneuraminic acid methyl ester is effective for the treatment and prevention of severe patients with respiratory viral infections.

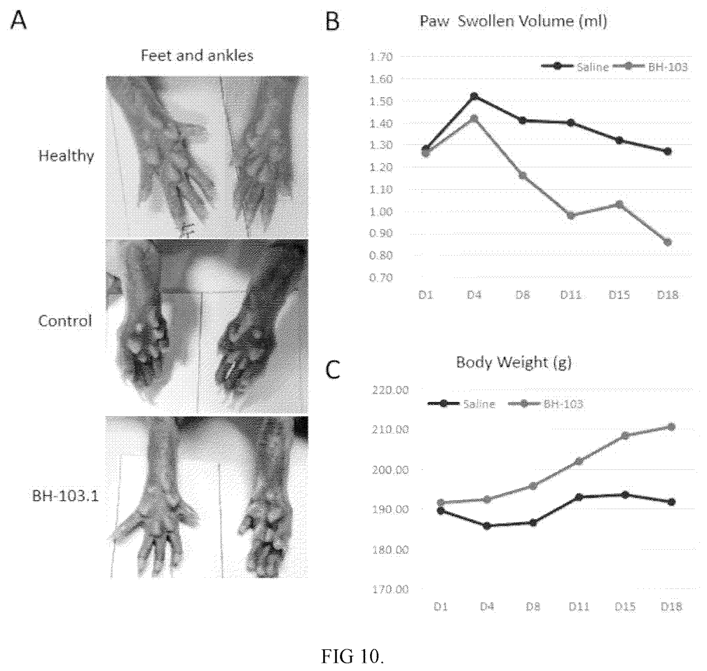

As another support, the data of in vivo study also indicated that the compositions or the products comprising N-acetylneuraminic acid methyl ester and N-acetylneuraminic acid are effective for the prevention and treatment of rheumatoid arthritis, as shown in the Exemplification and FIG. 10; and can be widely effective for the treatment of other autoimmune diseases particularly those caused by pathogenic antibodies as described in any of the preceding embodiments.

As further support, the pathogenic antibodies can bind to the unmatured fetal cells or tissues (FIG. 16) and cause abortions, postpartum labors, still births of pregnant females, and neonatal deaths and neonatal sudden deaths, as shown in the Exemplification and FIGS. 11-14. As further support, the pethogenic antibodies can bind to the human inflammatory tissues or cancer tissues of respiratory, cardiovesvular, urinary, and digestive system (FIG. 17), and cause serious infections particularly highly pathogenic viral infections (e.g. COVID-19 infection), serious adverse reactions of vaccines (e.g. COVID-19 vaccines), serious complications of infections (e.g. ARDS or cytokine storm), infection-relating inflammation and autoimmune diseases including COVID-19 long haulers, and infection-relating cancers which occur if an inflammatory cellular proliferation stimulated by an pathogenic antibody repeatedly persists for long time and loses control.

Thus the compositions or the products comprising N-acetylneuraminic acid methyl ester of the current disclosure are widely effective for the treatment and prevention of the diseases or conditions caused by pathogenic antibodies as described in any of the preceding embodiments. The pathogenic antibodies can be induced by a pathogen particularly a highly pathogenic virus (e.g. the SARS-CoV-2 virus), a vaccine particularly a vaccine of a highly pathogenic virus (e.g. a COVID-19 vaccine), or a therapeutic antibody.

Numerous other objects, features and advantages of the present disclosure will become readily apparent from the detailed description. It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### V. Saccharide Related Diseases

Certain aspects of the present disclosure are related to saccharide related diseases particularly the diseases or conditions caused by missed sialic acid on a cell surface and pathogenic antibodies.

As used herein, the term “saccharide related disease” refers to the diseases or conditions caused by abnormal glycosylation or pathogenic antibodies. Specific cell surface proteins are modified by the glycosylation of saccharides. The patterns of glycosylation change can affect a physiological or a pathological process such as molecular recognition and adhesion, signal transduction and activation or inhibition of immune system. The modification of the physiological or a pathological process can cause diseases or abnormal conditions. Nearly all types of malignant cells and many types of diseased tissues demonstrate alterations in their glycosylation patterns (Blomme et al., 2009; Danussi et al., 2009; Patsos et al., 2009). Certain saccharide such as sialic acids as components of viral receptors are related to viral attachment of influenza viruses or coronavirus (James Stevens et al. 2006. Science, Vol 312; Xinchuan Huang, et al. 2015. J Virology, Vol 89).

Sialic acids are predominant components at the out surface of cell membranes and mainly act as biological masks or receptors (Roland Schauer & Johannis P. Kamerling. Exploration of the sialic acid world. Elsevier, 2018, 12.1). Cells or tissues with sialic acid are recognized as “self”. After loss of sialic acid the cellular structures become “non-self” (R. Schauer & J. P. Kamerling. 2018) which can activate immune responses. Damaged cells or tissues with missing sialic acid, or inflammatory cells or tissues, or actively proliferating cells and tumor cells are vulnerable to pathogenic antibodies as mentioned above. During an infection, pathogenic antibodies induced by a pathogen can bind to the vulnerable cells or tissues and rapidly activate immune responses to attack the antibody-bound cells or tissues, and cause systemic injury of multiple organs. This MOP can cause serious infections, serious complications and sequela of infections, long haulers of an infection (e.g. COVID-19 long haulers), systematic inflammation and injury of multiple organs, adverse reactions of vaccines or therapeutic antibodies, infection-relating autoimmune diseases, allergies and cancers. This MOP can also cause abortion, postpartum labor, still birth of pregnant females, neonatal death and neonatal sudden death, caused by an infection or by a vaccine.

Thus the saccharide related diseases, preferably the diseases or conditions caused by missing sialic acid on a cell surface and pathogenic antibodies in the present disclosure, include but not limited to infectious diseases, infection-relating diseases, complications and sequela of infections, COVID-19 long haulers, cytokine storm and cytokine release syndrome (CRS), adverse reactions of vaccines or therapeutic antibodies, inflammation, inflammatory respiratory diseases, inflammatory gastrointestinal diseases, autoimmune diseases, allergy and cancers. The saccharide related diseases also include but not limited to abortion, postpartum labor, still birth of pregnant females, neonatal death and neonatal sudden death, caused by an infection or by a vaccine.

### Infectious Diseases

As used herein, the term “infectious diseases” refers to the invasion of a host organism's bodily tissues by disease-causing organisms, their multiplication, and the reaction of host tissues to these organisms and the toxins they produce. A short-term infection is an acute infection. A long-term infection is a chronic infection. Pathogens specific to infectious diseases suitable for use in this process include but not limited to viruses, bacteria, parasites, fungi, viroids, prions, protozoa, and insects, and etc., without limitation. Examples of infections include but not limited to the disorders caused by influenza viruses, coronaviruses, avian infectious bronchitis virus (IBV), avian coronavirus, reoviruses, rotaviruses, cytomegaloviruses (CMV), Epstein-Barr viruses (EBV), adenoviruses, hepatitis viruses including HAV, HBV, HCV, human immunodeficiency virus (HIV), human T-cell leukemia viruses (HTLV), human papilloma viruses (HPV), polio viruses, parainfluenza viruses, measles viruses, mumps viruses, respiratory syncytial viruses (RSV), human herpes viruses (HHV), herpes simplex virus (HSV), Varicella-Zoster Virus, cholera viruses, pox virus, rabies virus, distemper virus, foot and mouth disease viruses, rhinoviruses, Newcastle disease viruses, pseudorabies virus, cholera, syphilis, anthrax, leprosy and bubonic plague, rickettsias, Neisseria gonorrhoeae, Bordetella pertussis, Escherichia coli, Salmonella enterica, Vibrio cholerae, Pseudomonas aeruginosa, Yersinia pestis, Francisella tularensis, Haemophilus influenzae, purple sulfur bacteria, Helicobacter pylori, Campylobacter jejuni, Bacillus anthracis/cereus/thuringiensis, Clostridium tetani, Clostridium botulinum, staphylococci, streptococci, pneumococci, Streptococcus pneumoniae, mycoplasmas, Bacteroides fragilis, Mycobacterium tuberculosis, Mycobacterium leprae, Corynebacterium diphtheriae, Treponema pallidum, Borrelia burgdorferi, Chlamydia trachomatis, Chlamydia psittaci, phycocyanin, phycoerythrin, mitochondria, chloroplasts, etc without limitation.

As used herein, the term “infection-relating diseases” refers to the disorders or conditions occurred during or after an infection. According to the present invention, infection-relating diseases or conditions include but not limited to the complications or sequela of infections, autoimmune diseases, allergies, inflammation and tumors occurred during or after an infection. The disorders or conditions usually arise after a period time (e.g. within 2-6 weeks) of an infection. Examples of infection-relating autoimmune diseases, allergies, inflammation and tumors include but not limited to cytokine storm, cytokine release syndrome, Guillain-Barre syndrome, autism, Kawasaki's disease, biliary atresia, primary biliary cirrhosis, systemic lupus erythematous, leukemia, acute leukemia, rheumatoid arthritis, adult onset diabetes mellitus (Type II diabetes), Sjogren's syndrome, juvenile onset diabetes mellitus, Hodgkin's and non-Hodgkin's lymphoma, malignant melanoma, cryoglobulinemia, inflammatory bowel disease, polymyositis, dermatomyositis, multiple endocrine failure, Schmidt's syndrome, autoimmune uveitis, Addison's disease, adrenalitis, Graves' disease, thyroiditis, Hashimoto's thyroiditis, autoimmune thyroid disease, subacute cutaneous lupus erythematosus, hypoparathyroidism, autoimmune thrombocytopenia, autoimmune hemolytic anemia, dermatitis herpetiformis, autoimmune cystitis, male or female autoimmune infertility, ankylosing spondylitis, ulcerative colitis, Crohn's disease, mixed connective tissue disease, polyarteritis nodosa, systemic necrotizing vasculitis, juvenile onset rheumatoid arthritis, rheumatic fever, asthma, recurrent abortion, Behcet's disease, endocarditis, myocarditis, endomyocardial fibrosis, endophthalmitis, Alzheimer's disease, post vaccination syndromes, and any other disorder or conditions in which the specific recognition of the host by pathogen-inducible or vaccine-inducible antibodies is suspected or shown to be important in any aspect of the pathogenesis of the clinical illness. The infection-relating diseases further include but not limited to abortion, postpartum labor, still birth of the pregnant females, and neonatal death and neonatal sudden death, caused by an infection or by a vaccine.

### Complications or Sequela of Infections

The complication of an infection refers to the disorders or conditions occurred during the infection. The sequela of an infection refers to the disorders or conditions occurred after the infection. The complications or the sequela of the COVID-19 infection or an highly pathogenic influenza infection or other infections include but not limited to acute respiratory failure, pneumonia, acute respiratory distress syndrome (ARDS), acute kidney injury, acute cardiac injury, acute liver injury, acute injury of neural system, Bell's palsy, secondary infection, septic shock, blood clots, disseminated intravascular coagulation, multisystem inflammatory syndrome in children, chronic fatigue, fibrotic lung, new-onset diabetes, stroke, heart attack, new-onset epilepsy, psychological illness, easy clotting/thrombosis, high fever, swelling and redness, extreme fatigue, nausea, Acute Disseminated Encephalomyelitis (ADEM), Guillian Barre Syndrome (GBS), meningitis, encephalitis, rhabdomyolysis, cytokine storms, cytokine release syndrome, bacteremia, sepsis, bronchitis, sinutis, enlarged tonsils, tonsillitis, swollen lymph nodes (bull neck), myocarditis, infectious mononucleosis, heart attacks, strokes, high fever, swelling and redness, extreme fatigue, nausea, cytokine storms, autoimmune diseases, deaths, etc.

### COVID-19 Long Hauler

COVID-19 symptoms can last weeks or months for some people. These patients, given the name “long haulers”, have in theory recovered from the worst impacts of COVID-19 and have tested negative. However, they still have symptoms. The most common long hauler symptoms include but not limited to coughing, ongoing, sometimes debilitating, fatigue, body aches, joint pain, shortness of breath, loss of taste and smell, difficulty sleeping, headaches, brain fog, etc. Brain fog refers to unusually forgetful, confused or unable to concentrate even enough to watch TV.

### Adverse Reactions of Vaccines or Therapeutic Antibodies

As used herein, the term “adverse reactions” of vaccines or therapeutic antibodies of the present disclosure refers to the severe disorders or conditions caused by pathogenic antibodies induced during a vaccination or an antibody therapy. The vaccines include but not limited to the vaccines of influenza viruses, coronaviruses including SARS, SARS-CoV-2, IBV, MERS, and all the pathogens as mentioned above. The therapeutic antibodies include but not limited to any known or unknown antibody product which cause serious adverse reactions during a clinical intervention. The disorders or conditions usually arise after a period time (e.g. within 2-6 weeks) of a vaccination or an antibody therapy. Examples of serious adverse reactions of vaccines or therapeutic antibodies of the present disclosure include but not limited to deaths, coagulation abnormality, thrombocytopenia, stroke, blood clots, disseminated intravascular coagulation, Bell's palsy, acute infant death syndrome, cytokine storm, cytokine release syndrome, Guillain-Barre syndrome, Kawasaki's disease, acute leukemia, allergies, serious allergic reactions, asthma, epilepsy, immune system disorders, behavior disorders, nervous system disorders or injury, permanent brain damage, learning difficulties, seizure, severe seizures, lowered consciousness, autism, long-term coma, headaches, upper or low respiratory tract infection, joint pain, abdominal pain, cough, nausea, diarrhea, high fever, blood in the urine or stool, pneumonia, inflammation of the stomach or intestines, non-stop crying, fainting, deafness, temporary low platelet count, hives, pain in the joints, intussusception, vomiting, severe nervous system reaction, life-threatening severe illness with organ failure, still birth, neonatal deaths, and any other disorder or conditions in which an infection of the host is suspected or shown to be important in any aspect of the pathogenesis of the clinical illness, blood clots, disseminated intravascular coagulation, heart attacks, etc.

### Inflammation

As used herein, the term “inflammation” refers to the complex biological response of body tissues to harmful stimuli, such as pathogens, damaged cells, or irritants. The classical signs of acute inflammation include without limitation pain, heat, redness, swelling, and loss of function. Inflammation is a generic response, and therefore it is considered a mechanism of innate immunity. Inflammation can be classified as acute or chronic. Acute inflammation refers to the initial response of the body to harmful stimuli and is achieved by the increased movement of plasma and leukocytes (especially granulocytes) from the blood into the injured tissues. A series of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation and is characterized by simultaneous destruction and healing of the tissue from the inflammatory process. Inflammatory cells usually have abnormal glycosylation such as missing sialic acid on the cellular surface and are vulnerable to the pathogenic antibodies.

### Cytokine Storm or Cytokine Release Syndrome

Cytokines are a group of proteins. Through a process called cell signaling—communication between cells—cytokines control inflammation. During an infection, immune system releases more cytokines. Cytokine release syndrome (CRS) is a form of systemic inflammatory response syndrome (SIRS) that can be triggered by a variety of factors such as infections and certain drugs. It occurs when large numbers of white blood cells are activated and release inflammatory cytokines, which in turn activate yet more white blood cells. CRS is also an adverse effect of some monoclonal antibody medications, as well as adoptive T-cell therapies. When occurring as a result of a medication, it is also known as an infusion reaction.

As the body loses control of cytokine production, it results in a “cytokine storm”. The term cytokine storm is often used interchangeably with CRS but, despite the fact that they have similar clinical phenotype, their characteristics are different. While CRS can be an immunotherapy side effect, cytokine storms are related to infections. This can occur when the immune system is fighting pathogens, as cytokines produced by immune cells recruit more effector immune cells such as T-cells and inflammatory monocytes (which differentiate into macrophages) to the site of inflammation or infection. In addition, pro-inflammatory cytokines binding their cognate receptor on immune cells results in activation and stimulation of further cytokine production. This process, when dysregulated, can be life-threatening due to systemic hyper-inflammation, hypotensive shock, and multi-organ failure. Symptoms of cytokine storms or CRS include fever, fatigue, loss of appetite, muscle and joint pain, nausea, vomiting, diarrhea, rashes, fast breathing, rapid heartbeat, low blood pressure, seizures, headache, confusion, delirium, hallucinations, tremor, and loss of coordination, etc.

### Inflammatory Respiratory Diseases

As used herein, the term “respiratory tract” refers to the structures of the anatomy involved with the process of respiration. The respiratory tract is divided into 3 segments: upper respiratory tract including nose and nasal passages, paranasal sinuses, and throat or pharynx; respiratory airways including voice box or larynx, trachea, bronchi, and bronchioles; and lungs including respiratory bronchioles, alveolar ducts, alveolar sacs, and alveoli. Most of the respiratory tract exists merely as a piping system for air to travel in the lungs, and alveoli are the only part of the lung that exchanges oxygen and carbon dioxide with the blood. The respiratory tract is a common site for infections. Upper respiratory tract infections are probably the most common infections in the world.

“Respiratory diseases” refers to an abnormal status or conditions of the upper respiratory tract, trachea, bronchi, bronchioles, alveoli, pleura and pleural cavity, and the nerves and muscles of breathing. Respiratory diseases range from mild and self-limiting. Respiratory diseases can be classified as many types. Inflammatory lung diseases include but not limited to viral pneumonia, asthma, cystic fibrosis, emphysema, chronic obstructive pulmonary disorder, acute respiratory distress syndrome (ARDS), or acute respiratory diseases (ARD). Obstructive lung diseases include but not limited to chronic obstructive pulmonary disease (COPD), which includes emphysema and asthma. Asthma is a difficulty in breathing causing wheezing due to inflammation of bronchi and bronchioles, this causes a restriction in the airflow into the alveoli. Respiratory tract infections can affect any part of the respiratory system. The upper respiratory tract infection include but not limited to common cold, sinusitis, tonsillitis, otitis media, pharyngitis and laryngitis. The lower respiratory tract infection includes but not limited to pneumonia, a lung infection. Pneumonia is usually caused by bacteria, particularly Streptococcus pneumoniae in Western countries. Worldwide, tuberculosis is an important cause of pneumonia. Other pathogens such as viruses and fungi can cause pneumonia for example severe acute respiratory syndrome. A pneumonia may develop complications such as a lung abscess, a round cavity in the lung caused by the infection, or may spread to the pleural cavity, and the damages of other organs. Other examples of respiratory diseases or conditions include but not limited to influenza infections, coronavirus infections, common cold, entities like viral or bacterial pneumonia, etc.

### Inflammatory Gastrointestinal Diseases

Gastrointestinal diseases refer to an abnormal status or function of the esophagus, stomach and intestine. Examples of inflammatory gastrointestinal diseases or conditions include but not limited to diarrhea, gastroenteritis, ileitis, colitis, coeliac disease, inflammatory bowel disease (IBD), Crohn's disease and ulcerative colitis, irritable bowel syndrome (IBS), chronic functional abdominal pain, pseudomembranous colitis, esophagitis, gastritis, etc.

### Diarrhea

Diarrhea is defined by the WHO as having three or more loose or liquid stools per day, or as having more stools than is normal for that person. The same definition is also suitable for animals.

Diarrhea may be caused by an infection or a chronic gastrointestinal disease. The common causes of diarrhea include but not limited to: 1) bacterial infections such as infections caused by Clostridium, Campylobacter, Salmonella, Shigella, Giardia and Escherichia coli; 2) Viral infections such as infections caused by rotavirus, coronavirus, Norwalk virus, cytomegalovirus, herpes simplex virus and viral hepatitis; 3) nutritional problems or food intolerances. Some people are unable to digest certain component of food such as lactose; and nutritional diarrhea is most common in orphaned animals as a result of dietary changes, poor quality milk replacers, mixing errors, and overfeeding. 4) parasites such as Giardia lamblia, Entamoeba histolytica, and Cryptosporidium; 5) reactions to medicines such as antibiotics, blood pressure medications and antacids containing magnesium; 6) inflammatory gastrointestinal diseases such as inflammatory bowel disease (IBD) or celiac disease, tuberculosis, colon cancer, and enteritis; 7) functional bowel disorders such as irritable bowel syndrome.

### Inflammatory Bowel Disease (IBD)

As is known in the art, many gastrointestinal diseases such as inflammatory bowel disease (IBD) may present symptoms in tissues including without limitation the small and large intestines, mouth, stomach, esophagus, and anus. An IBD of the present disclosure may be chronic or acute. The term “inflammatory bowel disease (IBD)” refers to the pathological state characterized by chronic or acute inflammation of all or part of digestive tract. IBD primarily includes ulcerative colitis and Crohn's disease. Both usually involve severe diarrhea, pain, fatigue, and weight loss. Ulcerative colitis is a form of IBD that causes long-lasting inflammation and sores (ulcers) in large intestine (colon) and rectum. Crohn's disease is a form of IBD that causes inflammation of the digestive tract. In Crohn's disease, inflammation often spreads deep into affected tissues. The inflammation can involve different areas of the digestive tract such as the large intestine, small intestine or both. Collagenous colitis and lymphocytic colitis also are considered inflammatory bowel diseases but are usually regarded separately from classic inflammatory bowel disease. In some embodiments, an IBD may include colitis (such as diversion, lymphocytic, collagenous, or indeterminate colitis) or Behcet's disease.

### Arthritis

Arthritis is the swelling and tenderness of one or more joints. The main symptoms of arthritis are joint pain and stiffness, which typically worsen with age. The most common types of arthritis are osteoarthritis and rheumatoid arthritis. Osteoarthritis causes cartilage—the hard, slippery tissue that covers the ends of bones where they form a joint—to break down. Rheumatoid arthritis is a disease in which the immune system attacks the joints, beginning with the lining of joints. Other types of arthritis include but not limited to ankylosing spondylitis, gout, juvenile idiopathic arthritis, osteoarthritis, psoriatic arthritis, reactive arthritis, septic arthritis, thumb arthritis, etc.

### VI. Methods of Treatment

In certain aspect that may be combined with any of the preceding embodiments, the present invention discloses the methods for the treatment and/or prevention of saccharide related diseases, comprising administering an effective amount of at least one of the compositions or the products consisted of suitable amounts of an analog of a sialic acid (e.g. N-acetylneuraminic acid methyl ester) alone or an analog of a sialic acid plus at least one of the other major components (e.g. N-acetylneuraminic acid or methionine) of any of the preceding embodiments of the present disclosure, to a patient or an individual suffering or developing a saccharide related disease of any of the preceding embodiments. In certain embodiments, the pH of the compositions or the products when dissolved is between 3.0-6.8, preferably 3.5-6.0, preferably 4.0-5.5, most preferably 4.5-5.0.

In some embodiments, the compositions or the products of any of the preceding embodiments are used for the prevention and treatment of saccharide related diseases of any of the preceding embodiments as: 1) therapeutic products; 2) food or feed additives or products comprising the additives; 3) dietary supplement products to help supporting or enhancing the protective structure or function of cells or tissues in particular respiratory or/and gastrointestinal tracts of humans and animals; and 4) oral rehydration salt or oral rehydration solution (ORS) for the prevention and treatment of dehydration due to diarrhea or fever of humans and animals.

In some embodiments, the effective dosages of the compositions or products of any of the preceding embodiments for the uses are from about 0.01 mg/kg to about 200 mg/kg of an analog or derivative of a sialic acid (e.g. N-acetylneuraminic acid methyl ester) or a sialic acid (e.g. N-acetylneuraminic acid), from about 0.005 mg/kg to about 100 mg/kg of an additive salt (e.g. sodium citrate or sodium acetate).

In some embodiments that may be combined with any of the preceding embodiments, the administrating routes of a therapeutic product include but not limited to subcutaneous, topical with or without occlusion, oral, intramuscular, intravenous (both bolus and infusion), intraperitoneal, intracavity, or transdermal, inhalant, or other using forms well known to those of ordinary skill in the pharmaceutical arts.

In some embodiments that may be combined with any of the preceding embodiments, the saccharide related diseases include but not limited to at least one of infectious diseases, infection-relating diseases, complications and sequela of infections, COVID-19 long haulers, cytokine storm and cytokine release syndrome (CRS), adverse reactions of vaccines or therapeutic antibodies, inflammation, inflammatory respiratory diseases, inflammatory gastrointestinal diseases, autoimmune diseases, allergy and cancers. In further embodiments that may be combined with any of the preceding embodiments, the saccharide related diseases include but not limited to abortion, postpartum labor, still birth of pregnant females, neonatal death and neonatal sudden death, caused by an infection or by a vaccine.

In some embodiments, a patient or an individual include but not limited to a human or an animal. In some embodiments, the humans include but not limited to males and females, newborns, 1-12 months old infants, 1-18 years old children, adults, old people, pregnant and feeding females, or the pregnant or feeding females with their fetus or suckling babies at risk of developing saccharide related diseases. In some embodiments, the animals include but not limited to livestock animals including but not limited to cows, pigs, horses, sheep or goats, llamas, cattle, donkeys; poultry including but not limited to chickens, ducks, gooses, turkeys and pigeons; companion animals including but not limited to dogs, cats, rodent pets and avian pets. More specifically, the livestock animals include but not limited to males and females, adult animals, newborn animals, infant animals, and other young age animals, pregnant and feeding female animals.

As described below, these therapeutic products or compositions are effective in preventing and treating different types of the saccharide related diseases in various in vitro and in vivo models. In addition, these therapeutic products or compositions were found to be better compared to previous. One example of the compositions or products containing at least two of the major components of the present disclosure comprise suitable amounts of an analog of the sialic acid (e.g. N-acetylneuraminic acid methyl ester) and a sialic acid (e.g. N-acetylneuraminic acid); wherein the pH of the compositions or the products when dissolved is between 3.0-6.8, preferably 3.5-6.0, preferably 4.0-5.5, most preferably 4.5-5.0.

### Infectious Diseases or Respiratory Infections

As described herein, the methods of the present disclosure are effective against a wide range of infectious diseases or infection-relating diseases in an individual. In some embodiments that may be combined with any of the preceding embodiments, the individual has a viral infection. A viral infection of the present disclosure may be chronic or acute. In some embodiments that may be combined with any of the preceding embodiments, a viral infection may include respiratory viral infections such as influenza viral infection or coronavirus infection. In some embodiments that may be combined with any of the preceding embodiments, an influenza infection is caused by at least one of the H1N1, H3N2, H5N1, H7N9, H7N8, or H9N2 virus and any variants or newly emerging strains of the influenza viruses. In some embodiments that may be combined with any of the preceding embodiments, a coronavirus infection is caused by at least one of the SARS-CoV-2, SARS and MERS virus and any variants or newly emerging strains of the coronaviruses. The prevention and treatment of COVID-19 infections (through blocking viral entry) with a composition consisted of N-acetylneuraminic acid methyl ester and N-acetylneuraminic acid and sodium citrate in cellular models are described in the Exemplification and shown in FIG. 4. The prevention and treatment of H1N1 and H3N2 infections with a composition consisted of N-acetylneuraminic acid methyl ester alone or N-acetylneuraminic acid methyl ester and N-acetylneuraminic acid in mouse models are described in the Exemplification and shown in FIGS. 5-9.

### Complications and Sequela of Infectious Diseases

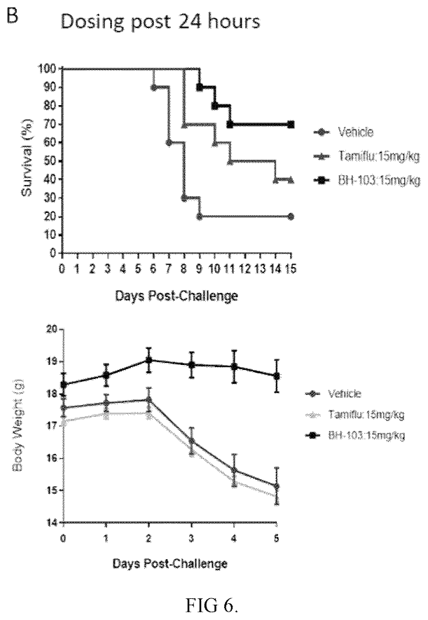

As described herein, the methods of the present disclosure are effective against a wide range of complications and sequela of infectious diseases or infection-relating diseases preferably caused by pathogenic antibodies in an individual. In some embodiments that may be combined with any of the preceding embodiments, the individual has ARDS or ARD. In some embodiments that may be combined with any of the preceding embodiments, the individual has cytokine storm or CRS. In some embodiments that may be combined with any of the preceding embodiments, the individual has a systematic inflammation or injury of kidney, cardiovascular, neural system, liver or/and digestive system. In some embodiments that may be combined with any of the preceding embodiments, the individual has COVID-19 long haulers. The prevention and treatment of the complications of H1N1 and H3N2 infections with a composition consisted of N-acetylneuraminic acid methyl ester and N-acetylneuraminic acid in in mouse models are described in the Exemplification and shown in FIGS. 6-9.

Mouse models are described in the Exemplification and shown in FIGS. 5-9. The complications of the H1N1 and the H3N2 infections included ARDS or ARD, cytokine storm or CRS, inflammation and systematic injury of kidney, cardiovascular, neural system, liver or/and digestive system, and those as described in the part of “Saccharide related diseases” of the application. Based on the MOP disclosed in the application, the complications and sequela of infectious diseases or COVID-19 long haulers are caused by some of the antibodies induced during an infection. Since the compositions or the products of the present disclosure are effective for the prevention and treatment of the symptoms caused by the pathogenic antibodies against SARS-CoV-2 spike proteins, the same compositions of the present disclosure can be effective for treating one or more symptoms of the sequela or long haulers of COVID-19 infection, and of other viral infections (e.g. influenza infections).

### Adverse Reactions of Vaccines or Therapeutic Antibodies

As described herein, the methods of the present disclosure are effective against a wide range of adverse reactions of vaccines or therapeutic antibodies, particularly those caused by pathogenic antibodies in an individual. Vaccines known to induce serious adverse reactions may include but not limited to vaccines of influenza viruses, coronavirus, and other viruses. In some embodiments, the influenza vaccines are made of at least one of the H1N1, H3N2, H5N1, H7N9, H7N8, or H9N2 virus and any variants or newly emerging strains of the influenza viruses, or the information from the viruses. In some embodiments, a coronavirus vaccine is made of at least one of the SARS-CoV-2, SARS, IBV, and MERS virus and any variants or newly emerging strains of the coronaviruses, or the information from the viruses. The prevention and treatment of the adverse reactions of the antibodies inducible by coronavirus vaccines such as COVID-19 vaccines with a composition consisted of N-acetylneuraminic acid methyl ester and N-acetylneuraminic acid and sodium citrate in a pregnant mouse model, are described in the Exemplification and shown in FIGS. 11-14. The adverse reactions caused by the antibodies inducible by coronavirus vaccines such as COVID-19 vaccines and SARS-CoV virus include deaths, coagulation abnormality, thrombocytopenia, stroke, blood clots, disseminated intravascular coagulation, Bell's palsy, acute infant death syndrome, cytokine storm, cytokine release syndrome, Guillain-Barre syndrome, inflammation and systematic injury of kidney, heart, neural system, liver or/and digestive system, abortion, postpartum labor, still birth of pregnant females, neonatal death and neonatal sudden death, and those as described in the part of “Saccharide related diseases” of the application. With the similar MOA as described in the preceding embodiments, the same compositions of the present disclosure can be effective in treating one or more symptoms of the adverse reactions of other vaccines (e.g. influenza vaccines).

### Infection-Relating Autoimmune Diseases and Inflammation

As described herein, the methods of the present disclosure are effective against a wide range of infection-relating autoimmune diseases, particularly those caused by pathogenic antibodies in an individual. For example, a composition consisted of N-acetylneuraminic acid methyl ester and N-acetylneuraminic acid had demonstrated efficacy in a collagen-induced arthritis model (FIG. 10), which is known in the art as a commonly studied autoimmune model of rheumatoid arthritis (Brand, D. D. et al. Nat. Protoc. 2:1269-1275; 2007).

With the similar MOA as described in the preceding embodiments, the same compositions of the present disclosure can be effective in treating one or more symptoms of the other infection-relating autoimmune diseases preferably caused by pathogenic antibodies as describe in the part of “Saccharide related diseases” of the application. In some embodiments, the individual has GBS. In some embodiments, the individual has Bell's palsy. In some embodiments, the individual has a systematic inflammation or injury of kidney, cardiovascular, neural system, liver or/and digestive system. In some embodiments, the individual has COVID-19 long haulers. In some embodiments, the individual has abortion, or postpartum labor, or still birth of a pregnant female, or neonatal death and neonatal sudden death, related to infections or vaccinations.

### Cytokine Storm or CRS

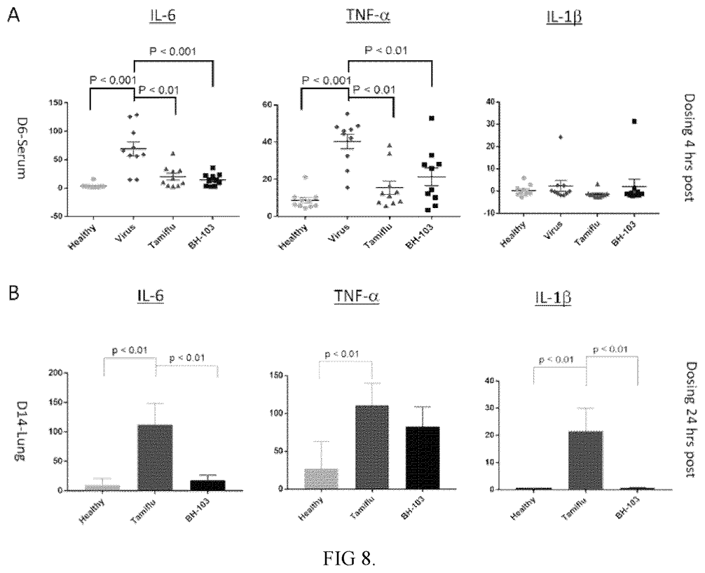

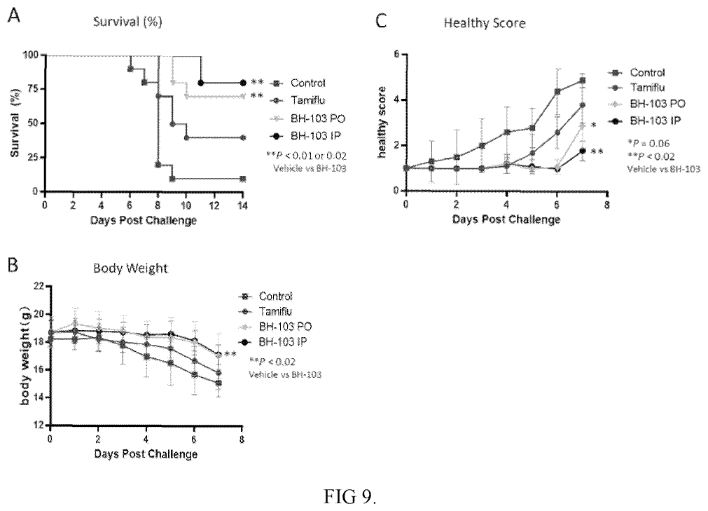

As described herein, the methods of the present disclosure are effective against a wide range of cytokine storm or CRS, particularly those caused by pathogenic antibodies in an individual. For example, the compositions consisted of N-acetylneuraminic acid methyl ester and N-acetylneuraminic acid are effect on decreasing inflammatory cytokine levels in mouse models as described in the Exemplification and shown in FIG. 8 and FIG. 14.

With the similar MOA as described in the preceding embodiments, the same compositions of the present disclosure can be effective in treating one or more symptoms of cytokine storm or CRS particularly those caused by pathogenic antibodies or therapeutic antibodies as describe in the part of “Saccharide related diseases” of the application. In some embodiments that may be combined with any of the preceding embodiments, the individual has cytokine storm or CRS during an infection. In some embodiments, the individual has cytokine storm or CRS during an adoptive T-cell therapy (e.g. CAR-T therapy). In some embodiments, the individual has cytokine storm or CRS during a treatment of an antibody medication.

### Gastrointestinal Diseases

As described herein, the methods of the present disclosure are effective against a wide range of gastrointestinal diseases in an individual. As is known in the art, many gastrointestinal diseases may present symptoms in tissues including without limitation the small and large intestines, mouth, stomach, esophagus, and anus. In some embodiments, the individual has a gastrointestinal disease caused by a viral infection. Viruses known to cause gastrointestinal disease may include without limitation rotaviruses, noroviruses, adenoviruses, and astroviruses. In some embodiments, the viral infection is a rotaviral infection. In some embodiments, the individual has acute infectious gastroenteritis.

In some embodiments, the individual has inflammatory bowel disease. An inflammatory bowel disease of the present disclosure may be chronic or acute. In some embodiments, the individual has Crohn's disease. In some embodiments, the individual has ulcerative colitis. In some embodiments, an inflammatory bowel disease may include colitis (such as diversion, lymphocytic, collagenous, or indeterminate colitis) or Behcet's disease.

### Dehydration

In another embodiment the present invention discloses the methods of using ORS compositions or ORS products for preventing and treating dehydration due to diarrhea or fever or rotavirus infection of humans and animals as mentioned in the part of “Saccharide related diseases” of the application.

One aspect of the methods is consisted of preparing the oral rehydration solution and orally administrating the rehydration solution to a human or an animal individual at risk of suffering or developing dehydration or rotavirus infection, or to the pregnant or feeding females with their fetus or sucking babies at risk of suffering or developing dehydration or rotavirus infection. Another aspect of the methods is consisted of intravenously administering a sterilized rehydration solution to a human or an animal individual at risk of suffering or developing dehydration or rotavirus infection, or to the pregnant or feeding females with their fetus or sucking babies at risk of suffering or developing dehydration or rotavirus infection.

In some embodiments, the ORS compositions comprise the combination of suitable amounts of the pharmaceutically acceptable salts and at least one of an analog of the sialic acid (e.g. N-acetylneuraminic acid methyl ester), a sialic acid (e.g. N-acetylneuraminic acid), another saccharide (e.g. galactose or lactose), a saccharide modifying molecule (e.g. methionine), and other optional components of the present disclosure if necessary.

In some embodiments, an ORS product is in a form of a powder mixture or a solution, wherein the pH of the ORS products when dissolved is between 3.0-6.8, preferably 3.5-6.0, preferably 4.0-5.5, most preferably 4.5-5.0, some embodiments, the effective dosages of the ORS compositions or ORS products for the uses as mentioned above are from about 0.01 mg/kg to 200 mg/kg of an analog of a sialic acid (e.g. N-acetylneuraminic acid methyl ester), or a sialic acid (e.g. N-acetylneuraminic acid), or another glycan (e.g. galactose or N-acetylglucosamine), or a saccharide modifying molecule (e.g. methionine), about 0.005 mg/kg to 100 mg/kg of a pharmaceutically acceptable salts (e.g. sodium citrate, or sodium acetate). In some embodiments, the effective dosages of an ORS solution for treating dehydration or rotavirus infection are from about 1 ml/kg to 100 ml/kg.

In some embodiments, the administrating routes of an ORS product include but not limited to oral or intravenous, or other using forms well known to those of ordinary skill in the pharmaceutical arts.

In some embodiments, a patient or an individual include but not limited to a human or an animal. In some embodiments, the humans include but not limited to males and females, newborns, 1-12 months old infants, 1-18 years old children, adults, old people, pregnant and feeding females, or the pregnant or feeding females with their fetus or suckling babies at risk of developing saccharide related diseases. In some embodiments, the animals include but not limited to livestock animals including but not limited to cows, pigs, horses, sheep or goats, llamas, cattle, donkeys; poultry including but not limited to chickens, ducks, gooses, turkeys and pigeons; companion animals including but not limited to dogs, cats, rodent pets and avian pets. More specifically, the livestock animals include but not limited to males and females, adult animals, newborn animals, infant animals, and other young age animals, pregnant and feeding female animals.

Combination Uses with Existing Therapeutics

In another embodiment the present invention discloses the methods of the combination use of an existing (e.g. Tamiflu or an antibiotic) or new therapeutic (known or unknown) with the compositions or the products disclosed in the present application as described in any of the preceding embodiments, for the prevention and treatment of saccharide related diseases of any of the preceding embodiments. In certain embodiments, the combination uses increase the efficacy or reduce the toxicity or side effects of a therapeutic (e.g. an antibiotic or a monoclonal antibody).

In some embodiments that may be combined with any of the preceding embodiments, the compositions or the products of the present disclosure are used for the prevention and treatment of saccharide related diseases of any of the preceding embodiments as: 1) therapeutic products; 2) food or feed additives or products comprising the additives; 3) dietary supplement products to help supporting or enhancing the protective structure or function of cells or tissues in particular respiratory or/and gastrointestinal tracts of humans and animals; and 4) oral rehydration salt or oral rehydration solution (ORS) for the prevention and treatment of dehydration due to diarrhea or fever of humans or animals.

One aspect of the methods is consisted of administrating a composition or a therapeutic product of any of the preceding embodiments and another therapeutic product (known or unknown) simultaneously to a human or an animal individual at risk of suffering or developing saccharide related diseases of any of the preceding embodiments; or to the pregnant or feeding females with their fetus or sucking babies at risk of suffering or developing saccharide related diseases of any of the preceding embodiments.

In some embodiments that may be combined with any of the preceding embodiments, the pH of the compositions or the products when dissolved is between 3.0-6.8, preferably 3.5-6.0, preferably 4.0-5.5, most preferably 4.5-5.0.

In some embodiments that may be combined with any of the preceding embodiments, the effective dosages of the compositions or the products of the present disclosure are from about 0.01 mg/kg to 200 mg/kg of an analog of a sialic acid (e.g. N-acetylneuraminic acid methyl ester), or a sialic acid (e.g. N-acetylneuraminic acid), or another glycan (e.g. galactose or N-acetylglucosamine), or a saccharide modifying molecule (e.g. methionine), about 0.005 mg/kg to 100 mg/kg of a pharmaceutically acceptable salts (e.g. sodium citrate, or sodium acetate).

In some embodiments, the effective dosages of the other therapeutics can be increased, equal or reduced (e.g. reducing from 10% to 90% of the amount of an antibiotic) when it is used in combination with a composition or a therapeutic product of the present disclosure.

In some embodiments, existing or new therapeutics (known or unknown) include but not limited to products consisted of chemicals (e.g. antibiotics), biologicals (e.g. antibodies, proteins and blood products), and plants or herbs, etc. without limitation. Examples of existing or new therapeutics include but not limited to Tamiflu, antibiotics or other anti-infective (e.g. antibodies), anti-virus, anti-inflammation, anti-allergy, anti-autoimmune diseases, anti-oncological diseases, anti-gastrointestinal diseases, anti-respiratory diseases, anti-cardiovascular diseases, anti-neurological diseases, anti-urological diseases, anti-reproductive diseases, anti-endocrine diseases, and any other known or unknown therapeutics without limitation.

In some embodiments, the two therapeutic products can be provided to an individual including the pregnant or feeding females by a variety of routes such as subcutaneous, topical with or without occlusion, oral, intramuscular, intravenous (both bolus and infusion), intraperitoneal, intracavity, or transdermal, inhalant, or other using forms well known to those of ordinary skill in the pharmaceutical arts.

In some embodiments that may be combined with any of the preceding embodiments, the saccharide related diseases particularly those caused by pathogenic antibodies, include but not limited to at least one of infectious diseases, infection-relating diseases, complications and sequela of infections, COVID-19 long haulers, cytokine storm and cytokine release syndrome (CRS), adverse reactions of vaccines or therapeutic antibodies, inflammation, inflammatory respiratory diseases, inflammatory gastrointestinal diseases, autoimmune diseases, allergy and cancers; preferably a saccharide related disease caused by a pathogenic pathogen or vaccines relating to the pathogen. The saccharide related diseases further include but not limited to abortion, postpartum labor, still birth of pregnant females, neonatal death and neonatal sudden death, caused by an infection or by a vaccine.

In some embodiments that may be combined with any of the preceding embodiments, the individual has serious infectious diseases particular respiratory viral infections (e.g. COVID-19 infection or avian influenza infection). In some embodiments that may be combined with any of the preceding embodiments, the individual has serious complications and sequela of infections including COVID-19 long haulers particularly those caused by respiratory viral infections (e.g. COVID-19 infection or avian influenza infection). In some embodiments that may be combined with any of the preceding embodiments, the individual has ARDS or ARD, cytokine storm or CRS, or/and systematic inflammation or injury of lung, kidney, liver, cardiovascular system, neural system, and digestive system during an infection. In some embodiments, the individual has cytokine storm or CRS during an adoptive T-cell therapy (e.g. CAR-T therapy). In some embodiments that may be combined with any of the preceding embodiments, the individual has cytokine storm or CRS during a treatment of an antibody medication.

In some embodiments that may be combined with any of the preceding embodiments, the individual has serious adverse reactions of vaccines, particularly those caused by COVID-19 vaccines or influenza vaccines. In some embodiments that may be combined with any of the preceding embodiments, the individual has abortion, or postpartum labor, or still birth of pregnant females, or neonatal death and neonatal sudden death, caused by an infection or by a vaccine.

### VII. Methods of Manufacture

In another embodiment the present invention discloses methods of making the compositions or products of any of the preceding embodiments. In some embodiments, the compositions or products are made by comprising an analog or a derivative of a sialic acid alone or the analog of a sialic acid in conjunction with at least one of the other major components of the present disclosure. The major components of the present disclosure include but not limited to: 1) a derivatives or an analogs of a sialic acid (e.g. N-Acetylneuraminic acid methyl ester) 2) a sialic acids including but not limited to N-acetylneuraminic acid, 2-Keto-3-deoxynononic acid, N-Acetylglucosamine, N-Acetylgalactosamine, N-Acetylmannosamine, and N-Glycolylneur-aminic acid; 3) another saccharides including but not limited to fructose, glucose, mannose, fucose, xylose, galactose, lactose; 4) a saccharide modifying molecules including but not limited to sulfur-containing amino acids (e.g. methionine and methionine-zinc complex); and 5) nutritional or pharmaceutically acceptable salts include but not limited to sodium chloride, potassium chloride, sodium citrate, sodium acetate, or the oral rehydration salts recommended by WHO. In some embodiments that may be combined with any one of the preceding embodiments, the pH of the compositions or the products or the therapeutic medicines when dissolved is between 3.0-6.8. In further aspects, the pH of the compositions or the products or the therapeutic medicines when dissolved is between 3.5-6.0, preferably 4.0-5.5, most preferably 4.5-5.0.

One aspect of the methods is consisted of manufacture of a product or a composition by combining suitable amounts of an analog of sialic acid (e.g. N-acetylneuraminic acid methyl ester) alone, or the analog of sialic acid plus at least one of the other major components (e.g. a sialic acid or/and methionine) of the present disclosure, and other optional components or materials known in the art if necessary, to form a tablet, a capsule, a pill, a powder mixture, a granule, an elixir, a tincture, a solution, a suspension, a syrup or a emulsion, a nasal drop or spray, an injectable, an infusion, or a form conjugated to a nano-particle, or other using forms well known to those of ordinary skill in the relevant arts; wherein the pH of the compositions or the products or the therapeutic medicines when dissolved is between 3.0-6.8. In further aspects, the pH of the compositions or the products or the therapeutic medicines when dissolved is between 3.5-6.0, preferably 4.0-5.5, most preferably 4.5-5.0.

In some embodiments that may be combined with any one of the preceding embodiments, the composition or the product comprises about 0.01 mg/ml to about 20 mg/ml, or about 0.01 mg/g to about 900 mg/g of a derivative or an analog of a sialic acid (e.g. a N-acetylneuraminic acid methyl ester), or a a sialic acid (e.g. N-acetylneuraminic acid), or another saccharide (e.g. a galactose or N-acetylglucosamine), or a saccharide modifying molecules (e.g. a methionine), about 0.005 mg/ml to about 10 mg/ml or about 0.005 mg/g to 500 mg/g of a citrate (e.g. a sodium citrate) or an acetate (e.g. a sodium acetate).

In another embodiment that may be combined with any one of the preceding embodiments, the present invention discloses a method of making an oral rehydration salt or a rehydration solution (ORS). In some embodiments that may be combined with any one of the preceding embodiments, the method comprises the combination of 3.5 grams of sodium chloride, 1.5 grams of potassium chloride, 2.9 grams of sodium citrate, 16-20 grams of glucose, and suitable amounts of an analog of sialic acid alone, or an analog of sialic acid (e.g.

N-acetylneuraminic acid methyl ester) plus at least one of the other major components (e.g. a sialic acid) of the present disclosure, to form a powder formula or a mixture of an oral rehydration salts (ORS) for one liter of water. In some embodiments, the amount of glucose of the ORS mixture can be adjusted (e.g. reduced). In some embodiments, the N-acetylneuraminic acid is used as an acidic reagent to achieve a desired pH value of an ORS solution.

In some embodiments that may be combined with any one of the preceding embodiments, the method comprises the combination of the ORS mixture and suitable amounts N-acetylneuraminic acid methyl ester. In some embodiments, the method comprises the combination of the ORS mixture and suitable amounts of N-acetylneuraminic acid methyl ester plus N-acetylneuraminic acid. In some embodiments, the method comprises the combination of the ORS mixture and suitable amounts of N-acetylneuraminic acid methyl ester, N-acetylneuraminic acid, and methionine. In some embodiments, the method comprises the combination of the ORS mixture and suitable amounts of N-acetylneuraminic acid and methionine. In some embodiments, N-acetylneuraminic acid can be used as an acidic reagent to achieve a desired pH value of an ORS. In some embodiments, the ORS powder mixture is dissolved in one liter of sterilized water, and the pH of the ORS solution is between 3.0-6.8, preferably between 3.5-6.0, preferably 4.0-5.5, most preferably 4.5-5.5.

In further embodiments, the ratios of N-acetylneuraminic acid methyl ester to methionine of the ORS mixture are 0.2-1:1, preferably 0.5-1:1. In some embodiments, the ratios of N-acetylneuraminic acid to methionine of the ORS mixture are 0.2-1:1, preferably 0.5-1:1. In certain embodiments, the ratios of N-acetylneuraminic acid methyl ester to N-acetylneuraminic acid to methionine of the ORS mixture are 0.2-1:0.2-1:1-2 (N-acetyl-neuraminic acid methyl ester:N-acetylneuraminic acid:methionine), preferably 0.5-1:1:2.

Another aspect of the method comprises dissolving the ORS powder mixture of any of the preceding embodiments, in suitable amount of sterilized water to form a sterilized oral rehydration solution, or a sterilized rehydration solution capable of being used by intravenously administration. In some embodiments, the pH of the rehydration solution is between 3.0-6.8, preferably between 3.5-6.0, preferably 4.0-5.5, most preferably 4.5-5.0.

In some embodiments, an ORS solution comprises about 0.001 mg/ml to about 1 mg/ml of an analog of a sialic acid (e.g. N-acetylneuraminic acid methyl ester), or a sialic acid (e.g. N-acetylneuraminic acid), or another saccharide (e.g. galactose or N-acetylglucosamine), or a saccharide modifying molecules (e.g. methionine).

In some embodiments that may be combined with any one of the preceding embodiments, an ORS powder mixture in a total amount of 28-30 grams for making 1000 ml of ORS solution, comprise about 0.01 gram to about 1.0 grams of an analog of the sialic acid (e.g. N-acetylneuraminic acid methyl ester), about 0.005 gram to 1.0 grams of N-acetylneuraminic acid, or a saccharide modifying molecule (e.g. methionine). In some embodiments, the pH of the solution of the ORS mixture is between 3.0-6.8, preferably between 3.5-6.0, preferably 4.0-5.5, most preferably 4.5-5.0.

### VIII. Kits

Certain aspects of the present disclosure relate to kits containing a pharmaceutical composition of any of the preceding embodiments. In some embodiments that may be combined with any one of the preceding embodiments, the kits contain an analog of sialic acid (e.g. N-acetylneuraminic acid methyl ester) alone, or the analog of a sialic acid in conjunction with at least one of the other major components of the present disclosure (e.g. N-acetylneuraminic acid). In some embodiments, the at least one of other major component is N-acetylneuraminic acid or methionine. In some embodiments, the kits may further comprise a citrate (e.g. sodium citrate) or an acetate (e.g. sodium acetate). In some embodiments, the kits may further include instructions for administering an effective amount of the pharmaceutical composition to an individual for preventing infectious diseases, infection-relating diseases, adverse reactions of vaccines or pathogenic antibodies. These instructions may refer to instructions customarily included in commercial packages of medicaments that contain information about the indications, usage, dosage, administration, contraindications, other medicaments to be combined with the packaged product, and/or warnings concerning the use of such medicaments, etc.

Suitable packages or containers for a kit of the present disclosure include, for example, packing bags, bottles, vials (e.g., dual chamber vials), syringes (such as single or dual chamber syringes) and test tubes. The article of manufacture may further comprise a label or a package insert, which is on or associated with the container, may indicate directions for reconstitution and/or use of the formulation. The label or package insert may further indicate that the formulation is useful or intended for injection or other modes of administration for preventing infectious diseases in an individual. The article of manufacture may further include other materials desirable from a commercial, therapeutic, and user standpoint, including other buffers, diluents, filters, needles, syringes, and package inserts with instructions for use.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXEMPLIFICATION

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: The Stability of N-Acetylneuraminic Acid or N-Acetylneuraminic Acid Methyl Ester at Different pH Conditions

Using simulated gastric fluid (SGF, pH=1.0), simulated intestinal fluid (SIF, pH=7.3) and phosphate buffer (PBS, pH=4.5) as diluents, the solution of N-acetylneuraminic acid (NANA) or N-acetylneuraminic acid methyl ester (NANA-Me) at the concentration of 5 mg/ml was prepared. The stabilities of N-acetylneuraminic acid, N-acetylneuraminic acid methyl ester and a combination of NANA plus NANA-Me (ratio=1:1) at 37° C. and different pH conditions were measured at various time points of 0.5, 1, 2, 4, and 6 hours. In addition, the optimal ratio of NANA to NANA-Me that maximizes the stability of NANA-Me at different pH conditions was tested at various time points of 0.5, 1, 2, 4, and 6 hours.

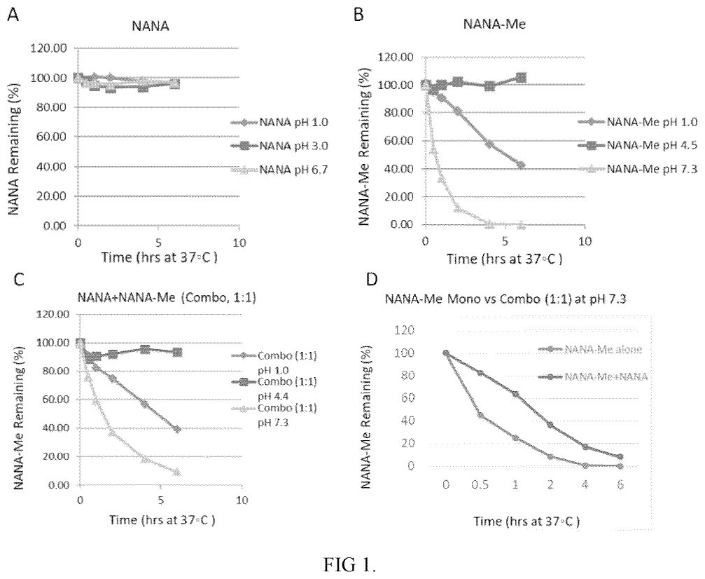

The results indicated that N-acetylneuraminic acid (NANA) was stable at all the pH conditions (FIG. 1A). N-acetylneuraminic acid methyl ester (NANA-ME) was most stable at about pH 4.5 and least stable at about pH 7.3 (FIG. 1B).

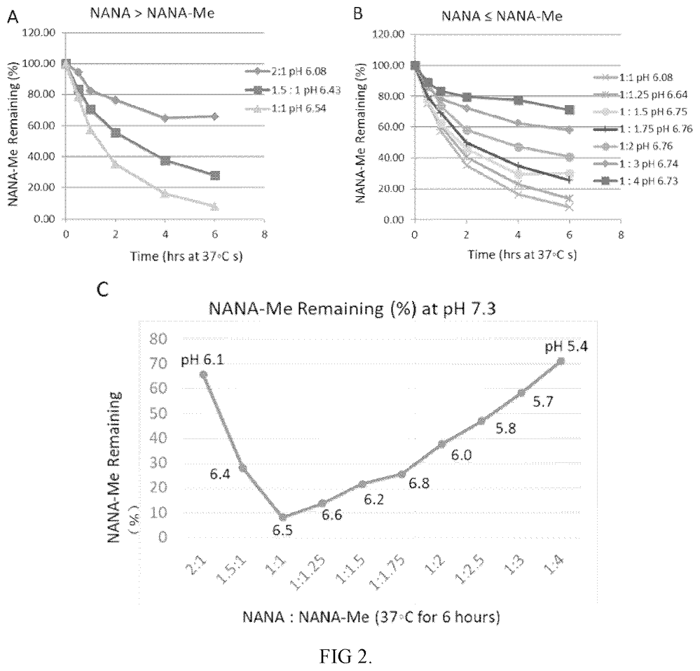

The stability of N-acetylneuraminic acid methyl ester (NANA-ME) was improved when it existed in a composition consisted of N-acetylneuraminic acid (NANA) and NANA-Me at the ratio of NANA-Me:NANA=1:1 (FIGS. 1C and 1D). When NANA≥NANA-Me, increase of NANA stabilized NANA-Me with the NANA-Me stability of 2:1≥1.5:1≥1:1 (NANA:NANA-Me) (FIG. 2A). When NANA<NANA-Me, increase of NANA did not stabilize NANA-Me with the NANA-Me stability of 1:2≥1:1.75≥1:1.5≥1:1.25≥1:1 (NANA:NANA-Me) (FIG. 2B). The optimal ratio of NANA:NANA-Me that maximizes the stability of NANA-Me at pH 7.4 is shown in FIG. 2C. The results indicated that the NANA-Me was most stable at NANA:NANA-Me of 1:4 and least stable at NANA:NANA-Me of 1:1 (FIG. 2C).

### Example 2: Repair of the Sialic Acid on Cell Surface by NANA-Me

Lung epithelium cell line A549 was cultured at 37° C. with freshly made solutions of NANA-Me or NANA with various concentrations for over night, and the sialic acid levels on the cells were determined with fluorescent labeled-wheat germ agglutinin (WGA) which specifically binds to sialic acid, and flow cytometry next day. The sialic acid levels of the A549 cells treated with NANA-Me increased in a way of dose dependent between the range of 1 μg/ml-50 μg/ml, while the sialic acid levels of the A549 cells treated with NANA were not changed significantly (FIGS. 3A and 3B). The data suggests that N-acetylneuraminic acid methyl ester (NANA-ME) is helpful with the synthesis and expression of the sialic acid on lung epithelium cells.

In another test, the A549 cells were cultured at 37° C. with freshly made solutions (pH=4.5) of NANA-Me and NANA with various ratios at 50 μg/ml of NANA-Me for over night, and the sialic acid levels on the cells were determined next day with the same method as described above. The results showed that the sialic acid levels of the A549 cells treated with the compositions comprising NANA and NANA-Me at the ratios of 1:1.25 or 1:2 (NANA:NANA-Me) were higher compared with the buffer (vehicle) treated control cells (FIG. 3C).

Thus a formulation consisted of NANA-Me and NANA at a ratio of 2:1 and pH 4.79 was prepared and named as BH-103 (or BH-103.3). In order to induce damaged cells, the A549 cells were treated with neuraminidase or sialidase (Roche, Shanghai) according to manufacturer's instruction. The sialidase treated A549 cells were cultured without or with 50 μg/ml of BH-103.3 at 37° C. for overnight, and the sialic acid level on A549 cells were determined next day. As shown in FIG. 3D, the sialic acid levels of the sialidase treated A549 cells was lower than that of untreated cells indicating loss of sialic acid on A549 cells after being digested with sialidase. The sialic acid levels of the A549 cells treated with sialidase and BH-103.3 was higher than that of untreated control cells.

Taken together, the data of the in vitro analysis indicated that N-acetylneuraminic acid methyl ester has the potential to enhance the expression of sialic acid and to repair the missed sialic acid on the A549 cell surface. The best enhancing or repairing effect of N-acetylneuraminic acid methyl ester could be achieved at pH 4.5 or in combination with N-acetylneuraminic acid at the ratios of NANA:NANA-Me being 1:1.25 or 1:2. The repairment of sialic acid on cell surface is helpful for recovery of damaged (e.g. infected or inflammatory) cells, blocking the self-attacking of immune system and reducing the severity of diseases including deaths. This is one of the mechanism of action (MOA-1) of N-acetylneuraminic acid methyl ester, or the compositions or the products containing N-acetylneuraminic acid methyl ester, for the treatment and prevention of the saccharide related diseases preferably caused by missed sialic acid on a cell surface. The saccharide related diseases include but not limited to infectious diseases, infection-relating diseases, complications and sequela of infections, COVID-19 long haulers, cytokine storm and cytokine release syndrome (CRS), adverse reactions of vaccines or therapeutic antibodies, inflammation, inflammatory respiratory diseases, inflammatory gastrointestinal diseases, infection-relating autoimmune diseases, allergy and cancers; preferably a saccharide related disease caused by a highly pathogenic virus or vaccines relating to the virus, as described in any of the preceding embodiments. The saccharide related diseases further include abortion, postpartum labor, still birth of pregnant females, and neonatal death and neonatal sudden death, caused by an infection or by a vaccine.

### Example 3: Blocking the Entry of COVID-19 Virus

The angiotensin converting enzyme 2 (ACE2) is an entry receptor for SARS-CoV-2, the virus responsible for the coronavirus disease 19 (COVID-19). The NB4 cell line, derived from a human acute promyelocytic leukemia, expressing ACE2 was used in an in vitro assay for the viral entry study of the SARS-CoV-2 (oCOVID-19) virus. Sialic acid as a component of ACE is responsible for the attachment of a coronavirus to ACE2 (X. Huang, et al. 2015. J Virology). The receptor binding domain (RBD) of the spike protein (S-RBD) of the SARS-CoV-2 virus is responsible for the entry of the COVID-19 virus into the host cells. The recombinant protein of S-RBD of the COVID-19 virus (with human Fc as a tag for detection) was purchased from Sino Biological (Beijing).

In one test, the NB4 cells were cultured at 37° C. with the formula of BH-103.3 (pH 4.79) as described above, at 50 μg/ml of NANA-Me for overnight, and the sialic acid levels on the cells were determined next day with the same method as described in Example 2. The results showed that the sialic acid levels on the NB4 cells treated with BH-103.3 were higher compared with untreated control cells (FIG. 4A), indicating the enhancing effect of BH-103.3 on the sialic acid expression of the NB4 cells.

In a parallel test the NB4 cells with or without treatment of BH-103.3 were incubated with the recombinant S-RBD of the COVID-19 virus in ice for one hour and then with a fluorescent (PE) labeled anti-human Fc antibody, followed by a flow cytometry analysis. The results showed that the S-RBD level on the NB4 cells treated with BH-103.3 was lower compared with the control cells without BH-103.3 treatment (FIG. 4B).

In another test, NB4 cells were treated with neuraminidase (sialidase) (Roche, Shanghai) according to manufacturer's instruction. The sialidase treated NB4 cells were cultured without or with 50 μg/ml of BH-103.3 at 37° C. for overnight, and the sialic acid level on the NB4 cells were determined next day. In a parallel test the binding of the recombinant S-RBD of the COVID-19 virus to the NB4 cells with or without treatment of BH-103.3 were tested with the same analysis as described above. As shown in FIG. 4C, the sialic acid levels of the sialidase treated NB4 cells was lower than that of untreated cells indicating loss of sialic acid on A549 cells after being digested with sialidase. The sialic acid levels of the NB4 cells treated with sialidase and BH-103.3 was higher than that of control cells treated with sialidase alone. Nevertheless, 89% of the binding of the COVID-19 S-RBD to the NB4 cells treated with sialidase and BH-103.3 was reduced compared to the control cells treated with sialidase alone (FIG. 4D). This result indicated that replacement of N-acetylneuraminic acid by N-acetylneuraminic acid methyl ester induced a structural or chemical modification of the viral receptor that significantly decreased the binding affinity of the COVID-19 S-RBD.

The effect of the entry blocking of COVID-19 S-RBD by BH-103.3 was also tested with HEK-293 cells, a cell line derived from human embryonic kidney cells, and the similar results are shown in FIGS. 4E and 4F.

Taken together, the data shown above indicated that N-acetylneuraminic acid methyl ester can repair the sialic acid of the ACE2 receptor on the NB4 cell (MOA-1). On the other hand, the replacement of N-acetylneuraminic acid methyl ester induces a structural or chemical modification of the viral receptor that significantly decrease the binding affinity of the COVID-19 S-RBD. Therefore, although the sialic acid expression on the BH-103.3 treated NB4 cells was higher the S-RBD binding was significantly lower especially with the damaged cells with missed sialic acid, since there was more replacement of N-acetylneuraminic acid methyl ester on the damaged cells. The results indict that N-acetylneuraminic acid methyl ester (or BH-103) can chemically modify the sialic acid of the SARS-CoV-2 viral receptor and blocking viral entry into host cells (MOA-2). Based on the MOA-2, N-acetylneuraminic acid methyl ester can prevent the COVID-19 infection by blocking viral entry into host cells, and treat the infection by blocking viral spread into new cells.

Because sialic acid is a receptor component for not only coronavirus but also other viruses (e.g. influenza viruses or rotavirus) the receptor modification and blocking entry by N-acetylneuraminic acid methyl ester should be widely effective for the prevention and treatment of other infections caused by other viruses using sialic acids as receptors (e.g. influenza viruses or rotavirus). This is another MOA (MOA-2) of N-acetylneuraminic acid methyl ester, or the compositions or the products containing N-acetylneuraminic acid methyl ester such as the BH-103.3 for the treatment and prevention of viral infections, particular the highly pathogenic viral infections such as COVID-19 or an avian influenza infection.

### Example 4. Compositions for the Treatment of Influenza Infection

Mouse models of C57BL/6J aged 6-8 weeks with highly pathogenic influenza viral infection are used to validate therapeutic efficacy of the compositions comprising N-acetylneuraminic acid methyl ester. The mice were inoculated via oral and nasal administration with the influenza virus of A/PR/8/34(H1N1) strain, or the A/H3N2/Hong Kong/1/68 strain at the concentration capable of inducing 90% of death. The course of the mouse model was 2 weeks. Within week one post infection, the mice looked sick, did not eat well and failed to gain weight as quickly as healthy mice. Some mice had ARDS symptoms such as severe shortness of breath or labored and unusually rapid breathing. By the second week, about 80-90% of mice with serious illness died. Animal body weight and clinical signs were observed and record every day. The clinic symptoms were scored in some test. An effective result of a treatment was judged by reduced severity of clinic symptoms and deaths, stable body weight or/and better healthy scores (the lower the better).

In one experiment, C57BL/6J mice were randomly divided into five groups, infected with the A/PR/8/34(H1N1) influenza virus and treated orally at 4 hours post infection with compositions consisted of: 1) saline (model control); 2) NAAN-Me (pH 1.5), 30 mg/kg; 3) NAAN-Me (pH 7.0), 30 mg/kg; 4) NANA-Me+NANA (1:1, pH 5.0), 30 mg/kg; and 5) NANA-Me+NANA (1:1, pH 6.0), 15 mg/kg. Each group was dosed once per day for 10 days. Animal body weight and clinical signs were recorded and observed every day up to day 14. The survival rates of each group are shown in FIG. 5A. The results showed that the treatment of the compositions comprising NANA-Me and NANA at either 30 mg/kg and pH 5.0 or 15 mg/kg and pH 6.0, significantly reduced deaths (P<0.05). However, NANA-Me was ineffective at 30 mg/kg with neither pH 1.5 nor pH 7.0 for the treatment of the influenza infection.

In another experiment, C57BL/6J mice were randomly divided into four groups, infected with the A/PR/8/34 (H1N1) influenza virus and treated orally at 24 hours post infection with: 1) phosphate buffer (PBS, pH 4.5); 2) NANA-Me in PBS (pH 3.5), 15 mg/kg; 3) NANA-Me in PBS (pH 4.5), 15 mg/kg; and 4) NANA-Me+NANA in pH 4.5 PBS (1:1, pH 3.6), 15 mg/kg. Each group was dosed once per day for 10 days. Animal body weight and clinical signs were recorded and observed every day up to day 14. The survival rates of each group are shown in FIG. 5B.

The results showed that the treatment of NANA-Me at 15 mg/kg and pH 4.5 significantly reduced deaths (P<0.05), while the treatment of NANA-Me at pH 3.5 was ineffective although with the same dosage (15 mg/kg). In addition, the compositions comprising NANA-Me and NANA was effective at 15 mg/kg and pH 3.6 (P<0.05).

Taken together, the data of this in vivo study showed that N-acetylneuraminic acid methyl ester was effective at pH 4.5-6.0 and ineffective at pH 7.0 or pH <3.5. However N-acetylneuraminic acid methyl ester was effective at pH 3.6 when it co-existed with N-acetylneuraminic acid together, indicating that N-acetylneuraminic acid could improve the stability and efficacy of N-acetylneuraminic acid methyl ester at lower pH condition.

### Example 5. A Formulation for H1N1 Influenza Infection

A formulation consisted of NANA-Me plus NANA at the ratio of 1:1 and pH 4.5 was prepared and named as BH-103.1.

C57BL/6J mice were randomly divided into three groups, infected with the A/PR/8/34(H1N1) influenza virus and treated orally at 4 hours post infection with: 1) phosphate buffer (PBS, pH 4.5), vehicle control; 2) Tamiflu, 30 mg/kg, drug control; 3) BH-103.1 (pH 4.5), 30 mg/kg. Each group was dosed once per day for 10 days. Animal body weight and clinical signs were recorded and observed every day up to day 14. At day 6 post infection some mice had symptoms of ARDS and lost body weight more than 25%. The mice were judged death according to the procedure and were sacrificed. The tissues of lung, heart, brain, kidney, liver, and intestines of those mice were collected for histological evaluation, bloods were collected and sera were isolated for cytokine detection. The results of survival rates and body weight are shown in FIG. 6A. When the mice were treated at 4 hours post infection, the effect of BH-103.1 treatment was equivalent to that of Tamilu treatment Both of BH-103.1 and Tamiflu significantly reduced the deaths (P<0.05) and maintained better body weight compared to control mice (FIG. 6A).

In another experiment, three random groups of the A/PR/8/34(H1N1) infected mice were treated orally at 24 hours post infection with: 1) phosphate buffer (PBS, pH 4.5), vehicle control; 2) Tamiflu, 15 mg/kg, drug control; 3) BH-103.1 (pH 4.5), 15 mg/kg. Each group was dosed once per day for 10 days. Animal body weight and clinical signs were recorded and observed every day up to day 14. At the end of the course tissues of lung, heart, brain, kidney, liver, and intestines were collected from at least 3 mice for histological evaluation. The tissue lysates of mouse lungs were prepared from lung samples collected by snap frozen in liquid nitrogen.

The survival rates and body weight of each group are shown in FIG. 6B. When the mice were treated at 24 hours post infection the treatment of the BH-103.1 at 15 mg/kg reduced deaths (P<0.05) and maintained better body weight (FIG. 6B) compared to vehicle controls. However, the treatment of Tamilu at 24 hours post infection did not show significant effect in this animal model.

### BH-103.1 Significantly Reduced the Inflammation of Lung and Other Organs

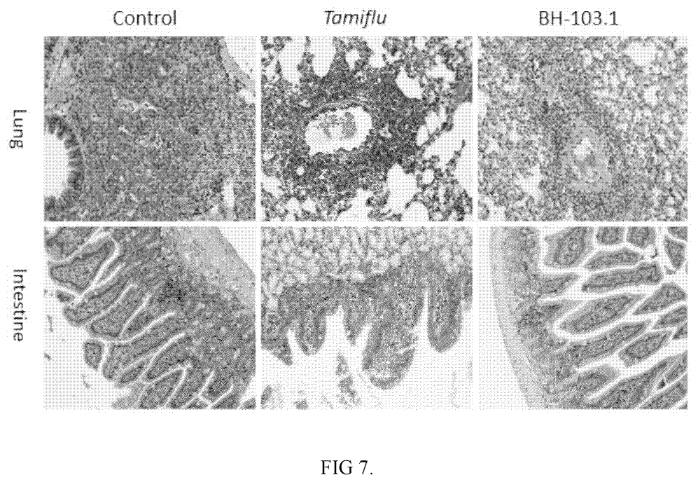

Severe inflammation of lung and intestine at day 6 post infection was observed with the hematoxylin-cosin (HE) stained tissue sections from the mice of vehicle control (FIG. 7A) and Tami/lu group (FIG. 7B). The inflammation of those organs from the mice of BH-103.1 treatment was lighter (FIG. 7C). The lung lesion included pulmonary congestion, alveolar epithelial hyperplasia and thickening, alveolar atresia, alveolar dilatation and alveolar fusion, and infiltration of inflammatory cells.

### BH-103.1 Significantly Reduced Cytokine Secretion

Mouse cytokine levels of IL-1β, TNF-α and IL-6 of the mouse sera collected at day 6 post infection, and the tissue lysates of mouse lungs collected at day 14 as described above, were determined using a CBA kit (BD Biosciences) according to manufacturer's instruction. The results are summarized in FIG. 8. When the mice were treated 4 hours post infection, the treatment of either BH-103.1 or Tamiflu at 30 mg/kg significantly reduced the cytokine level of IL-6 (P<0.001) and TNF-α (P<0.01) at day six post infection compared to those of the viral control group (FIG. 8A).

When the mice were treated at 24 hours post infection with the BH-103.1 at 15 mg/kg, the lung cytokine level of IL-6 (P<0.01) and TNF-α (P<0.01) and IL-1β (P<0.01) were recovered to the levels of healthy mice at day 14 post infection. However, the three cytokine levels of the mice treated with Tamilu at 24 hours post infection remained abnormally higher compared to those of the healthy mice (P<0.01) (FIG. 8B). The results indicated that BH-103.1 could reduce the cytokine levels better than Tami/lu after 4 hours of an influenza infection. This result indicated that BH-103.1 or the compositions or products comprising N-acetylneuraminic acid methyl ester and N-acetylneuraminic acid are effective for preventing or treating cytokine storm or CRS by inhibition of cytokine production.

### Example 6. A Formulation for H3N2 Influenza Infection

A formulation consisted of NANA-Me and NANA at 1.25:1 (pH 4.5) was prepared and named as BH-103.2.

C57BL/6J mice were randomly divided into four groups, infected with the A/H3N2/Hong Kong/1/68 influenza virus and treated at 8 hours post infection with: 1) phosphate buffer (PBS, pH 4.5), orally, vehicle control; 2) Tamilu, 30 mg/kg, orally, drug control; 3) BH-103.2 (pH 4.5), 30 mg/kg, orally (PO); and 4) BH-103.2 (pH 4.5), 30 mg/kg, intraperitoneal injection (IP). Each group except Tamiflu group was dosed once per day for 10 days. Tamilu group was dosed once per day for 5 days. Animal body weight and clinical signs were recorded and observed every day up to day 14.

The survival rates, healthy scores and body weight of each group are shown in FIG. 9. The results showed that the treatment of the BH-103.2 at 30 mg/kg via either oral or intraperitoneal injection, significantly reduced deaths (P<0.02 or P<0.01) (FIG. 9A), maintained better healthy scores (P<0.02 or P=0.06) (FIG. 9B) and body weight (P<0.02) (FIG. 9C) compared to vehicle controls. In addition, the therapeutic efficacy of BH-103.2 for the treatment of the influenza infection was better than that of Tamiflu since Tamiflu did not show significant efficacy compared to vehicle controls in this animal model.

In summary, the data of the in vivo studies further support the MOAs of N-acetylneuraminic acid methyl ester for the treatment and prevention of saccharide related diseases as described above in Example 2-3. The in vivo results further indicated that the compositions or products comprising N-acetylneuraminic acid methyl ester or N-acetylneuraminic acid methyl ester and N-acetylneuraminic acid at the pH of 3.5-6.0, preferably 4.0-5.5, mostly preferably 4.5-5.0 when dissolved, are effective for the treatment of highly pathogenic influenza infections. The compositions or the products comprising N-acetylneuraminic acid methyl ester can be also effective for other viral infections including COVID-19 infection, based on their MOAs. Further, the compositions or the products comprising N-acetylneuraminic acid methyl ester or N-acetylneuraminic acid methyl ester and N-acetylneuraminic acid, can be effective for other saccharide related diseases of any of the preceding embodiments, include but not limited to infectious diseases, infection-relating diseases, complications and sequela of infections, COVID-19 long haulers, cytokine storm and cytokine release syndrome (CRS), adverse reactions of vaccines or therapeutic antibodies, inflammation, inflammatory respiratory diseases, inflammatory gastrointestinal diseases, infection-relating autoimmune diseases, allergy and cancers; preferably a saccharide related disease caused by a highly pathogenic virus or vaccines relating to the virus. The saccharide related diseases further include abortion, postpartum labor, still birth of pregnant females, and neonatal death and neonatal sudden death, caused by an infection or by a vaccine.

### Example 7. A Formulation for Collagen-Induced Arthritis (CIA)

A rat model of collagen-induced arthritis (CIA) which is known in the art as a commonly studied autoimmune model of rheumatoid arthritis (see, e.g., Brand, D. D. et al. (2007) Nat. Potoc. 2:1269-1275), was used to validate therapeutic efficacy of BH-103.1 as described above. Lewis rats at about eight weeks old were immunized once per week for three weeks using bovine type II collagen as an immunogen. The paw volume and body weight were measured twice a week; and representative images were taken once per week. Disease activity is determined by measuring inflammation swelling in the affected joints (paw volume or thickness) over time.

In one experiment, the rats with significant inflammatory swelling joints were randomly divided into two groups: 1) control, not treated, n=5; and 2) treated with BH-103.1 (NANA:NANA-Me=1:1, pH 4.5) orally at 1.5 mg/kg, once every other day for 15 days, n=5. The paw volume and body weight were measured twice a week; and representative images were taken once per week. FIG. 10A shows representative gross images taken at day 5 (after 2 dosing). All control rat ankles and knees of control rats were swelling, red and hot. All control rats walked with dragged legs. As shown in FIG. 10B, treatment with BH-103.1 significantly reduced the inflammatory severity of rat ankles and knees compared to those of controls. In addition, treatment with BH-103.1 maintained better body weight compared to those of controls (FIG. 10C).

The data indicated that the compositions or the products comprising N-acetylneuraminic acid methyl ester are effective for the prevention and treatment of rheumatoid arthritis, and can be widely effective for other autoimmune diseases preferably caused by pathogenic antibodies and/or missing sialic acid as described in the current disclosure.

### Example 8. A Formulation for the Treatment of Serious Adverse Reactions Caused by Anti-Coronavirus Antibodies

In PCT/US2014/25918 (Biological therapeutics for infectious or inflammatory diseases or conditions), the pathogenic role of anti-influenza sera was disclosed using a timed-pregnant mouse model. The similar mouse model was used in the current application to evaluate the pathogenic role of anti-coronavirus antibodies and the therapeutic effect of the formulation of BH-103 for the treatment of the disorders caused by the pathogenic anti-coronavirus antibodies.

### Antibodies Against the Spike Protein of Coronavirus Caused Serious Adverse Reactions

The anti-coronavirus antibodies of rabbit polyclonal antibodies specific to the recombinant spike one (S1) or nucleocapsid (N) proteins of SARS-CoV-2 virus, recombinant spike glycoprotein of SARS-CoV virus and mouse monoclonal antibody specific to the recombinant nucleocapsid (N) protein of SARS-CoV virus were purchased (Bioss Antibodies, Beijing). Naturally occurred human monoclonal antibodies specific to the receptor binding domain (RBD) of the spike protein one (S1) of the SARS-CoV-2 virus, isolated from patients with COVID-19 infection were provided by HuaAn McAb Biotech (Hangzhou) for research use only. The naturally occurred human monoclonal antibodies specific to the COVID-19 (SARS-CoV-2) S1 protein included antibodies of B38 (Wu et al., Science 368, 1274-1278; 2020), Regn10987 (Hansen et al., Science 369, 1010-1014; 2020), CC12.3 (Yuan et al., Science 369, 1119-1123; 2020), and Cr3022-b6 (bioRxiv preprint doi: https://doi.org/10.1101/2020.12.14.422791).

SPF-grade C57BL/6J pregnant mice at pregnancy (embryonic) day E13-E14 were purchased from Shanghai SLAC Laboratory Animal Co., Ltd. The animals were randomly divided into groups as needed, two pregnant mice for each group at every experiments. The purified IgGs of the rabbit anti-COVID-19 (SARS-CoV-2) S1, anti-COVID-19 N, anti-SARS S, anti-SARS N, human monoclonal anti-COVID-19 S1 antibodies of B38 and Regn10987 as described above were used in the virus-free pregnant mouse model. The purified IgG of sera from healthy rabbit, mouse and human as well as the anti-COVID-19 S1 monoclonal antibody of Cr3022-b6, were used as controls. Two dosages of each antibody IgG were injected intraperitoneally (IP) into timed-pregnant mice twice every three days at pregnancy (embryonic) day of E15 (about 26-28 g) and E18 (about 30-32 g) respectively (FIG. 11A). For each polyclonal antibody, 50 μg (microgram) for the first dose (about 2.0 mg/kg) and 60 μg for the second dose (about 2.0 mg/kg) were administrated. For each monoclonal antibody, 40 μg for the first dose (about 1.5 mg/kg) and 50 μg for the second dose (about 1.5 mg/kg) were administrated. The body weight of the pregnant mice was measured every day before and after the antibody injection. The mouse pups were born at about E20-E21 and the healthy status including clinical signs of the newborn mouse pups were observed and recorded. The course was ended at day 1 or 2 post birth. At the end day, the blood samples were collected from newborn mouse pups, and the sera were isolated and stored at −80° C. for cytokine detection. The tissue samples of lungs, hearts, brains, kidneys, livers, and intestines were collected from at least 3 mouse pups, fixed in formalin for 48-72 hours, went through gradient alcohol dehydration and embedded in paraffin, and tissue sections were processed for histological evaluation and immunofluorescent staining.

Injection of the Regn10987 antibody into pregnant mice induced significant fetal death and neonatal death of the mouse pups delivered to the dames (p value: 0.02) (Table 1). The fetal death was confirmed by autopsy (FIG. 11B). The frequencies of the sick and death of the fetus and newborn mouse pups are summarized in FIG. 11C and Table 1. The results with this virus-free animal model indicated that the monoclonal antibody of Regn10987 is at the highest risk for inducing sick and death (61.9%), followed by the monoclonal antibody of B38 (45.8%) and the polyclonal anti-COVID-19 S1 (45.5%). The polyclonal anti-SARS S also caused significant sick and death of the fetus and newborn mouse pups (37.6%). In addition, hyperemia at the end of left up and down limbs and a small hemangioma at the side of left eye of one pup was observed. The pup was delivered to a deme injected with the polyclonal anti-COVID-19 S1 antibody. Neither the control antibodies nor the anti-COVID-19 N nor the anti-SARS N antibodies caused significant sick and death of the newborn mouse pups (Table 1).

### Systemic Inflammation of Multiple Organs

The tissue sections of lung, brain, heart, kidney, intestine and liver of the newborn mouse pups were stained with hematoxylin-cosin (HE) for histology evaluation. The human IgG or rabbit IgG bund on the tissues in vim was detected by an immunofluorescent staining with fluorescent labeled anti-human IgG or anti-rabbit IgG antibodies.

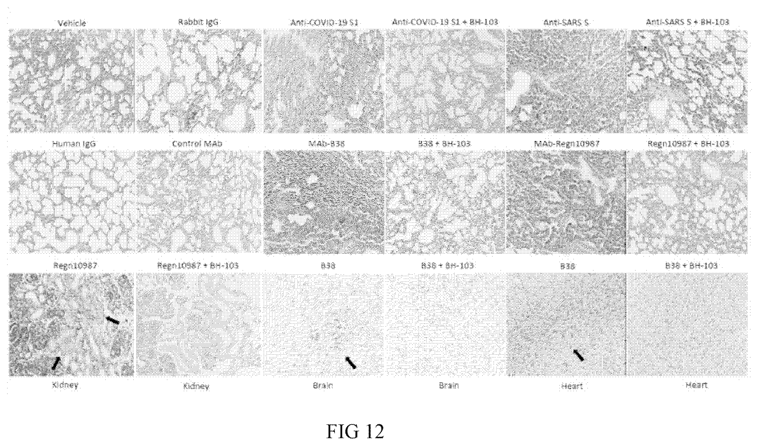

Lung inflammation and injury Acute lung inflammation and injury were observed with the HE stained tissue sections from the mouse pups delivered to the dames injected with the anti-COVID-19 S1, anti-SARS S, and the antibodies of Regn10987 and B38 (FIG. 12). The lung lesion included pulmonary congestion, alveolar epithelial hyperplasia and thickening, hemorrhage, alveolar atresia, alveolar dilatation and alveolar fusion. Infiltration of inflammatory cells and hemorrhage at the local lesion areas were observed. There were not significant or minor histological changes with the lungs from the pups delivered to the dames injected with the antibodies of anti-COVID-19 N, anti-SARS N, CR3022-b6, and the control IgGs of human, rabbit and mouse.

Other organ inflammation Inflammatory reactions and hemorrhage were also observed with the tissues of kidney, brain and heart from the mouse pups as mentioned above.

The histology of the kidneys from the mouse pups delivered to the dames with the injection of anti-COVID-19 S1, anti-SARS S, B38 and Regn10987 showed acute tubular injury. Renal tubular epithelial cells showed granular or vacuolar degeneration, dilated or obstructed lumen, and some of the epithelial cells fell off, renal interstitial edema with a small amount of inflammatory cells infiltration (FIG. 12). The kidney injury caused by antibody Regn10987 was the most significant (FIG. 12). Small amount of cerebral hemorrhage or inflammatory cells infiltration was observed with the brain from a mouse pup delivered to a dame with injection of antibody B38 (FIG. 12C). Large amount of inflammatory cells infiltration was observed with the brains from mouse pups delivered to the dames with injection of antibody SARS S and Regn10987. Further, myocardial hemorrhage was observed with the hearts form the mouse pups delivered to the dames with injection of antibodies of anti-SARS S and B38. Myocardial swelling and inflammatory cells infiltration were observed with a mouse pup delivered to the dame with injection of antibodies of B38 (FIG. 12).

### Injected Antibodies Detected at Diseased Tissues

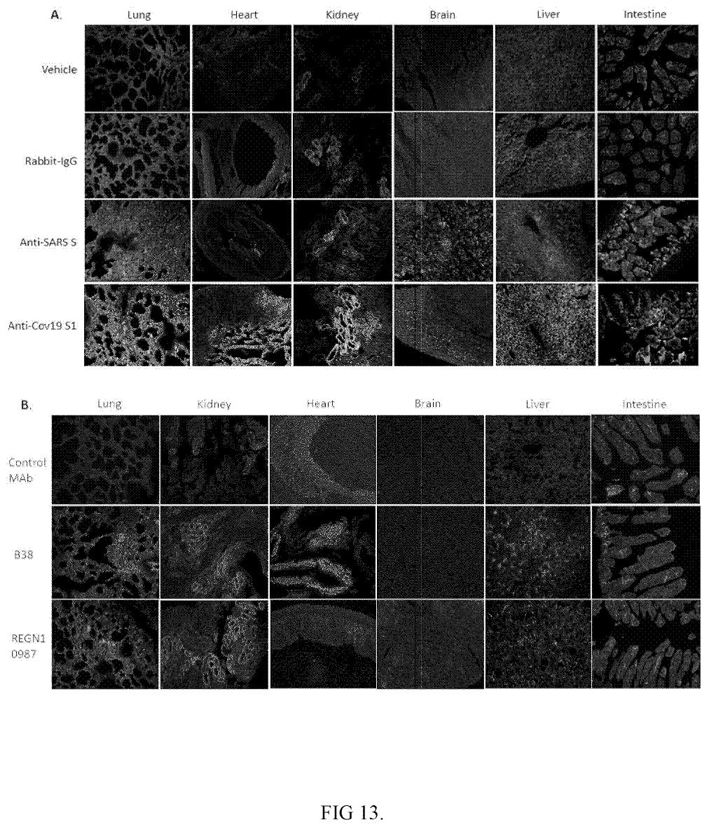

As an evidence of pathogenic antibodies, the human IgG or rabbit IgG was detectable at the inflammatory areas of lungs, kidneys, hearts, brains, livers and intestines of mouse newborns (FIG. 13) by an immunofluorescent staining as described above. The results provided evidence for the in viv binding of the pathogenic antibodies such as anti-SARS-CoV-2 spike antibodies to fetal tissues, activating self-attack immune responses and inducing systematic inflammation and damages of multiple organs including lung, kidney, hart brain, liver and intestine.

In summary, certain antibodies against the spike protein of the SARS-CoV-2 virus are pathogenic and induce serious conditions during the COVID-19 infection. The pathogenic antibodies can be induced during an infection (e.g. the COVID-19 or an influenza infection) or a vaccination (e.g. the COVID-19 or an influenza vaccination), or passively introduced (e.g. a therapeutic antibody). The diseases or conditions caused by pathogenic antibodies include infectious diseases, infection-relating diseases, complications and sequela of infections, COVID-19 long haulers, cytokine storm and cytokine release syndrome (CRS), adverse reactions of vaccines or therapeutic antibodies, inflammation, inflammatory respiratory diseases, inflammatory gastrointestinal diseases, infection-relating autoimmune diseases, allergy and infection-relating cancers, and any other disorders (known or unknown) inducible by pathogenic antibodies. The diseases or conditions caused by pathogenic antibodies further include abortion, postpartum labor, still birth of pregnant females, and neonatal death and neonatal sudden death, caused by an infection or by a vaccine.

### The Prevention and Treatment of the Adverse Reactions of Pathogenic Antibodies

As shown in FIG. 12, the treatment of BH-103.3 (pH 4.5, 15 mg/kg) at the same time with the antibody injection of anti-COVID-19 S1 or anti-SARS S, significantly decreased the severity of lung inflammation compared to the mouse pups delivered to the dames injected with either of the antibody alone (FIG. 12A). Similar therapeutic effect of BH-103.3 (pH 4.5, 15 mg/kg) administrated at the day before the antibody injection was also observed. Similar therapeutic effect of BH-103.3 (pH 4.5, 15 mg/kg) at either day before or the same time with the antibody injection of the antibody of Regn10987 or B38 was also observed (FIG. 12) (Table 1). The treatment of BH-103.3 (pH 4.5, 15 mg/kg) at either day before or the same time with the antibody injection also significantly decreased the severity of the inflammation of other organs beside lung compared to the mouse pups delivered to the dames injected with either of the antibody alone (FIG. 12).

Taken together, the in vivo data indicated that the compositions or products comprising N-acetylneuraminic acid methyl ester or N-acetylneuraminic acid methyl ester and N-acetylneuraminic acid (e.g. BH-103.3) are effective for the prevention and treatment of the inflammation of multiple organs caused by pathogenic antibodies, and can be widely effective for other inflammatory diseases preferably caused by pathogenic antibodies and/or missed sialic acid as described in the current disclosure.

TABLE 1
The sick and death rates of mouse pups delivered to the dames
with the injection of anti-coronavirus antibodies

| | | | | Sick + | | | |
|---|---|---|---|---|---|---|---|
| | | Sick | Death | Death | Odds | 95% | P |
| Injected IgG of | N= | (%) | (%) | (%) | Ratio | CI | value |
| Saline | 17 | 0 | 5.88 | 5.88 | NA | NA | NA |
| Healthy rabbit | 6 | 0 | 16.7 | 16.7 | 3.20 | 0.17-61 | 1.00 |
| serum | | | | | | | |
| Healthy mouse | 7 | 0 | 11.1 | 11.1 | 2.67 | 0.14-50 | 1.00 |
| serum | | | | | | | |
| Healthy human | 14 | 0 | 7.14 | 7.14 | 1.23 | 0.07-22 | 1.00 |
| serum pool* | | | | | | | |
| Anti-COVID-19 N | 13 | 7.69 | 0 | 7.69 | 1.33 | 0.08-24 | 1.00 |
| Anti-COVID-19 S1 | 22 | 27.3 | 18.2 | 45.5 | 13.3 | 1.5-119 | 0.01 |
| Anti-COVID-19 | 11 | 9.09 | 0 | 9.09 | 1.55 | 0.09-27 | 1.00 |
| S1 + BH-103 | | | | | | | |
| Anti-SARS N | 15 | 6.67 | 13.3 | 20.0 | 4.00 | 0.37-43 | 0.30 |
| Anti-SARS S | 14 | 31.3 | 6.25 | 37.6 | 12.0 | 1.2-117 | 0.03 |
| Anti-SARS | 16 | 6.25 | 6.25 | 12.5 | 2.29 | 0.19-28 | 0.60 |
| S + BH-103 | | | | | | | |
| MAb-Cr3022-b6 | 9 | 0 | 11.1 | 11.1 | 2.00 | 0.11-36 | 1.00 |
| MAb-B38 | 24 | 33.3 | 12.5 | 45.8 | 13.5 | 1.5-119 | 0.01 |
| B38 + BH-103 | 13 | 7.69 | 0 | 7.69 | 1.33 | 0.08-24 | 1.00 |
| MAb-Regn10987 | 35 | 17.1 | 44.8 | 61.9 | 21.3 | 2.5-179 | 0.006 |
| Regn10987 + | 14 | 7.14 | 0 | 7.14 | 1.23 | 0.07-22 | 1.00 |
| BH-103 | | | | | | | |

*Normal IgG pool of 4 healthy individuals without infection or vaccination of coronavirus

### BH-103.3 Significantly Reduced Cytokine Production

The sera from newborn pups as mentioned above were tested for inflammatory cytokines of MCP-1, TNF-α, IL-4, IL-6 and IL-10 using a 5-plex multiplex Luminex assay kit (Millipore) according to manufacturer's instruction. The results are summarized in FIG. 14, the treatment of BH-103.3 significantly reduced the cytokine level of MCP-1 (P<0.001) and IL-4, compared to the mouse pups delivered to the dames injected with the antibody alone. The data further support that the compositions or products comprising N-acetylneuraminic acid methyl ester and N-acetylneuraminic acid (e.g. BH-103.1 or BH-103.3) are effective for preventing or treating cytokine storm or CRS by inhibition of cytokine production.

### Example 9. Pathogenic Antibodies Bind to Damaged Cells

Binding of anti-coronavirus and anti-influenza antibodies to healthy (intact) or damaged lung epithelium cells was tested with the human lung epithelium cell line A549 and the in vitro assay as described in Example 2. The damaged A549 cells with missed sialic acid on the cell surface were used to imitate the in vivo conditions of infected lung epithelium cells (sick cells).

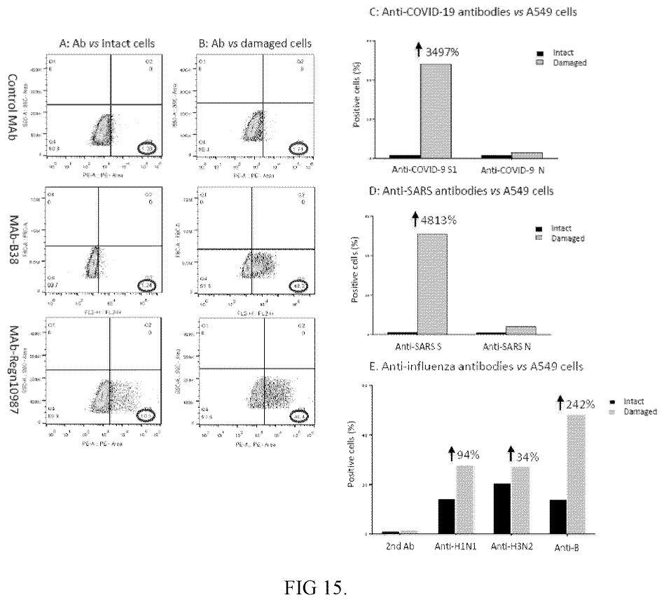

The two human monoclonal antibodies against the COVID-19 S-RBD protein, Regn10987 and B38, strongly bound to the damaged A549 cells with missed sialic acid on the cell surface. The Regn10987 also weakly bound to healthy A549 cells while the B38 did not bind to the healthy A549 cells. The control antibody of Cr3022-b6 did not bind to the healthy A549 cells nor the damaged cells (FIGS. 15A, 15B and 15C).

In addition, the antibodies against the spike glycoprotein of SARS-CoV virus (anti-SARS S) strongly bound to the damaged A549 cells with missed sialic acid (FIG. 15D) while neither of the antibodies bound to the healthy A549 cells with sialic acid. In addition, the polyclonal antibody against SARS-CoV-2 nucleocapsid protein (anti-COVID-19 N) and the antibody against SARS-CoV nucleocapsid protein (anti-SARS N) did not significantly bind to healthy nor damaged A549 cells (FIG. 15D).

Further, the antibodies of B38 and anti-SARS S strongly bound to the damaged human embryonic kidney HEK-293 cells with missed sialic acid. The two antibodies did not bind to the healthy HEK-293 cells. The antibodies of anti-COVID-19 N and the anti-SARS N did not bind to the healthy nor the damaged HEK-293 cells (data not shown).

Furthermore, the anti-influenza viral antibodies of anti-H1N1 (California/09), anti-H3N2 and anti-B virus also significantly bound to the damaged A549 cells with missed sialic acid, compared to the healthy A549 cells (FIG. 15E). The results are consistent to the in vivo observations of the pathogenic actions of anti-influenza sera in a timed-pregnant mouse model, published in PCT/US2014/25918 (Biological therapeutics for infectious or inflammatory diseases or conditions).

Taken together, the results of the in vitro analysis provide a possible mechanism of action of the pathogenic antibodies. The in vitro data indicated that certain antibodies against spike protein of SARS-CoV-2 virus and SARS-CoV virus have the potential to mislead the immune response to attack self by binding to the sick cells such as human lung epithelium cells, or human embryonic kidney cells with damaged suger chain on the cellular surface. This is consistant to the in vivo results as described in Example 8. The Regn10987 antibody may have higher risk potential to activate immune responses since the antibody bind to not only sick cells but also heathy cells despite at low rate. Similar pathogenic action was observed with the anti-influenza viral antibodies as well (probably related to the anti-HA antibodies), which is consistant to the in vivo observations published in PCT/US2014/25918 (Biological therapeutics for infectious or inflammatory diseases or conditions), in which the pathogenic role of anti-influenza sera was disclosed using a timed-pregnant mouse model. The compositions or products comprising N-acetylneuraminic acid methyl ester, or N-acetylneuraminic acid methyl ester and N-acetylneuraminic acid (e.g. BH-103.1, BH-103.2 or BH-103.3) are effective for preventing or treating the diseases or conditions caused by the pathogenic antibodies induced by coronaviruses and influenza viruses, as mentioned above.

### Example 10. Binding of Pathogenic Antibodies to Human Fetal or Disease Tissues

As evidence of the pathogenicity of the pathogenic anti-COVID-19 S1 antibodies, in vivo antibody binding of the human and rabbit anti-COVID-19 S1 antibodies were significantly detectable at the inflammatory and lesion areas of the tissues of lungs, kidneys, brains, hearts, livers, and intestines from mouse pups with server sickness, using fluorescent labeled anti-human or anti-rabbit secondary antibody and an immunofluorescent staining (FIGS. 13A and 13B).

Further, the Regn10987 antibody from COVID-19 infected patient and fetal tissues or multiple diseased tissues of tissue array slides (purchased from US Biomax) were used to evaluate the pathogenicity of the antibody specific to the SARS-CoV-2 S1 protein. The Regn10987 antibody bound to the tested multiple human fetal tissues of lung, heart, kidney, brain, pancreas, liver, thymus and testicle (FIG. 16), indicating that the unmatured fetal tissues are vulnerable to the pathogenic antibody. In addition, the Regn10987 bound broadly to the human inflammatory tissues or cancer tissues of respiratory, cardiovesvular, urinary and digestive system (FIGS. 17A and 17B). The human inflammatory diseases tested include pneumonia, bronchitis, bronchiectasis, valvular disease, rheumatoid valvular disease, myocarditis, esophagitis, gastritis, collitis, appendicitis, pancreatitis, and hepatitis. The cancer tissues tested include small cell lung carcinoma, kidney clear cell carcinoma, and myxoma. The data indicate that the most of the inflammatory tissues or some of cancer tissues are vulnerable to the pathogenic antibody.

In summary, the pethogenic antibodies together with the damaged or inflammatory cells or tissues can be the cause of serious infections particularly highly pathogenic viral infections (e.g. COVID-19 infection), serious adverse reactions of vaccines (e.g. COVID-19 vaccines), serious complications of infections (e.g. ARDS), cytokine storm or CRS, infection-relating inflammation and autoimmune diseases, COVID-19 long haulers, and infection-relating cancers which can occur if an inflammatory cellular proliferation stimulated by a pathogenic antibody repeatedly persists for long time and loses control. Further, the pethogenic antibodies can bind to the unmatured fetal cells or tissues and cause abortions, postpartum labors, still births of pregnant females, and neonatal deaths and neonatal sudden deaths.

The compositions or products comprising N-acetylneuraminic acid methyl ester, or N-acetylneuraminic acid methyl ester and N-acetylneuraminic acid (e.g. BH-103.1, BH-103.2 or BH-103.3), are effective for preventing or treating the diseases or conditions caused by the pathogenic antibodies induced by coronaviruses and influenza viruses, as mentioned above.

### Example 11. Prevention and Treatment Respiratory Diseases with Formulations

### Preparation of Formulations

A powder mixture of a formula named as BH-103.4 was made by mixing 1.0 gram of N-acetylneuraminic acid methyl ester, 0.75 gram of N-acetylneuraminic acid, 0.75 gram of sodium citrate, and 2.5 grams of glucose. One pack of the BH-103.4 was dissolved in 100 ml of sterilized water (10 mg/ml of NANA-Me), and the pH was adjusted to 4.5.

Another formula package of powder mixture named as BH-103.5 was made by mixing 6.0 grams of N-acetylneuraminic acid methyl ester, 4.0 grams of N-acetylneuraminic acid, and other additives up to 30 grams.

One pack of BH-103.5 was mixed with 1000 kg of nursery feed.

Treatment of Chicken Influenza Infection with Formula BH-103.4

Chickens aged 28 days (about 0.67 kg) suffered from co-infections of avian influenza and mycoplasma from an avian influenza outbreak at a chicken farm. 60 of sick chickens were divided into three groups, and treated orally with 1) untreated (n=20); 2) 1.5 ml (microliter) of Tamilu (22 mg/kg) plus Amikacin (according to manufacturer's instruction) (n=20); 3) 1.5 ml of BH-103.4 (22 mg/kg) plus Amikacin (according to manufacturer's instruction) (n=20). The three groups of chickens were treated once per day for 7 days.

19/20 (95%) of the chickens without treatment were died; 16/20 (80%) of the chickens treated with Tamilu plus antibiotic survived; and 18/20 (90%) of the chickens treated with BH-103.4 survived. The results indicated that the chickens with BH-103.4 treatment showed the best effect with lowest death rate as shown in Table 2.

TABLE 2
The death rates of chickens with influenza
infection and BH-103 treatment

| | | | Death | Odds | | |
|---|---|---|---|---|---|---|
| Treatment with | n= | Death | Rate (%) | Ratio | 95% CI | P value |
| Untreated | 20 | 19 | 95 | 76 | 7.69-750 | <.0001 |
| Tamiflu + | 20 | 4 | 20 | 0.25 | 0.001-0.13 | <.0001 |
| antibiotic* | | | | | | |
| BH-103.4 + | 20 | 2 | 10 | 0.11 | 0.001-0.07 | <.0001 |
| antibiotic | | | | | | |

*Amikacin.
Fisher Exact Probability Test, two tailed

Treatment of Chicken Infection of Newcastle Disease Virus with Formula BH-103.4

Chickens aged 55 days (about 1.7 kg) suffered from co-infections of Newcastle disease virus and mycoplasma at a chicken farm and the death rate was 90-100%. 80 of sick chickens were divided into four groups, and treated orally or intramuscular with 1) untreated (n=20); 2) 3.5 ml of BH-103.4 (20 mg/kg), oral (n=15); 3); 2) 3.5 ml of BH-103.4 (20 mg/kg), intramuscular (n=15); 4) 2.5 ml of BH-103.4 (20 mg/kg) plus ciprofloxacin lactate (according to manufacturer's instruction), oral (n=15); 5) 2.5 ml of BH-103.4 (20 mg/kg) plus ciprofloxacin lactate, intramuscular (n=15). The sick chickens were treated once per day for 7 days.

All the chickens treated with BH-103.4 showed significant decreased death rates (Table 3). The treatment of BH-103.4 via intramuscular showed better effect than the oral treatment. The best efficacy was observed with the combination treatment of BH-103.4 and ciprofloxacin lactate via intramuscular. The results are shown in Table 3.

TABLE 3
The death rates of chickens infected with
Newcastle disease virus and BH-103 treatment

| | | | Death | Odds | | |
|---|---|---|---|---|---|---|
| Treatment with | n= | Death | Rate (%) | Ratio | 95% CI | P value |
| Untreated | 20 | 19 | 95 | 52.3 | 5.17-528 | <.0001 |
| BH-103.4, PO* | 15 | 4 | 26.7 | 0.19 | 0.002-0.19 | <.0001 |
| BH-103.4, IM* | 15 | 1 | 6.67 | 0.004 | 0.0002-0.07 | <.0001 |
| BH-103.4 + | 15 | 1 | 6.67 | 0.004 | 0.0002-0.07 | <.0001 |
| antibiotic**, PO | | | | | | |
| BH-103.4 + | 15 | 1 | 6.67 | 0.004 | 0.0002-0.07 | <.0001 |
| antibiotic, IM | | | | | | |

*PO: oral, IM: intramuscular.
**Ciprofloxacin lactate.
Fisher Exact Probability Test, two tailed

Treatment of Pneumonia of Pigs with Formula BH-103.4

During an influenza outbreak season, the pigs aged 120-135 days (about 5-60 kg) suffered from pneumonia at a farm. The symptoms of the disease included fever (40° C.), dark fur, hard breath (abdominal breathing) and the decreased food intake. The mortality rate was about 5-10%. The sick pigs were treated with either florfenicol or BH-103.4 via feed mixture consisted of either florfenicol or BH-103.4 for 5-7 days. At the fourth day of treatment, the pigs with BH-103.4 treatment showed improved symptoms of normal body temperature (about 37° C.), glossy fur, easy breath and the increased food intake (from 1.5 kg/day/each to 2.0 kg/day/each). The results showed that the treatment of BH-103.4 significantly improved the symptoms and reduced the death of the sick pigs compared to untreated or antibiotic-treated controls as shown in Table 4.

TABLE 4
The death rates of pigs with influenza infection and pneumonia

| | | | Death | Odds | | |
|---|---|---|---|---|---|---|
| Treatment with | n= | Death | Rate (%) | Ratio | 95% CI | P value |
| Vehicle | 100 | 8 | 8.00 | 1.74 | 0.55-5.51 | 0.40 |
| Antibiotic* | 105 | 5 | 4.76 | 0.58 | 0.18-1.82 | 0.40 |
| BH-103.4 | 141 | 1 | 0.71 | 0.01 | 0.01-0.67 | <0.05 |

*Florfenicol.
Fisher Exact Probability Test, two tailed

Prevention of Respiratory Infections of Weaning Piglets with BH-103.5

From about 2-3 weeks of weaning, some piglets showed the symptoms of ARDS with difficult breath caused by respiratory infection. The sick rate was about 10% and the death rate was about 5%. The feed comprising formula of BH-103.5 as made above were tested for the prevention of respiratory infection of weaning piglets at age of 6-8 weeks. Control feed comprised 500 grams of Tilmicosin per 1000 kg. The nursery feeds were given to 763 of weaning piglets and 750 of controls from day 15 of the weaning period. The total feeding days with the nursery feeds were 4 days.

The piglets feed with the feed comprising BH-103.5 had less respiratory infections and lower death rate compared to the piglets feed with the feed comprising Tilmicosin (Table 5).

TABLE 5
The death rates of weaning piglets with respiratory
infections and the treatment of BH-103.5

| | | | Death | Odds | | |
|---|---|---|---|---|---|---|
| Treatment of | n= | Death | Rate (%) | Ratio | 95% CI | P value |
| Tilmicosin | 750 | 38 | 5.10 | 5.76 | 2.56-13.0 | <.0001 |
| BH-103.5 in feed | 763 | 7 | 0.92 | 0.17 | 0.08-0.39 | <.0001 |

Chi-square Test

In addition, the sick piglets of control group were treated with antibiotic (Tilmicosin). Some of the piglets treated with antibiotic grew slowly, looks smaller and not healthy while the sick piglets feed with the formulas did not lose body weight significantly and looked in a good healthy status after recovered.

The data indicated that feeding weaning piglets with the BH-103 containing feeds significantly reduced the frequencies of sick and death of weaning infants suffer from respiratory infection. Thus the products comprising N-acetylneuraminic acid methyl ester and N-acetylneuraminic acid can be used for the prevention of respiratory infections. The other diseases of weaning infants include but not limited to respiratory infections, ARDS, ARD, asthma, the infection of foot and mouth disease virus (FMDV), the infection of porcine circovirus (PCV) and other disorders of weaning infants.

In summary, the in vivo results on the field further indicated that the compositions or products comprising N-acetylneuraminic acid methyl ester or N-acetylneuraminic acid methyl ester and N-acetylneuraminic acid at the pH of 4.5-5.0, are effective for the prevention and treatment of respiratory infections including avian and pig influenza infections and pneumonia, and Newcastle disease virus infection. The compositions or the products comprising N-acetylneuraminic acid methyl ester can be widely effective for other viral infections including COVID-19 infection, based on their MOAs. In addition, better effect can be achieved by the combination treatment of BH-103.4 and an antibiotic.

The effective dosages of the formulas for the treatment of respiratory infections, are from about 0.01 mg/kg to about 100 mg/kg of N-acetylneuraminic acid methyl ester, or N-acetylneuraminic acid. The dose amount of an antibiotic can be reduced to 10-90% of the dosages recommended by manufactures when it is used in combination with a formulation or a product comprising N-acetylneuraminic acid plus N-acetylneuraminic acid methyl ester or N-acetylneuraminic acid plus N-acetylneuraminic acid methyl ester and N-acetylneuraminic acid.

### Example 12. Prevention and Treatment Gastrointestinal Diseases with Formulations

### Preparation of Formulations

One package of a regular oral rehydration salt mixture consisted of 3.5 grams of sodium chloride, 1.5 grams of potassium citrate, 2.9 grams of sodium citrate, and 20 grams of glucose in a total amount of 27.9 grams was dissolved in 1000 ml of sterilized water (ORS alone).

One package of powder mixture consisted of 1.0 gram of N-acetylneuraminic acid, 1.0 gram of methionine and 27.9 grams of oral rehydration salt mixture (formula BH-104.1) was dissolved in 1000 ml of sterilized water, and the pH of the solution was about 6.0.

One package of powder mixture consisted of 1.0 gram of N-acetylneuraminic acid methyl ester, 0.5 gram of N-acetylneuraminic acid, 1.0 gram of methionine, and 27.9 grams of oral rehydration salt mixture (formula BH-104.2) was dissolved in 1000 ml of sterilized water, and the pH of the solution was about 5.5-6.0.

The formulas of BH-104.1 and BH-104.2 and BH-103.4 as prepared above were used for the prevention and treatment of viral diarrhea or infective gastroenteritis of pigs.

### Treatment of Piglet Diarrhea and Gastroenteritis Caused by Viral Infections

Diarrhea is a common disorder with sucking or weaning piglets. Viral or bacterial infections are common causes of piglet diarrhea. Piglets with viral diarrhea usually have yellow watery stools and piglets with bacterial diarrhea usually have gray stools. Sometimes, yellow diarrhea is accompanied with vomiting showing the characteristic of infective gastroenteritis. Often, piglet diarrhea or infective gastroenteritis is caused by co-infection of viruses (e.g. porcine rotavirus, PEV or TGEV) and bacteria. Piglets younger than one week could have a serious viral diarrhea or infective gastroenteritis with high death rates from about 70% to over 90%.

The formulations of BH-104.1, BH-104.2 and BH-103.4 (pH 5.5-6.0 when dissolved) as prepared above were used for the treatment of diarrhea piglets at ages 1-10 days.

Newborn piglets aged 1-3 days post birth suffered from diarrhea caused by porcine rotavirus (PoRV) infection at a pig farm. The mortality rate was about 90% and the efficacy of antibiotic (Enrofloxacin) treatment was less than 30%. 600 of sick piglets were treated orally with the BH-104.1 solution (pH 5.5-6.0) at 1.0 ml/kg (1.0 mg/ml), once per day for 2-3 days. About 85% of the piglets treated with BH-104.1 stopped diarrhea within 24 hours after 1-2 doses of treatment and recovered after 2-3 doses (Odd Ratio: 0.01, 95% Confidence Intervals: 0.01-0.02, P<0.0001).

At another pig farm, newborn piglets aged 2-10 days post birth suffered from diarrhea and gastroenteritis caused by PoRV infection. The mortality rate was about 85% and the efficacy of antibiotic treatment was less than 30%. 42 of the sick piglets were treated orally with 200 ml/each of ORS solution containing 3-4 ml (mg) of the BH-104.2 (pH about 6.0), 26 of the sick piglets were treated orally with 200 ml/each of ORS solution containing 4-5 ml (mg) of the BH-103.4 (pH about 6.0 when dissolved), once per day for 2-3 days. The results showed that BH-104.2-ORS or BH-103.4-ORS was significant effective for treatment of piglet diarrhea and gastroenteritis caused by PoRV infection, as shown in Table 6.

TABLE 6
The death rates of pigs with PORV infection and the
treatment of ORS containg formulas

| | | | Death | Odds | | |
|---|---|---|---|---|---|---|
| Treatment of | n= | Death | Rate (%) | Ratio | 95% CI | P value |
| Untreated | 50, | 43 | 86.0 | 123 | 24-627 | <.0001 |
| | D2-D10 | | | | | |
| BH-104.2 in | 42, | 40 | 4.76 | 0.01 | 0.002-0.04 | <.0001 |
| ORS | D2-D4 | | | | | |
| BH-103.4 in | 26, | 23 | 11.5 | 0.02 | 0.01-0.09 | <.0001 |
| ORS | D5-D10 | | | | | |

Fisher Exact Probability Test, two tailed

At another pig farm, newborn piglets aged 1-3 days post birth suffered from diarrhea and gastroenteritis caused by TGEV infection. The mortality rate was about 80% and the efficacy of antibiotic treatment was less than 30%. 122 piglets were treated with BH-103.4 at 1.5 mg/kg, once per day. About 85% of the piglets treated with BH-103.4 stopped diarrhea and recovered after 2-3 doses of treatment (Odd Ratio: 0.03, 95% Confidence Intervals: 0.01-0.06, P<0.0001).

At another pig farm, newborn piglets suffered from diarrhea and gastroenteritis caused by PEDV infection. The mortality rate was about 25-30%. 364 of piglets were treated with BH-104.2 (pH 5.5-6.0 when dissolved) at 1.5 mg/kg, once per day at 1-2 days post birth. Another 310 of piglets were delivered to the dames treated with BH-104.2 orally dosing 5 times through feed plus water at 100 mg/dame every other day started from 5 days before delivery. All the piglets were observed up to 15 days post birth.

The death rate of the piglets treated with BH-104.2 or the piglets with BH-104.2-treated mothers significantly decreased compared to those of untreated (Table 7). The results indicated that BH-104.2 effectively prevented newborn piglet diarrhea by either treating the piglets or their mothers.

TABLE 7
The death rates of pigs with PEDV infection
and the treatment of BH-104.2

| | | | Death | Odds | | |
|---|---|---|---|---|---|---|
| Treatment of | n= | Death | Rate (%) | Ratio | 95% CI | P value |
| Untreated | 366 | 92 | 25.1 | 40.4 | 12.7-128 | <.0001 |
| BH-104.2 to | 364 | 3 | 0.82 | 0.03 | 0.01-0.08 | <.0001 |
| piglets | | | | | | |
| BH-104.2 to | 310 | 5 | 1.61 | 0.05 | 0.02-0.12 | <.0001 |
| mothers | | | | | | |

Fisher Exact Probability Test, two tailed

At another pig farm, newborn piglets suffered from diarrhea and gastroenteritis caused by PEDV infection. The mortality rate was about 20%. 400 of piglets were delivered to the dames treated orally with BH-103.4 (pH about 6.0 when dissolved) in a mixture of feed plus water, dosing 5 times at 100 mg/dame every other day, started from day 5 before delivery. All the piglets were observed up to 15 days post birth. The death rate of the piglets delivered to BH-103.4 treated dames significantly decreased compared to those of untreated (Table 8). The results indicated that BH-103.4 effectively prevented newborn piglet diarrhea by treating their mothers.

TABLE 8
The death rates of pigs with PEDV
infection and the treatment of BH-103.4

| | | | Death | Odds | | |
|---|---|---|---|---|---|---|
| Treatment of | n= | Death | Rate (%) | Ratio | 95% CI | P value |
| Untreated | 368 | 72 | 19.6 | 5.84 | 3.33-10.3 | <.0001 |
| BH-103.4 to | 400 | 16 | 4.00 | 0.17 | 0.10-0.30 | <.0001 |
| mothers | | | | | | |

Fisher Exact Probability Test, two tailed

Prevention of Respiratory Infections of Weaning Piglets with BH-104

From the first week of weaning, many piglets have diarrhea. The feed comprising the formula mixture of BH-104.1 and BH-104.2 as made above were tested for the prevention of gastrointestinal diseases of weaning piglets at age of 4-5 weeks. Regular nursery feed was used as control. Weaning piglets were feed with 1) Regular nursery feed, n=200, 2) BH-104.1 containing feed, n=208, and 3) BH-104.2 containing feed, n=203. The nursery feeds were given from day 1 to day 5 of the weaning period. The total feeding days with the nursery feeds were 5-10 days.

The piglets treated with the feed comprising either BH-104.1 or BH-104.2 looked healthy with pink skin and shining fur compared to the pigs of the control group. The diarrhea rates and the death rates of the weaning piglets with the formula treatment were significantly lower than those of the control group (Table 9). In addition, the diarrhea of the two treated group piglets was lighter (not water-like diarrhea) compared to the diarrhea of the control group piglets (water-like diarrhea).

The diarrhea piglets of control group were treated with antibiotic (Ofloxacin). Some of the piglets treated with antibiotic grew slowly, looks smaller and not healthy while the diarrhea piglets with the formulas were not treated, did not lose body weight significantly and looked in a good healthy status after recovered.

TABLE 9
The frequencies of diarrhea and death of weaning piglets

| | | | | Death | | | |
|---|---|---|---|---|---|---|---|
| | | | Rate | Rate | Odds | 95% | |
| Treatment of | n= | Diarrhea | (%) | (%) | Ratio | CI | P value |
| Untreated | 200 | 103 | 51.5 | 15.5 (31) | 21.0 | 10.5- | <.0001 |
| | | | | | | 42.1 | |
| BH-104.1 | 208 | 10 | 4.81 | 0.48 (1) | 0.05 | 0.02- | <.0001 |
| in feed | | | | | | 0.10 | |
| BH-104.2 | 203 | 7 | 3.45 | 0.00 (0) | 0.03 | 0.02- | <.0001 |
| in feed | | | | | | 0.08 | |

Fisher Exact Probability Test, two tailed

The effective dosages of the formulas for the treatment of piglet diarrhea and gastroenteritis are from 0.01 mg/kg to 20 mg/kg of N-acetylneuraminic acid or methionine or N-acetylneuraminic acid methyl ester.

In summary, the in vivo results on the field indicated that the compositions or products comprising N-acetylneuraminic acid and methionine, or N-acetylneuraminic acid methyl ester and N-acetylneuraminic acid and methionine, or N-acetylneuraminic acid methyl ester and N-acetylneuraminic acid, at the pH of 5.5-6.0 when dissolved, are effective for the prevention and treatment of gastrointestinal diseases such as diarrheas and gastroenteritis, preferably caused by infections such as a viral infection.

Prevention and Treatment of IBD with Formulation

The inventor-self suffered from inflammatory bowel disease (IBD) for over five years. During the last reoccurrence, the patient had serious bleeding diarrhea, and abdominal pain for couple of months. Treatment with antibiotic for two weeks was ineffective. Oral treatment with the BH-103.4 (consisted of N-acetylneuraminic acid methyl ester, N-acetylneuraminic acid and sodium citrate, pH 4.5 when dissolved) at 2 mg/kg once per day was performed. The symptoms of the IBD were improved after one week of treatment and recovered two weeks of treatment. However, when the treatment was stopped the IBD relapsed thus the treatment was restarted. The repeated treatment was lasted for about 9 months until the IBD symptoms completely disappeared and was not relapsed. The patient has been fine without relapse for over 11 months since the last treatment.

The result suggested that the formula Bh-103.4 consisted of N-acetylneuraminic acid methyl ester, N-acetylneuraminic acid and sodium citrate is effective for a chronic inflammation such as IBD.

### Example 13. Prevention and Treatment H9N2 Infection with Formulation BH-103

### Preparation of Formulations

A powder mixture of a formula named as BH-103.6 was made by mixing 1.0 grams of N-acetylneuraminic acid methyl ester, 0.5 gram of N-acetylneuraminic acid, 0.4 gram of sodium citrate. One pack of the BH-103.6 was dissolved in 100 ml of sterilized water (10 mg/ml of NANA-Me), and the pH was about 4.5.

Treatment of H9N2 Influenza Infection with Formula BH-103.6

SPF chicken embryos at embryotic day 14 (E14), 15 and 16 were treated via allantoic injection with 1) saline (n=12); and 2) BH-103.6 (50 mg/kg) (n=14). At E16 and 8 hours after dosing the chicken embryos were inoculated with H9N2 avian influenza virus. The allantoic fluid were collected from each embryo at 24 and 48 hours post virus challenge, and the viral titers were determined by hemagglutination inhibition (HI) test. The viral titer higher than 16 was count as positive.

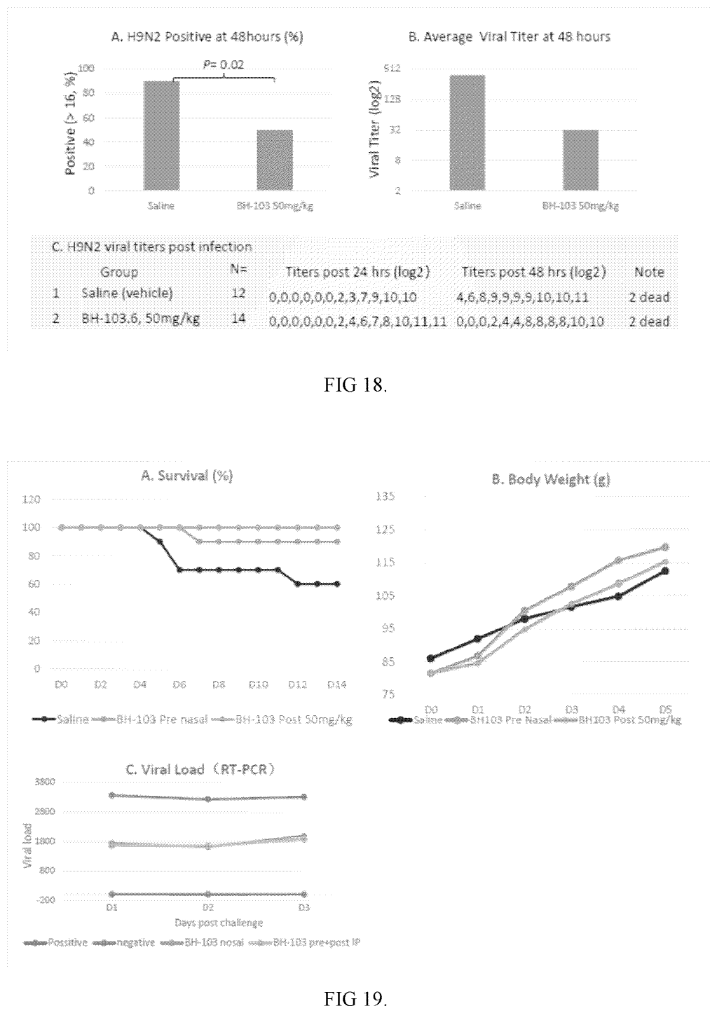

As shown in FIGS. 18A-18C, BH-103.6 pre-treatment decreased H9N2 viral infection of the chicken embryos from 90% to 50% (Fisher Exact Probability Test: p=0.02) (FIG. 18A) In addition, BH-103.6 pre-treatment decreased the viral load at about four log grades (FIG. 18B). The viral titer of each embryo at 24 and 48 hours are shown in FIG. 18C.

### Example 14. Prevention and Treatment of Avian Coronavirus Infection with Formulation BH-103.6

The formulation BH-103.6 as mentioned above (pH 4.5 when dissolved) was used to prevent and treat the infection of an avian coronavirus, avian infectious bronchitis virus (IBV) in a chicken model.

SPF chicks at the age of day 9 (D9) were randomly divided into three groups and treated once per day with: 1) saline alone (vehicle control), 2 days pre-infection (D-2) to DO, nasal and eye dropping, and D1-D7, D9, D11, D13, intraperitoneal injection (IP); 2) BH-103.6 (pH 4.5), D-2-D0 (viral challenge day), 0.5 mg, nasal and eye dropping; and 3) DO-D7, D9, D11, D13, 30 mg/kg, intraperitoneal injection (IP). At DO and 8 hours after dosing of groups 1-2, the chicks were inoculated with the IBV via nasal and eye dropping. Group 3 chicks were dosed 4 hours post viral challenge. Animal body weight and clinical signs were recorded and observed every day up to day 14.

The survival rates and body weight of each group are shown in FIGS. 19A-19C. The results showed that either pre-treatment of the BH-103.6 via nasal and eye dropping or intraperitoneal injection (IP), or treatment of the BH-103.6 post viral infection (p<0.5, Fisher Exact Probability Test) reduced deaths, loss of body weight and viral load compared to vehicle controls in this animal model. The data indicated that BH-103.6 can effectively prevent and treat a coronavirus infection such as IBV infection.

Other embodiments besides the above may be articulated as well. The terms and expressions therefore serve only to describe the disclosure by example only and not to limit the disclosure. It is expected that others will perceive differences, which while differing from the foregoing, do not depart from the spirit and scope of the disclosure herein described and claimed. All patents, patent publications, and other references cited herein are incorporated herein by reference in their entirety.

1. A composition comprising N-acetylneuraminic acid methyl ester, wherein the pH of the composition is between 3.0-6.8.

2. The composition of claim 1, wherein the pH of the composition is between 3.5-6.0, between 4.0-5.5, or between 4.5-5.0.

3-4. (canceled)

5. The composition of claim 1, wherein the composition further comprises N-acetylneuraminic acid, and wherein the composition comprises N-acetylneuraminic acid methyl ester and N-acetylneuraminic acid at a ratio of between 1:1 and 5:1 or between 1.25:1 and 4:1 (N-acetylneuraminic acid methyl ester:N-acetylneuraminic acid).

6-7. (canceled)

8. The composition of claim 1, further comprising sodium citrate at a ratio of between 1:1 and 5:1 (N-acetylneuraminic acid methyl ester:citrate).

9-10. (canceled)

11. The composition of claim 1, further comprising sodium acetate at a ratio of between 1:1 and 5:1 (N-acetylneuraminic acid methyl ester:acetate).

12. (canceled)

13. The composition of claim 1, further comprising glucose.

14. (canceled)

15. The composition of claim 1, further comprising a pharmaceutically acceptable carrier.

16. A composition comprising an analog or a derivative of N-acetylneuraminic acid,
wherein the analog or derivative of N-acetylneuraminic acid comprises the general chemical structure of 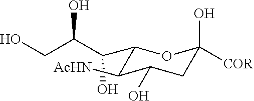
wherein R is a hydroxyl, hydrogen, alkoxy, alkyl, cycloalkyl, sodium (Na), substituted alkyl, substituted cycloalkyl, aryl, or substituted aryl, ether, HN, H₂N, NHAc, thioester, S—CH₂—CH₃, disulfide ester, S—CH₃, disulfide methyl, methionine, methionine-zinc or phenol or phenol derivatives;
wherein when dissolved the pH of the compositions or the products comprising at least one of the analogs or derivatives of N-acetylneuraminic acid is between 3.0-6.8, preferably 3.5-6.0, preferably 4.0-5.5, most preferably 4.5-5.0.

17. The composition of claim 16, further comprising N-acetylneuraminic acid, wherein the N-acetylneuraminic acid is present at a ratio of between 1:1 and 5:1 or between 1.25:1 and 4:1 (analog of N-acetylneuraminic acid:N-acetylneuraminic acid).

18-21. (canceled)

22. A method for preventing and/or treating a saccharide related disease, comprising administering to a subject an effective amount of the composition of claim 1.

23. The method of claim 22, wherein the saccharide related disease comprises an infectious disease, infection, infection-related disease, complication or sequela of an infection, adverse reaction to vaccine or therapeutic antibody, cytokine storm or cytokine release syndrome (CRS), inflammation, inflammatory respiratory disease, inflammatory gastrointestinal disease, diarrhea, inflammatory bowel disease (IBD), dehydration, arthritis, autoimmune disease, allergy, or cancer.

24. The method of claim 23, wherein the infectious disease or infection is from a respiratory virus, influenza virus, respiratory enterovirus, adenovirus, coronavirus, rhinovirus, respiratory syncytial virus, or B virus.

25. (canceled)

26. The method of claim 24, wherein the influenza virus is a type A, B, or C influenza virus.

27. The method of claim 26, wherein the type A influenza virus is H1N1, H3N2, H5N1, H7N9, H7N8, H9N2, or a variant thereof.

28. The method of claim 24, wherein the virus is a coronavirus.

29. The method of claim 28, wherein the coronavirus is a severe acute respiratory syndrome (SARS) virus, SARS-CoV-2 virus, Middle East respiratory syndrome (MERS) virus, or avian infectious bronchitis virus (IBV).

30. The method of claim 22, wherein the subject is or is identified as being a COVID-19 long hauler.

31. The method of claim 24, wherein the enterovirus is a rotavirus, reovirus, Coxsackie virus, Echoviruses, Enteroviruses, Polioviruses, norovirus, coronavirus, Norwalk virus, cytomegalovirus (CMV), herpes simplex virus, hepatitis virus, enterocytopathic human orphan (ECHO) virus, porcine enterovirus (PEV), porcine enterovirus (PTV), foot and mouth disease (HFMD), human enterovirus 71, or porcine epidemic diarrhea virus.

32. The method of claim 23, wherein the adverse reaction is to vaccine against a virus, and wherein the virus is a respiratory virus, influenza virus, respiratory enterovirus, adenovirus, coronavirus, rhinovirus, respiratory syncytial virus, or B virus.

33. (canceled)

34. The method of claim 23, wherein the complication or sequela is from a viral infection, wherein the virus is a respiratory virus, influenza virus, respiratory enterovirus, adenovirus, coronavirus, rhinovirus, respiratory syncytial virus, or B virus, and wherein the complication or sequela comprises inflammation and/or damage to a lung, kidney, cardiovascular system or organ, nervous system or organ, liver, or digestive system or organ.

35-36. (canceled)

37. The method of claim 23, wherein the complication or sequela comprises acute respiratory distress syndrome (ARDS) or acute respiratory diseases (ARD).

38. The method of claim 23, wherein the infection-related disease comprises abortion, postpartum labor, still birth, neonatal death, or neonatal sudden death.

39. The method of claim 22, wherein the method comprises administering to the subject N-acetylneuraminic acid methyl ester or an analog of N-acetylneuraminic acid at a dose of about 0.01 mg/kg to about 20 mg/kg& wherein the administration is given subcutaneously, topically, orally, intramuscularly, intravenously, intraperitoneally, intracavitally, transdermally, or via inhalation.

40. (canceled)

41. The method of claim 22, wherein the subject is a human.

42. The method of claim 41, wherein the human is a newborn, 1-12 months old, 18 years of age or younger, 18 years of age or older, pregnant, a breastfeeding infant, or a lactating mother.

43-48. (canceled)

49. The method of claim 22, wherein the subject is a livestock animal, cow, pig, horse, sheep, goat, llama, donkey, chicken, duck, goose, turkey, pigeon, dog, cat, rodent, bird, fish, shrimp, oyster, crustacean, or mollusk.
